SEQUENCE LISTING (146 sequences)

SEQ ID NO: 1 - protein, 8 aa, synthetic construct
  REGION 1..8: note = CDR-H1
GFSFSSHP

SEQ ID NO: 2 - protein, 8 aa, synthetic construct
  REGION 1..8: note = CDR-H2
ISGAGSHT

SEQ ID NO: 3 - protein, 21 aa, synthetic construct
  REGION 1..21: note = CDR-H3
AKGASSGSDSDRWTYDGGIDE

SEQ ID NO: 4-5 - skipped (2 entries)

SEQ ID NO: 6 - protein, 10 aa, synthetic construct
  REGION 1..10: note = CDR-L3
GNADSSGTII

SEQ ID NO: 7 - protein, 127 aa, synthetic construct
  REGION 1..127: note = VH
AVTLDESGGGLQTPGGALSLVCKASGFSFSSHPMMWVRQAPGKGLEFVATISGAGSHTYYGSAVKGRTTISRDNGQSTVRLQLNNLRAEDTATYYCAKGASSGSDSDRWTYDGGIDEWGHGTEVIVS

SEQ ID NO: 8 - protein, 102 aa, synthetic construct
  REGION 1..102: note = VL
ALTQPSSVSANPGETIKITCSGGSYYYGWYQQKSPGSAPVTVIYYNDNRPSNIPSRFSGSASGSTHTLTITGVQADDEAVYYCGNADSSGTIIFGAGTTLTV

SEQ ID NO: 9 - protein, 8 aa, synthetic construct
  REGION 1..8: note = CDR-H1
GIDLSSNA

SEQ ID NO: 10 - protein, 7 aa, synthetic construct
  REGION 1..7: note = CDR-H2
MSISGNP

SEQ ID NO: 11 - protein, 15 aa, synthetic construct
  REGION 1..15: note = CDR-H3
AKSIHCTWWYCANVL

SEQ ID NO: 12 - protein, 6 aa, synthetic construct
  REGION 1..6: note = CDR-L1
QSISSY

SEQ ID NO: 13 - skipped

SEQ ID NO: 14 - protein, 14 aa, synthetic construct
  REGION 1..14: note = CDR-L3
QGYDSISGVNYYNF

SEQ ID NO: 15 - protein, 117 aa, synthetic construct
  REGION 1..117: note = VH
QSVEESGGRLVTPGTPLTLTCTVSGIDLSSNAISWVRQAPGKGLEYIGTMSISGNPNYASWAKGRFTISRTSTTVDLKMTSPTTEDTATYFCAKSIHCTWWYCANVLWGQGTLVTVS

SEQ ID NO: 16 - protein, 111 aa, synthetic construct
  REGION 1..111: note = VL
ALVMTQTPSSVEAAVGGTVTIKCQASQSISSYLAWYQQKLGQPPKLLIYSASILASGVSSRFKGSGSGTEFTLTISDLECADAATYYCQGYDSISGVNYYNFFGGGTKVVV

SEQ ID NO: 17 - protein, 8 aa, synthetic construct
  REGION 1..8: note = CDR-H1
GFDFSSHG

SEQ ID NO: 18 - protein, 8 aa, synthetic construct
  REGION 1..8: note = CDR-H2
ISNTGSFT

SEQ ID NO: 19 - protein, 15 aa, synthetic construct
  REGION 1..15: note = CDR-H3
ARSSSAGGAADSIDA

SEQ ID NO: 20 - protein, 8 aa, synthetic construct
  REGION 1..8: note = CDR-L1
GNSYGIYY

SEQ ID NO: 21 - skipped

SEQ ID NO: 22 - protein, 10 aa, synthetic construct
  REGION 1..10: note = CDR-L3
GSLDNSSAGI

SEQ ID NO: 23 - protein, 121 aa, synthetic construct
  REGION 1..121: note = VH
AVTLDESGGGLQTPGGALSLVCKASGFDFSSHGMFWVRQAPGKGLEYVAEISNTGSFTAYGAAVKGRATISRDDGQSTVRLQLNNLRAEDTGTYYCARSSSAGGAADSIDAWGHGTEVIVS

SEQ ID NO: 24 - protein, 107 aa, synthetic construct
  REGION 1..107: note = VL
ALTQPSSVSANPGETVKITCSGGGNSYGIYYYGWCQQKSPGSAPVTVIYSNDKRPSNIPSRFSGSTSGSTSTLTITGVQVEDEAVYYCGSLDNSSAGIFGAGTTLTV

SEQ ID NO: 25 - protein, 8 aa, synthetic construct
  REGION 1..8: note = CDR-H1
GFTFNNYD

SEQ ID NO: 26 - protein, 8 aa, synthetic construct
  REGION 1..8: note = CDR-H2
IGNDGSWT

SEQ ID NO: 27 - protein, 19 aa, synthetic construct
  REGION 1..19: note = CDR-H3
AKSAGGGGLISYTADYIDA

SEQ ID NO: 28-29 - skipped (2 entries)

SEQ ID NO: 30 - protein, 11 aa, synthetic construct
  REGION 1..11: note = CDR-L3
GSEDKTTGYAA

SEQ ID NO: 31 - protein, 125 aa, synthetic construct
  REGION 1..125: note = VH
AVTLDESGGGLQTPGGALSLVCKASGFTFNNYDMGWVRQAPGKGLEFVAGIGNDGSWTGYGSAVKGRATISRDNGQSTVRLQLNNLRAEDTGTYFCAKSAGGGGLISYTADYIDAWGHGTEVIVS

SEQ ID NO: 32 - protein, 102 aa, synthetic construct
  REGION 1..102: note = VL
ALTQPSSVSANLGGTVKITCSGGGNYGWYQQKSPGSAPVTLIYQNTNRPSNIPSRFSGSKSGSTATLTITGVQADDEAVYYCGSEDKTTGYAAFGAGTTLTV

SEQ ID NO: 33 - protein, 9 aa, synthetic construct
  REGION 1..9: note = CDR-H1
GFDFSNYYY

SEQ ID NO: 34 - protein, 9 aa, synthetic construct
  REGION 1..9: note = CDR-H2
IAAGSGGYT

SEQ ID NO: 35 - protein, 11 aa, synthetic construct
  REGION 1..11: note = CDR-H3
ASSGDYKGFNL

SEQ ID NO: 36 - protein, 6 aa, synthetic construct
  REGION 1..6: note = CDR-L1
QSISSY

SEQ ID NO: 37 - skipped

SEQ ID NO: 38 - protein, 12 aa, synthetic construct
  REGION 1..12: note = CDR-L3
QGYYYSFSDIFA

SEQ ID NO: 39 - protein, 117 aa, synthetic construct
  REGION 1..117: note = VH
QSVEESGGGLVKPGGTLTLTCTASGFDFSNYYYMCWVRQAPGKGLEWIACIAAGSGGYTYYASWAKGRFTISNTSSTTVTLQMTSLTAADTATWFCASSGDYKGFNLWGPGTLVTVS

SEQ ID NO: 40 - protein, 109 aa, synthetic construct
  REGION 1..109: note = VL
ALVMTQTPASVEAAVGGTVTINCQASQSISSYLAWYQQKRGQPPKLLIYGASNLASGVPSRFKGSGSGTEYTLTISGVQCADAATYYCQGYYYSFSDIFAFGGGTEVVV

SEQ ID NO: 41 - protein, 8 aa, synthetic construct
  REGION 1..8: note = CDR-H1
GFTFSDYS

SEQ ID NO: 42 - protein, 8 aa, synthetic construct
  REGION 1..8: note = CDR-H2
IHCTGSYT

SEQ ID NO: 43 - protein, 23 aa, synthetic construct
  REGION 1..23: note = CDR-H3
AKSAASGSCTSRSYIYTADTIDA

SEQ ID NO: 44-45 - skipped (2 entries)

SEQ ID NO: 46 - protein, 11 aa, synthetic construct
  REGION 1..11: note = CDR-L3
GAYDGNTDAGI

SEQ ID NO: 47 - protein, 129 aa, synthetic construct
  REGION 1..129: note = VH
AVTLDESGGGLQTPGGALSLVCKASGFTFSDYSMQWVRQAPGKGLEWVAGIHCTGSYTYYGPAVKGRVTISRDNGQSTVRLQLNNLRAEDTGTYYCAKSAASGSCTSRSYIYTADTIDAWGHGTEVIVS

SEQ ID NO: 48 - protein, 103 aa, synthetic construct
  REGION 1..103: note = VL
ALTQPSSVSANPGETVKITCSGSSSYYGWYQQKSPGSVPVTLIYYNNKRPSDIPSRFSGSTSGSTNTLTITGVQAEDEAVYFCGAYDGNTDAGIFGAGTTLTV

SEQ ID NO: 49 - protein, 8 aa, synthetic construct
  REGION 1..8: note = CDR-H1
GFTFSSYD

SEQ ID NO: 50 - protein, 8 aa, synthetic construct
  REGION 1..8: note = CDR-H2
INADGSNT

SEQ ID NO: 51 - protein, 21 aa, synthetic construct
  REGION 1..21: note = CDR-H3
ARSFGTGGYYHIAIGPGFIDA

SEQ ID NO: 52 - protein, 4 aa, synthetic construct
  REGION 1..4: note = CDR-L1
SNSG

SEQ ID NO: 53 - skipped

SEQ ID NO: 54 - protein, 12 aa, synthetic construct
  REGION 1..12: note = CDR-L3
GNEDSSDPTGAI

SEQ ID NO: 55 - protein, 127 aa, synthetic construct
  REGION 1..127: note = VH
AVTLDESGGGLQTPGGALSLICKGSGFTFSSYDMGWMRQAPGKGLEFVAGINADGSNTGYGPAVKGRATISRDNGQSTVRLQLNNLRAEDTATYYCARSFGTGGYYHIAIGPGFIDAWGHGTEVIVS

SEQ ID NO: 56 - protein, 105 aa, synthetic construct
  REGION 1..105: note = VL
ALTQPSSVSANPGETVKITCSGDSNSGYGWYQQKSPGSALVTVIYWNDKRPSNIPSRFSGSTSGSTNTLTITGVQADDEAVYFCGNEDSSDPTGAIFGAGTTLTV

SEQ ID NO: 57 - protein, 8 aa, synthetic construct
  REGION 1..8: note = CDR-H1
GFTFSDYG

SEQ ID NO: 58 - protein, 10 aa, synthetic construct
  REGION 1..10: note = CDR-H2
ISSSADGSST

SEQ ID NO: 59 - protein, 17 aa, synthetic construct
  REGION 1..17: note = CDR-H3
AKAAYGSAWNYAIIIDA

SEQ ID NO: 60-61 - skipped (2 entries)

SEQ ID NO: 62 - protein, 11 aa, synthetic construct
  REGION 1..11: note = CDR-L3
GGYDSSTYAGI

SEQ ID NO: 63 - protein, 125 aa, synthetic construct
  REGION 1..125: note = VH
AVTLDESGGGLQTPGGALSLVCKASGFTFSDYGMGWTRQAPGKGLEYVASISSSADGSSTWYAPAVKGRATISRDNGQSTVRLQLNNLRAEDTATYYCAKAAYGSAWNYAIIIDAWGHGTEVIVS

SEQ ID NO: 64 - protein, 103 aa, synthetic construct
  REGION 1..103: note = VL
ALTQPSSVSANLGGTVEITCSGGSDNYGWFQQKSPGSAPVTVIYNNNNRPSDIPSRFSGSKSGSTGTLTITGVQAEDEAVYFCGGYDSSTYAGIFGAGTTLTV

SEQ ID NO: 65 - protein, 8 aa, synthetic construct
  REGION 1..8: note = CDR-H1
GFTFSNYA

SEQ ID NO: 66 - protein, 8 aa, synthetic construct
  REGION 1..8: note = CDR-H2
IRGSGGTT

SEQ ID NO: 67 - protein, 14 aa, synthetic construct
  REGION 1..14: note = CDR-H3
AKNKAYYDSSGYDY

SEQ ID NO: 68 - protein, 9 aa, synthetic construct
  REGION 1..9: note = CDR-L1
SSNIGVGYD

SEQ ID NO: 69 - skipped

SEQ ID NO: 70 - protein, 11 aa, synthetic construct
  REGION 1..11: note = CDR-L3
QSYDSSLSGVV

SEQ ID NO: 71 - protein, 120 aa, synthetic construct
  REGION 1..120: note = VH
QVQLVQSGGGLVQPGGSLRLSCAASGFTFSNYAMTWVRQAPGKGLEWVSAIRGSGGTTYYADSVKGRSTISRDNSKNTLYLQMNSLSAEDTAVYYCAKNKAYYDSSGYDYWGQGTTVTVS

SEQ ID NO: 72 - protein, 110 aa, synthetic construct
  REGION 1..110: note = VL
QSVLTQPPSVSGAPGQRVTISCTGSSSNIGVGYDVHWYQQLPGTAPKLLIYGNSNRPSGVPDRFSGSKSGTSASLAITGLQAEDEADYYCQSYDSSLSGVVFGGGTKLTV

SEQ ID NO: 73 - protein, 8 aa, synthetic construct
  REGION 1..8: note = CDR-H1
GYTFTSYG

SEQ ID NO: 74 - protein, 8 aa, synthetic construct
  REGION 1..8: note = CDR-H2
ISAYNGNT

SEQ ID NO: 75 - protein, 11 aa, synthetic construct
  REGION 1..11: note = CDR-H3
ARDGNWRGFDY

SEQ ID NO: 76 - protein, 8 aa, synthetic construct
  REGION 1..8: note = CDR-L1
SSNIGSNY

SEQ ID NO: 77 - skipped

SEQ ID NO: 78 - protein, 12 aa, synthetic construct
  REGION 1..12: note = CDR-L3
AAWDDSLSGSWV

SEQ ID NO: 79 - protein, 117 aa, synthetic construct
  REGION 1..117: note = VH
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARDGNWRGFDYWGQGTLVTVS

SEQ ID NO: 80 - protein, 110 aa, synthetic construct
  REGION 1..110: note = VL
QSVLTQPPSASGTPGQRVTISCSGSSSNIGSNYVYWYQQLPGTAPKLLIYRNNQRPSGVPDRFSGSKSGASASLAISGLRSEDEADYYCAAWDDSLSGSWVFGGGTKVTV

SEQ ID NO: 81 - protein, 8 aa, synthetic construct
  REGION 1..8: note = CDR-H1
GFTFSSYG

SEQ ID NO: 82 - protein, 8 aa, synthetic construct
  REGION 1..8: note = CDR-H2
ISYDGSNK

SEQ ID NO: 83 - protein, 16 aa, synthetic construct
  REGION 1..16: note = CDR-H3
ANYYDSSGYLPEYFQH

SEQ ID NO: 84 - protein, 9 aa, synthetic construct
  REGION 1..9: note = CDR-L1
SSDVGGYNY

SEQ ID NO: 85 - skipped

SEQ ID NO: 86 - protein, 11 aa, synthetic construct
  REGION 1..11: note = CDR-L3
SSYTSSSTYWV

SEQ ID NO: 87 - protein, 122 aa, synthetic construct
  REGION 1..122: note = VH
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCANYYDSSGYLPEYFQHWGQGTLVTVS

SEQ ID NO: 88 - protein, 110 aa, synthetic construct
  REGION 1..110: note = VL
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTYWVFGGGTKVTV

SEQ ID NO: 89 - protein, 8 aa, synthetic construct
  REGION 1..8: note = CDR-H1
GFTFSSYG

SEQ ID NO: 90 - protein, 8 aa, synthetic construct
  REGION 1..8: note = CDR-H2
IWYDGSNK

SEQ ID NO: 91 - protein, 14 aa, synthetic construct
  REGION 1..14: note = CDR-H3
ATQHQVTDGEPFDY

SEQ ID NO: 92 - protein, 8 aa, synthetic construct
  REGION 1..8: note = CDR-L1
SSNIGSNY

SEQ ID NO: 93 - skipped

SEQ ID NO: 94 - protein, 11 aa, synthetic construct
  REGION 1..11: note = CDR-L3
AAWDDSLSGPV

SEQ ID NO: 95 - protein, 120 aa, synthetic construct
  REGION 1..120: note = VH
EVQLVQSGGGVVQPGRSLRLSCAASGFTFSSYGMHWVRQAPGKGLEWVAVIWYDGSNKYYADSVKGRFTISRDNSKNTLYLQMNSLRAEDTAVYYCATQHQVTDGEPFDYWGQGTLVTVS

SEQ ID NO: 96 - protein, 109 aa, synthetic construct
  REGION 1..109: note = VL
QSVLTQPPSASGTPGQRVTISCSGSSSNIGSNYVYWYQQLPGTAPKLLIYRNNQRPSGVPDRFSGSKSGTSASLAISGLRSEDEADYYCAAWDDSLSGPVFGGGTKLTV

SEQ ID NO: 97 - protein, 8 aa, synthetic construct
  REGION 1..8: note = CDR-H1
GFTFSSYS

SEQ ID NO: 98 - protein, 8 aa, synthetic construct
  REGION 1..8: note = CDR-H2
ISSSSSYI

SEQ ID NO: 99 - protein, 12 aa, synthetic construct
  REGION 1..12: note = CDR-H3
ANWSYGYRAFDY

SEQ ID NO: 100 - protein, 9 aa, synthetic construct
  REGION 1..9: note = CDR-L1
SSDVGGYNY

SEQ ID NO: 101 - skipped

SEQ ID NO: 102 - protein, 11 aa, synthetic construct
  REGION 1..11: note = CDR-L3
SSYTSSSTYVV

SEQ ID NO: 103 - protein, 118 aa, synthetic construct
  REGION 1..118: note = VH
EVQLVQSGGGLVKPGGSLRLSCAASGFTFSSYSMNWVRQAPGKGLEWVSSISSSSSYIYYADSVKGRFTISRDNAKNSLYLQMNSLRAEDTAVYYCANWSYGYRAFDYWGQGTLVTVS

SEQ ID NO: 104 - protein, 110 aa, synthetic construct
  REGION 1..110: note = VL
QSAPTQPASVSGSPGQSITISCTGTSSDVGGYNYVSWYQQHPGKAPKLMIYDVSNRPSGVSNRFSGSKSGNTASLTISGLQAEDEADYYCSSYTSSSTYVVFGGGTKVTV

SEQ ID NO: 105 - protein, 8 aa, synthetic construct
  REGION 1..8: note = CDR-H1
GFTFSSYS

SEQ ID NO: 106 - protein, 8 aa, synthetic construct
  REGION 1..8: note = CDR-H2
ISSSSSYI

SEQ ID NO: 107 - protein, 12 aa, synthetic construct
  REGION 1..12: note = CDR-H3
ASSIAVAGIMGY

SEQ ID NO: 108 - protein, 6 aa, synthetic construct
  REGION 1..6: note = CDR-L1
QSVSSN

SEQ ID NO: 109 - skipped

SEQ ID NO: 110 - protein, 8 aa, synthetic construct
  REGION 1..8: note = CDR-L3
QQYNNWPT

SEQ ID NO: 111 - protein, 118 aa, synthetic construct
  REGION 1..118: note = VH
QVQLVQSGGGLVKPGGSLRLSCAASGFTFSSYSMNWVRQAPGKGLEWVSSISSSSSYIYYADSVKGRFTISRDNAKNSLYLQMNSLRAEDTAVYYCASSIAVAGIMGYWGQGTLVTVS

SEQ ID NO: 112 - protein, 105 aa, synthetic construct
  REGION 1..105: note = VL
EIVLTQSPATLSVSPGERATLSCRASQSVSSNLAWYQQKPGQAPRLLIYGASTRATGIPARFSGSGSGTEFTLTISSLQSEDFAVYYCQQYNNWPTFGQGTKLEI

SEQ ID NO: 113 - protein, 8 aa, synthetic construct
  REGION 1..8: note = CDR-H1
GGTFSSYT

SEQ ID NO: 114 - protein, 8 aa, synthetic construct
  REGION 1..8: note = CDR-H2
IIPILGIA

SEQ ID NO: 115 - protein, 12 aa, synthetic construct
  REGION 1..12: note = CDR-H3
ATDVEMATRVDY

SEQ ID NO: 116 - protein, 8 aa, synthetic construct
  REGION 1..8: note = CDR-L1
SSNIGSNY

SEQ ID NO: 117 - skipped

SEQ ID NO: 118 - protein, 11 aa, synthetic construct
  REGION 1..11: note = CDR-L3
AAWDDSLSGAV

SEQ ID NO: 119 - protein, 118 aa, synthetic construct
  REGION 1..118: note = VH
QVQLVQSGAEVKKPGSSVKVSCKASGGTFSSYTISWVRQAPGQGLEWMGRIIPILGIANYAQKFQGRVTITADKSTSTAYMELSSLRSEDTAVYYCATDVEMATRVDYWGQGTLVTVS

SEQ ID NO: 120 - protein, 109 aa, synthetic construct
  REGION 1..109: note = VL
QSVLTQPPSASGTPGQRVTIPCSGSSSNIGSNYVYWYQQLPGTAPKLLIYRNNQRPSGVPDRFSGSKSGTSASLAISGLRSEDEADYYCAAWDDSLSGAVFGGGTKLTV

SEQ ID NO: 121 - protein, 8 aa, synthetic construct
  REGION 1..8: note = CDR-H1
GYSFTSYW

SEQ ID NO: 122 - protein, 8 aa, synthetic construct
  REGION 1..8: note = CDR-H2
IYPGDSDT

SEQ ID NO: 123 - protein, 11 aa, synthetic construct
  REGION 1..11: note = CDR-H3
ARGDYSSGWYY

SEQ ID NO: 124 - protein, 8 aa, synthetic construct
  REGION 1..8: note = CDR-L1
SPNVGTNY

SEQ ID NO: 125 - skipped

SEQ ID NO: 126 - protein, 11 aa, synthetic construct
  REGION 1..11: note = CDR-L3
ETWDNSLNAYV

SEQ ID NO: 127 - protein, 117 aa, synthetic construct
  REGION 1..117: note = VH
QVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARGDYSSGWYYWGQGTLVTVS

SEQ ID NO: 128 - protein, 109 aa, synthetic construct
  REGION 1..109: note = VL
QSVLTQPPSVSAAAGQKVTISCSGSSPNVGTNYVSWYQQLPGTAPKLLILGNDRRPSAIPDRFSGSKSGTSATLGITGLQTGDEADYYCETWDNSLNAYVFGTGTQLTV

SEQ ID NO: 129 - protein, 8 aa, synthetic construct
  REGION 1..8: note = CDR-H1
GYTFSTSG

SEQ ID NO: 130 - protein, 8 aa, synthetic construct
  REGION 1..8: note = CDR-H2
INGYGSHR

SEQ ID NO: 131 - protein, 11 aa, synthetic construct
  REGION 1..11: note = CDR-H3
ARDHDWNGFDN

SEQ ID NO: 132 - protein, 8 aa, synthetic construct
  REGION 1..8: note = CDR-L1
SSNIGSNY

SEQ ID NO: 133 - skipped

SEQ ID NO: 134 - protein, 12 aa, synthetic construct
  REGION 1..12: note = CDR-L3
AAWDDSLSGLWV

SEQ ID NO: 135 - protein, 117 aa, synthetic construct
  REGION 1..117: note = VH
QVQLVQSGAEVKEPGASVKVSCKASGYTFSTSGISWVRQAPGQGLEWMGWINGYGSHRLFPQNFQGRLTLTTDTSTSTAYMEVRSLRSDDTAIYYCARDHDWNGFDNWGQGTLVTVS

SEQ ID NO: 136 - protein, 110 aa, synthetic construct
  REGION 1..110: note = VL
SYVLTQPPSASGTPGQRVTISCSGSSSNIGSNYVYWYQQLPGTAPKLLIYRNNQRPSGVPDRFSGSKSGTSASLAISGLRSEDEADYYCAAWDDSLSGLWVFGGGTKLTV

SEQ ID NO: 137 - protein, 346 aa, Homo sapiens [sequence omitted]

SEQ ID NO: 138 - protein, 119 aa, synthetic construct
QVQLVESGGGLVKPGGSLRLSCAASGFDFSNYYYMCWIRQAPGKGLEWVSCIAAGSGGYTYYASWAKGRFTISRDNAKNSLYLQMNSLRAEDTAVYYCASSGDYKGFNLWGQGTLVTVS

SEQ ID NO: 139 - protein, 119 aa, synthetic construct
LVQLVESGGGLVKPGGSLRLSCAASGFDFSNYYYMCWIRQAPGKGLEWVSCIAAGSGGYTYYASWAKGRFTISRDNAKNSLYLQMNSLRAEDTAVWYCASSGDYKGFNLWGQGTLVTVS

SEQ ID NO: 140 - protein, 119 aa, synthetic construct
QVQLQESGGGLVQPGGSLRLSCAASGFDFSNYYYMCWVRQAPGKGLEWVLCIAAGSGGYTYYASWAKGRFTISRDNSKNTLYLQMNSLRAEDTAIYYCASSGDYKGFNLWGQGTLVTVS

SEQ ID NO: 141 - protein, 119 aa, synthetic construct
EVQLVESGGGLVQPGGSLRLSCAASGFDFSNYYYMCWVRQAPGKGLEWVSCIAAGSGGYTYYASWAKGRFTISRDNSKNTLYLQMNSLRAEDTAVYYCASSGDYKGFNLWGQGTMVTVS

SEQ ID NO: 142 - protein, 119 aa, synthetic construct
EVQLVESGGGLVQPGGSLRLSCAASGFDFSNYYYMCWVRQAPGKGLEWVSCIAAGSGGYTYYASWAKGRFTISRDNSKNTLYLQMNSLRAEDTAVYYCASSGDYKGFNLWGQGTLVTVS

SEQ ID NO: 143 - protein, 109 aa, synthetic construct
EIVMTQSPSSLSASVGDRVTITCQASQSISSYLAWYQQKPGKAPKLLIYGASNLASGVPSRFSGSGSGTDFTLTISSLQPEDFATYYCQGYYYSFSDIFAFGGGTKVEI

SEQ ID NO: 144 - protein, 109 aa, synthetic construct
DIVMTQSPSSLSASVGDRVTITCQASQSISSYLAWYQQKPGKPPKLLIYGASNLASGVPSRFSGSGSGTDFTLTISSLQPEDVATYYCQGYYYSFSDIFAFGGGTKVEI

SEQ ID NO: 145 - protein, 109 aa, synthetic construct
AIRIAQSPSSLSASTGDRVTITCQASQSISSYLAWYQQKPGKAPKLLIYGASNLASGVPSRFSGSGSGTDFTLTISCLQSEDFATYYCQGYYYSFSDIFAFGQGTKVEI

SEQ ID NO: 146 - protein, 109 aa, synthetic construct
EIVMTQSPSSLSASVGDRVTITCQASQSISSYLAWYQQKPGKPPKLLIYGASNLASGVPSRFSGSGSGTDFTLTISSLQPEDVATYYCQGYYYSFSDIFAFGGGTKLEI

(19) United States
(12) Patent Application Publication
(10) Pub. No.: US 2025/0074999 A1
(43) Pub. Date: Mar. 6, 2025

(54) ANTIBODIES AGAINST LYPD3

(71) Applicant: Pentixapharm AG, Würzburg (DE)

(72) Inventors: Johanna GELLERT, Berlin (DE); Antje DANIELCZYK, Berlin (DE); Patrik KEHLER, Berlin (DE); Anika JÄKEL, Berlin (DE); Sven BAHRKE, Berlin (DE); Timo LISCHKE, Berlin (DE); Christoph GOLETZ, Berlin (DE); Anke FLECHNER, Berlin (DE); Lars STÖCKL, Berlin (DE); Stephanie GURKA, Berlin (DE)

(21) Appl. No.: 18/844,148

(22) PCT Filed: Mar. 16, 2023
(86) PCT No.: PCT/EP2023/056832

§ 371 Date: Sep. 5, 2024

(30) Foreign Application Priority Data
Mar. 16, 2022 (EP) 22162550.2

(51) Int. Cl. C07K 16/30; A61K 47/68; A61P 35/00

(52) U.S. Cl. CPC C07K 16/30; A61K 47/68031; A61K 47/6851; A61P 35/00; C07K 2317/24; C07K 2317/52; C07K 2317/565; C07K 2317/77

(57) ABSTRACT

The present invention pertains to anti-LYPD3 antibodies which bind LYPD3 in an O-glycosylation-dependent manner. Thereby, the antibodies are specific for tumor-associated LYPD3. The invention further provides pharmaceutical compositions containing said anti-LYPD3 antibodies and their use in cancer treatment.

### FIELD OF THE INVENTION

The present invention pertains to the field of antibodies. In particular, anti-LYPD3 antibodies showing strong antigen binding in a glycosylation-dependent manner are provided. In specific embodiments, the present invention is directed to anti-LYPD3 antibodies for therapeutic and diagnostic use.

### BACKGROUND OF THE INVENTION

Today, antibodies are widely used agents in the field of medicine and research. In medicine, they find application in many different fields. For example, antibodies are used as therapeutic agents in the treatment and prophylaxis of a variety of diseases such as cancer, cardiovascular diseases, inflammatory diseases, macular degeneration, transplant rejection, multiple sclerosis, and viral infections. In these therapies, the antibody may possess therapeutic activity on its own, for example by blocking receptors or messenger molecules, thereby inhibiting their disease-relevant functions, or by recruiting and activating components of the patient's immune system.

In cancer treatment, a key characteristic of therapeutic antibodies is their specificity for tumor tissue. This means that the antibodies should target an epitope which is exclusively or predominantly found on cancer cells, but only to a low extent on cells of normal tissue. Thereby, the therapeutic activity of the antibody, e.g. inducing an immune response against the targeted cells or destroying the cells by a cytotoxic payload, specifically acts at the tumor site. Respective activity at normal tissue—due to binding of the antibody to non-tumor cells—may result in severe side effects. Increasing the specificity for tumor cells hence reduces the risk of adverse effects and enhances the safety of the envisioned therapy.

A possible cancer antigen is LYPD3 (C4.4A). This protein is a glycosylphosphatidylinositol (GPI)-anchored, highly glycosylated cell surface protein that has been shown to be upregulated in migrating keratinocytes during wound healing. It was first described as a metastasis-associated cell surface protein in rat pancreatic tumor cells and since then, has been associated with carcinogenesis in several different cancers. In cancer, LYPD3 has been suggested to be involved specifically in tumor cell invasion via interaction with the extracellular matrix.

LYPD3 is strongly overexpressed in non-small cell lung cancer (NSCLC) with preferential expression in squamous cell carcinoma (SCC) subtype compared with the other two most common NSCLC subtypes; adenocarcinoma (AC) and large-cell carcinoma (LCC). Lung cancer is the most frequently diagnosed cancer with an estimated 1.8 million new cases in 2012. NSCLC accounts for 85% of all lung cancers and SCC, being the second most frequent histologic subtype occurring in 30% of NSCLC cases, explains approximately 400,000 deaths per year worldwide.

Overexpression of LYPD3 has also been detected in SCC of the head and neck (HNSCC) including esophageal SCC (ESCC) subtype. On the transcriptional level, approximately 50% of primary lung cancers and 75% of lung cancer metastases express LYPD3 mRNA, whereas no expression has been detected in normal lung tissue. In addition, LYPD3 is expressed in colorectal and breast cancer.

Overexpression of LYPD3 has been shown to correlate with a malignant phenotype and poor prognosis in NSCLC, colorectal cancer and ESCC, while in breast cancer, it is associated with a good prognosis. Therefore, LYPD3 represents an interesting target for anti-tumor immunotherapy.

However, under normal physiologic conditions LYPD3 is expressed in skin keratinocytes, esophageal endothelial cells and placental cells. This may cause unwanted side effects in a cancer therapy with an antibody which cannot discriminate between cancer-associated LYPD3 and LYPD3 on normal tissue.

Therefore, there is a need in the art to provide tumor-specific anti-LYPD3 antibodies.

### SUMMARY OF THE INVENTION

The present inventors have developed anti-LYPD3 antibodies with enhanced tumor specificity. These antibodies bind to tumor-associated LYPD3 in an O-glycosylation-dependent manner, recognizing the O-glycan structures present on LYPD3 of cancer cells. O-glycosylation of cancer cells comprises high amounts of short chain structures, especially mono-, di- and trisaccharides such as the Thomsen-Friedenreich antigen (TF; Galβ1-3GalNAcα1-), sialylated Thomsen-Friedenreich antigen (sTF), the Thomsen nouvelle antigen (Tn; GalNAcα1-) and sialylated Thomsen nouvelle antigen (Siaα2-6GalNAcα1-). In contrast, in normal cells O-glycosylation encompasses much longer oligosaccharide chains. Since the developed antibodies specifically bind to LYPD3 carrying the short, cancer cell-derived O-glycosylation, they discriminate between tumor-associated LYPD3 (i.e. LYPD3 present on cancer cells) and LYPD3 on cells of normal tissue. Thereby, the antibodies according to the present invention preferentially bind to LYPD3 on tumor cells and hence, have a superior cancer specificity and reduced binding to normal tissue, resulting in lower risk for adverse effects in cancer immunotherapy.

Mature LYPD3 consists of two Ly-6 (leucocyte antigen 6)/uPAR/α-neurotoxin domains (LU domains) and is extensively modified by post-translational glycosylation, which include 5 N-glycosylation sites located in or close to the second LU domain and approximately 15 O-linked carbohydrates clustered in a Ser/Thr/Pro-rich region at the C terminus. The antibodies according to the present invention recognize LYPD3 in an O-glycosylation-dependent manner by binding to the C terminal STP-rich region at amino acids 234 to 303 of the human LYPD3 sequence (SEQ ID NO: 137), in particular at amino acids 247 to 297. In this region, the O-glycosylation sites of LYPD3 are located.

In view the above, in a first aspect, the present invention is directed to an antibody which is capable of specifically binding to glycosylated human LYPD3 at an epitope comprising an oligosaccharide structure selected from the group consisting of GalNAcα1-, sialylated GalNAcα1-, Galβ1-3GalNAcα1-, and sialylated Galβ1-3GalNAcα1-, which is attached to a serine or threonine residue of LYPD3.

In a second aspect, the present invention is directed to an anti-LYPD3 antibody which comprises
(i) a heavy chain variable region comprising the complementarity-determining regions (CDRs) CDR-H1 having the amino acid sequence of SEQ ID NO: 1, CDR-H2 having the amino acid sequence of SEQ ID NO: 2 and CDR-H3 having the amino acid sequence of SEQ ID NO: 3, and
   a light chain variable region comprising the complementarity-determining regions (CDRs) CDR-L1 having the amino acid sequence of SEQ ID NO: 4, CDR-L2 having the amino acid sequence of SEQ ID NO: 5 and CDR-L3 having the amino acid sequence of SEQ ID NO: 6; or
(ii) a heavy chain variable region comprising the complementarity-determining regions (CDRs) CDR-H1 having the amino acid sequence of SEQ ID NO: 9, CDR-H2 having the amino acid sequence of SEQ ID NO: 10 and CDR-H3 having the amino acid sequence of SEQ ID NO: 11, and
   a light chain variable region comprising the complementarity-determining regions (CDRs) CDR-L1 having the amino acid sequence of SEQ ID NO: 12, CDR-L2 having the amino acid sequence of SEQ ID NO: 13 and CDR-L3 having the amino acid sequence of SEQ ID NO: 14; or
(iii) a heavy chain variable region comprising the complementarity-determining regions (CDRs) CDR-H1 having the amino acid sequence of SEQ ID NO: 17, CDR-H2 having the amino acid sequence of SEQ ID NO: 18 and CDR-H3 having the amino acid sequence of SEQ ID NO: 19, and
   a light chain variable region comprising the complementarity-determining regions (CDRs) CDR-L1 having the amino acid sequence of SEQ ID NO: 20, CDR-L2 having the amino acid sequence of SEQ ID NO: 21 and CDR-L3 having the amino acid sequence of SEQ ID NO: 22; or
(iv) a heavy chain variable region comprising the complementarity-determining regions (CDRs) CDR-H1 having the amino acid sequence of SEQ ID NO: 25, CDR-H2 having the amino acid sequence of SEQ ID NO: 26 and CDR-H3 having the amino acid sequence of SEQ ID NO: 27, and
   a light chain variable region comprising the complementarity-determining regions (CDRs) CDR-L1 having the amino acid sequence of SEQ ID NO: 28, CDR-L2 having the amino acid sequence of SEQ ID NO: 29 and CDR-L3 having the amino acid sequence of SEQ ID NO: 30; or
(v) a heavy chain variable region comprising the complementarity-determining regions (CDRs) CDR-H1 having the amino acid sequence of SEQ ID NO: 33, CDR-H2 having the amino acid sequence of SEQ ID NO: 34 and CDR-H3 having the amino acid sequence of SEQ ID NO: 35, and
   a light chain variable region comprising the complementarity-determining regions (CDRs) CDR-L1 having the amino acid sequence of SEQ ID NO: 36, CDR-L2 having the amino acid sequence of SEQ ID NO: 37 and CDR-L3 having the amino acid sequence of SEQ ID NO: 38; or
(vi) a heavy chain variable region comprising the complementarity-determining regions (CDRs) CDR-H1 having the amino acid sequence of SEQ ID NO: 41, CDR-H2 having the amino acid sequence of SEQ ID NO: 42 and CDR-H3 having the amino acid sequence of SEQ ID NO: 43, and
   a light chain variable region comprising the complementarity-determining regions (CDRs) CDR-L1 having the amino acid sequence of SEQ ID NO: 44, CDR-L2 having the amino acid sequence of SEQ ID NO: 45 and CDR-L3 having the amino acid sequence of SEQ ID NO: 46; or
(vii) a heavy chain variable region comprising the complementarity-determining regions (CDRs) CDR-H1 having the amino acid sequence of SEQ ID NO: 49, CDR-H2 having the amino acid sequence of SEQ ID NO: 50 and CDR-H3 having the amino acid sequence of SEQ ID NO: 51, and
   a light chain variable region comprising the complementarity-determining regions (CDRs) CDR-L1 having the amino acid sequence of SEQ ID NO: 52, CDR-L2 having the amino acid sequence of SEQ ID NO: 53 and CDR-L3 having the amino acid sequence of SEQ ID NO: 54; or
(viii) a heavy chain variable region comprising the complementarity-determining regions (CDRs) CDR-H1 having the amino acid sequence of SEQ ID NO: 57, CDR-H2 having the amino acid sequence of SEQ ID NO: 58 and CDR-H3 having the amino acid sequence of SEQ ID NO: 59, and
   a light chain variable region comprising the complementarity-determining regions (CDRs) CDR-L1 having the amino acid sequence of SEQ ID NO: 60, CDR-L2 having the amino acid sequence of SEQ ID NO: 61 and CDR-L3 having the amino acid sequence of SEQ ID NO: 62; or
(ix) a heavy chain variable region comprising the complementarity-determining regions (CDRs) CDR-H1 having the amino acid sequence of SEQ ID NO: 65, CDR-H2 having the amino acid sequence of SEQ ID NO: 66 and CDR-H3 having the amino acid sequence of SEQ ID NO: 67, and
   a light chain variable region comprising the complementarity-determining regions (CDRs) CDR-L1 having the amino acid sequence of SEQ ID NO: 68, CDR-L2 having the amino acid sequence of SEQ ID NO: 69 and CDR-L3 having the amino acid sequence of SEQ ID NO: 70; or
(x) a heavy chain variable region comprising the complementarity-determining regions (CDRs) CDR-H1 having the amino acid sequence of SEQ ID NO: 73, CDR-H2 having the amino acid sequence of SEQ ID NO: 74 and CDR-H3 having the amino acid sequence of SEQ ID NO: 75, and
   a light chain variable region comprising the complementarity-determining regions (CDRs) CDR-L1 having the amino acid sequence of SEQ ID NO: 76, CDR-L2 having the amino acid sequence of SEQ ID NO: 77 and CDR-L3 having the amino acid sequence of SEQ ID NO: 78; or
(xi) a heavy chain variable region comprising the complementarity-determining regions (CDRs) CDR-H1 having the amino acid sequence of SEQ ID NO: 81, CDR-H2 having the amino acid sequence of SEQ ID NO: 82 and CDR-H3 having the amino acid sequence of SEQ ID NO: 83, and
   a light chain variable region comprising the complementarity-determining regions (CDRs) CDR-L1 having the amino acid sequence of SEQ ID NO: 84, CDR-L2 having the amino acid sequence of SEQ ID NO: 85 and CDR-L3 having the amino acid sequence of SEQ ID NO: 86; or
(xii) a heavy chain variable region comprising the complementarity-determining regions (CDRs) CDR-H1 having the amino acid sequence of SEQ ID NO: 89, CDR-H2 having the amino acid sequence of SEQ ID NO: 90 and CDR-H3 having the amino acid sequence of SEQ ID NO: 91, and
   a light chain variable region comprising the complementarity-determining regions (CDRs) CDR-L1 having the amino acid sequence of SEQ ID NO: 92, CDR-L2 having the amino acid sequence of SEQ ID NO: 93 and CDR-L3 having the amino acid sequence of SEQ ID NO: 94; or
(xiii) a heavy chain variable region comprising the complementarity-determining regions (CDRs) CDR-H1 having the amino acid sequence of SEQ ID NO: 97, CDR-H2 having the amino acid sequence of SEQ ID NO: 98 and CDR-H3 having the amino acid sequence of SEQ ID NO: 99, and
   a light chain variable region comprising the complementarity-determining regions (CDRs) CDR-L1 having the amino acid sequence of SEQ ID NO: 100, CDR-L2 having the amino acid sequence of SEQ ID NO: 101 and CDR-L3 having the amino acid sequence of SEQ ID NO: 102; or
(xiv) a heavy chain variable region comprising the complementarity-determining regions (CDRs) CDR-H1 having the amino acid sequence of SEQ ID NO: 105, CDR-H2 having the amino acid sequence of SEQ ID NO: 106 and CDR-H3 having the amino acid sequence of SEQ ID NO: 107, and
   a light chain variable region comprising the complementarity-determining regions (CDRs) CDR-L1 having the amino acid sequence of SEQ ID NO: 108, CDR-L2 having the amino acid sequence of SEQ ID NO: 109 and CDR-L3 having the amino acid sequence of SEQ ID NO: 110; or
(xv) a heavy chain variable region comprising the complementarity-determining regions (CDRs) CDR-H1 having the amino acid sequence of SEQ ID NO: 113, CDR-H2 having the amino acid sequence of SEQ ID NO: 114 and CDR-H3 having the amino acid sequence of SEQ ID NO: 115, and
   a light chain variable region comprising the complementarity-determining regions (CDRs) CDR-L1 having the amino acid sequence of SEQ ID NO: 116, CDR-L2 having the amino acid sequence of SEQ ID NO: 117 and CDR-L3 having the amino acid sequence of SEQ ID NO: 118; or
(xvi) a heavy chain variable region comprising the complementarity-determining regions (CDRs) CDR-H1 having the amino acid sequence of SEQ ID NO: 121, CDR-H2 having the amino acid sequence of SEQ ID NO: 122 and CDR-H3 having the amino acid sequence of SEQ ID NO: 123, and
   a light chain variable region comprising the complementarity-determining regions (CDRs) CDR-L1 having the amino acid sequence of SEQ ID NO: 124, CDR-L2 having the amino acid sequence of SEQ ID NO: 125 and CDR-L3 having the amino acid sequence of SEQ ID NO: 126; or
(xvii) a heavy chain variable region comprising the complementarity-determining regions (CDRs) CDR-H1 having the amino acid sequence of SEQ ID NO: 129, CDR-H2 having the amino acid sequence of SEQ ID NO: 130 and CDR-H3 having the amino acid sequence of SEQ ID NO: 131, and
   a light chain variable region comprising the complementarity-determining regions (CDRs) CDR-L1 having the amino acid sequence of SEQ ID NO: 132, CDR-L2 having the amino acid sequence of SEQ ID NO: 133 and CDR-L3 having the amino acid sequence of SEQ ID NO: 134.

In a third aspect, the present invention provides a nucleic acid encoding the antibody according to the first or second aspect of the invention. Furthermore, in a fourth aspect an expression cassette or vector comprising the nucleic acid according to the invention and a promoter operatively connected with said nucleic acid and, in a fifth aspect, a host cell comprising the nucleic acid or the expression cassette or vector according to the invention are provided.

In a sixth aspect, the present invention provides a conjugate comprising the antibody according to the first or second aspect of the invention conjugated to a further agent.

In a seventh aspect, the present invention is directed to a composition comprising the antibody according to the first or second aspect of the invention, the nucleic acid according to the third aspect of the invention, the expression cassette or vector according to the fourth aspect of the invention, the host cell according to the fifth aspect of the invention, or the conjugate according to the sixth aspect of the invention.

According to an eighth aspect, the invention provides the antibody, the nucleic acid, the expression cassette or vector, the host cell, the composition or the conjugate according to the invention for use in medicine, in particular in the treatment of cancer.

Other objects, features, advantages and aspects of the present invention will become apparent to those skilled in the art from the following description and appended claims. It should be understood, however, that the following description, appended claims, and specific examples, which indicate preferred embodiments of the application, are given by way of illustration only. Various changes and modifications within the spirit and scope of the disclosed invention will become readily apparent to those skilled in the art from reading the following.

### DEFINITIONS

As used herein, the following expressions are generally intended to preferably have the meanings as set forth below, except to the extent that the context in which they are used indicates otherwise.

The expression “comprise”, as used herein, besides its literal meaning also includes and specifically refers to the expressions “consist essentially of” and “consist of”. Thus, the expression “comprise” refers to embodiments wherein the subject-matter which “comprises” specifically listed elements does not comprise further elements as well as embodiments wherein the subject-matter which “comprises” specifically listed elements may and/or indeed does encompass further elements. Likewise, the expression “have” is to be understood as the expression “comprise”, also including and specifically referring to the expressions “consist essentially of” and “consist of”. The term “consist essentially of”, where possible, in particular refers to embodiments wherein the subject-matter comprises 20% or less, in particular 15% or less, 10% or less or especially 5% or less further elements in addition to the specifically listed elements of which the subject-matter consists essentially of.

The term “antibody” in particular refers to a protein comprising at least two heavy chains and two light chains connected by disulfide bonds. Each heavy chain is comprised of a heavy chain variable region (VH) and a heavy chain constant region (CH). Each light chain is comprised of a light chain variable region (VL) and a light chain constant region (CL). The heavy chain-constant region comprises three or—in the case of antibodies of the IgM- or IgE-type—four heavy chain-constant domains (CH1, CH2, CH3 and CH4) wherein the first constant domain CH1 is adjacent to the variable region and may be connected to the second constant domain CH2 by a hinge region. The light chain-constant region consists only of one constant domain. The variable regions can be further subdivided into regions of hypervariability, termed complementarity determining regions (CDRs), interspersed with regions that are more conserved, termed framework regions (FR), wherein each variable region comprises three CDRs and four FRs. The amino acid residues of the CDRs are in particular determined based on the CDR localization according to the IMGT system.

The variable regions of the heavy and light chains contain a binding domain that interacts with an antigen. The heavy chain constant regions may be of any type such as γ-, δ-, α-, μ- or ε-type heavy chains. Preferably, the heavy chain of the antibody is a γ-chain. Furthermore, the light chain constant region may also be of any type such as κ- or λ-type light chains. Preferably, the light chain of the antibody is a κ-chain. The terms “γ- (δ-, α-, μ- or ε-) type heavy chain” and “κ- (λ-) type light chain” refer to antibody heavy chains or antibody light chains, respectively, which have constant region amino acid sequences derived from naturally occurring heavy or light chain constant region amino acid sequences, especially human heavy or light chain constant region amino acid sequences. In particular, the amino acid sequence of the constant domains of a γ-type (especially γ1-type) heavy chain is at least 95%, especially at least 98%, identical to the amino acid sequence of the constant domains of a human γ (especially one of the allotypes of the human γ1) antibody heavy chain. Furthermore, the amino acid sequence of the constant domain of a κ-type light chain is in particular at least 95%, especially at least 98%, identical to the amino acid sequence of the constant domain of one of the allotypes of the human κ antibody light chain. The constant regions of the antibodies may mediate the binding of the immunoglobulin to host tissues or factors, including various cells of the immune system (e.g., effector cells) and the first component (C1q) of the classical complement system. The antibody can be e.g. a humanized, human or chimeric antibody.

The antigen-binding portion of an antibody usually refers to full length or one or more fragments of an antibody that retains the ability to specifically bind to an antigen. It has been shown that the antigen-binding function of an antibody can be performed by fragments of a full-length antibody. Examples of binding fragments of an antibody include a Fab fragment, a monovalent fragment consisting of the VL, VH, CL and CH1 domains; a F(ab)₂ fragment, a bivalent fragment comprising two Fab fragments, each of which binds to the same antigen, linked by a disulfide bridge at the hinge region; a Fd fragment consisting of the VH and CH1 domains; a Fv fragment consisting of the VL and VH domains of a single arm of an antibody; and a dAb fragment, which consists of a VH domain.

The “Fab part” of an antibody in particular refers to a part of the antibody comprising the heavy and light chain variable regions (VH and VL) and the first domains of the heavy and light chain constant regions (CH1 and CL). In cases where the antibody does not comprise all of these regions, then the term “Fab part” only refers to those of the regions VH, VL, CH1 and CL which are present in the antibody. Preferably, “Fab part” refers to that part of an antibody corresponding to the fragment obtained by digesting a natural antibody with papain which contains the antigen binding activity of the antibody. In particular, the Fab part of an antibody encompasses the antigen binding site or antigen binding ability thereof. Preferably, the Fab part comprises at least the V_{H} region of the antibody.

The “Fc part” of an antibody in particular refers to a part of the antibody comprising the heavy chain constant regions 2, 3 and—where applicable—4 (CH2, CH3 and CH4). In particular, the Fc part comprises two of each of these regions. In cases where the antibody does not comprise all of these regions, then the term “Fc part” only refers to those of the regions CH2, CH3 and CH4 which are present in the antibody. Preferably, the Fc part comprises at least the CH2 region of the antibody. Preferably, “Fc part” refers to that part of an antibody corresponding to the fragment obtained by digesting a natural antibody with papain which does not contain the antigen binding activity of the antibody. In particular, the Fc part of an antibody is capable of binding to the Fc receptor and thus, e.g. comprises an Fc receptor binding site or an Fc receptor binding ability.

According to the present invention, the term “chimeric antibody” in particular refers to an antibody wherein the constant regions are derived from a human antibody or a human antibody consensus sequence, and wherein at least one and preferably both variable regions are derived from a non-human antibody, e.g. from a rodent antibody such as a mouse antibody.

According to the present invention, the term “humanized antibody” in particular refers to a non-human antibody comprising human constant regions and variable regions which amino acid sequences are modified so as to reduce the immunogenicity of the antibody when administered to the human body. An exemplary method for constructing humanized antibodies is CDR grafting, wherein the CDRs or the specificity determining residues (SDRs) of a non-human antibody are combined with human-derived framework regions. Optionally, some residues of the human framework regions may be backmutated towards the residues of the parent non-human antibody, e.g. for increasing or restoring the antigen binding affinity. Other humanization methods include, for example, resurfacing, superhumanization, and human string content optimization. In the resurfacing methods, only those residues of the non-human framework regions which are positioned at the surface of the antibody are replaced by residues present in corresponding human antibody sequences at said position. Superhumanization essentially corresponds to CDR grafting. However, while during CDR grafting the human framework regions are normally chosen based on their homology to the non-human framework regions, in superhumanization it is the similarity of the CDRs on the basis of which the human framework regions are chosen. In the human string content optimization the differences of the non-human antibody sequence to the human germline sequences is scored and then the antibody is mutated to minimize said score. Furthermore, humanized antibodies can also be obtained by empirical methods wherein large libraries of human framework regions or human antibodies are used to generate multiple antibody humanized candidates and then the most promising candidate is determined by screening methods. Also with the above-described rational approaches several humanized antibody candidates can be generated and then screened, for example for their antigen binding.

The term “human antibody”, as used herein, is intended to include antibodies having variable regions in which both the framework and CDR regions are derived from sequences of human origin.

The term “antibody”, as used herein, refers in certain embodiments to a population of antibodies of the same kind. In particular, all antibodies of the population of the antibody exhibit the features used for defining the antibody. In certain embodiments, all antibodies in the population of the antibody have the same amino acid sequence. Reference to a specific kind of antibody, such as an anti-LYPD3 antibody, in particular refers to a population of this kind of antibody.

The term “antibody” as used herein includes the full-length antibody as well as fragments and derivatives of said antibody. A “fragment or derivative” of an antibody in particular is a protein or glycoprotein which is derived from said antibody and is capable of binding to the same antigen, in particular to the same epitope as the antibody. Thus, a fragment or derivative of an antibody herein generally refers to a functional fragment or derivative. In particularly preferred embodiments, the fragment or derivative of an antibody comprises a heavy chain variable region. It has been shown that the antigen-binding function of an antibody can be performed by fragments of a full-length antibody or derivatives thereof. Examples of fragments of an antibody include (i) Fab fragments, monovalent fragments consisting of the variable region and the first constant domain of each the heavy and the light chain; (ii) F(ab)₂ fragments, bivalent fragments comprising two Fab fragments linked by a disulfide bridge at the hinge region; (iii) Fd fragments consisting of the variable region and the first constant domain CH1 of the heavy chain; (iv) Fv fragments consisting of the heavy chain and light chain variable region of a single arm of an antibody; (v) scFv fragments, Fv fragments consisting of a single polypeptide chain; (vi) (Fv)₂ fragments consisting of two Fv fragments covalently linked together; (vii) a heavy chain variable domain; and (viii) multibodies consisting of a heavy chain variable region and a light chain variable region covalently linked together in such a manner that association of the heavy chain and light chain variable regions can only occur intermolecular but not intramolecular. Derivatives of an antibody in particular include antibodies which bind to the same antigen as the parent antibody, but which have a different amino acid sequence than the parent antibody from which it is derived. These antibody fragments and derivatives are obtained using conventional techniques known to those with skill in the art.

A target amino acid sequence is “derived” from or “corresponds” to a reference amino acid sequence if the target amino acid sequence shares a homology or identity over its entire length with a corresponding part of the reference amino acid sequence of at least 75%, more preferably at least 80%, at least 85%, at least 90%, at least 93%, at least 95%, at least 97%, at least 98% or at least 99%. The “corresponding part” means that, for example, framework region 1 of a heavy chain variable region (FRH1) of a target antibody corresponds to framework region 1 of the heavy chain variable region of the reference antibody. In particular embodiments, a target amino acid sequence which is “derived” from or “corresponds” to a reference amino acid sequence is 100% homologous, or in particular 100% identical, over its entire length with a corresponding part of the reference amino acid sequence. A “homology” or “identity” of an amino acid sequence or nucleotide sequence is preferably determined according to the invention over the entire length of the reference sequence or over the entire length of the corresponding part of the reference sequence which corresponds to the sequence which homology or identity is defined. An antibody derived from a parent antibody which is defined by one or more amino acid sequences, such as specific CDR sequences or specific variable region sequences, in particular is an antibody having amino acid sequences, such as CDR sequences or variable region sequences, which are at least 75%, preferably at least 80%, at least 85%, at least 90%, at least 93%, at least 95%, at least 97%, at least 98% or at least 99% homologous or identical, especially identical, to the respective amino acid sequences of the parent antibody. In certain embodiments, the antibody derived from (i.e. derivative of) a parent antibody comprises the same CDR sequences as the parent antibody, but differs in the remaining sequences of the variable regions.

The term “antibody” as used herein also refers to multivalent and multispecific antibodies, i.e. antibody constructs which have more than two binding sites each binding to the same epitope and antibody constructs which have one or more binding sites binding to a first epitope and one or more binding sites binding to a second epitope, and optionally even further binding sites binding to further epitopes.

“Specific binding” preferably means that an agent such as an antibody binds stronger to a target such as an epitope for which it is specific compared to the binding to another target. An agent binds stronger to a first target compared to a second target if it binds to the first target with a dissociation constant (K_{d}) which is lower than the dissociation constant for the second target. Preferably the dissociation constant for the target to which the agent binds specifically is more than 10-fold, 30-fold, 100-fold or more than 500-fold lower than the dissociation constant for the target to which the agent does not bind specifically. Furthermore, the term “specific binding” in particular indicates a binding affinity between the binding partners with an affinity constant Kₐ of at least 10⁵ M⁻¹, preferably at least 10⁶ M⁻¹, more preferably at least 10⁷ M⁻¹, for example at least 10⁸ M⁻¹. An antibody specific for a certain antigen in particular refers to an antibody which is capable of binding to said antigen with an affinity having a Kₐ of at least 10⁵ M⁻¹, preferably at least 10⁶ M⁻¹, more preferably at least 10⁷ M⁻¹. For example, the term “anti- LYPD3 antibody” refers to an antibody specifically binding LYPD3 and preferably is capable of binding to LYPD3 with an affinity having a Kₐ of at least 10⁵ M⁻¹, preferably at least 10⁶ M⁻¹, more preferably at least 10⁷ M⁻¹. The term “binding” as used herein in particular refers to specific binding.

The term “epitope” as used herein refers to the amino acid residues and glycan structures on the antigen of an antibody which are either directly contacted by the amino acids of the antibody, in particular the amino acids of the CDRs of the antibody, or which are in direct vicinity thereof and influence the binding of the antibody to its antigen.

The term “LYPD3” according to the present invention in particular refers to the human LYPD3 protein, especially the mature human LYPD3 protein. LYPD3 as used herein especially refers to the human LYPD3 protein according to the UniProt entry O95274.LYPD3 in particular comprises, especially consists of, the amino acid sequence of positions 31 to 326 of SEQ ID NO: 137, or an amino acid sequence which is at least 90%, especially at least 95% identical to positions 31 to 326 of SEQ ID NO: 137 over the entire length. LYPD3 in particular is post-translationally modified and may carry O-glycosylation, N-glycosylation and/or a glycosylphosphatidylinositol (GPI) anchor. For example, LYPD3 may carry O-glycosylations at one or more positions corresponding to Ser223, Ser232, Thr247, Thr248, Ser251, Thr252, Thr253, Ser254, Thr256, Thr257, Ser258, Thr259, Ser260, Thr266, Ser267, Thr268, Thr269, Thr276, Ser277, Thr279, Ser289, Thr297, Ser307 and Ser309 of SEQ ID NO: 137.

The term “GalNAcα1-”, also called “Tn”, “Tn antigen” or “Thomsen nouvelle antigen” refers to a monosaccharide structure consisting of an N-acetyl galactosaminyl residue attached via an α-glycosidic bond to a supporting structure, especially to a serine or threonine residue of a protein or peptide.

The term “sialylated GalNAcα1-”, also called “sTn”, “sialylated Tn antigen”, or “sialylated Thomsen nouvelle antigen” refers to a disaccharide structure consisting of an N-acetyl galactosaminyl residue attached via an α-glycosidic bond to a supporting structure, especially to a serine or threonine residue of a protein or peptide. To this structure, a sialic acid residue is attached via α2-6 bond to the N-acetyl galactosaminyl residue resulting in the disaccharide structure Siaα2-6GalNAcα1-.

The term “Galβ1-3GalNAcα1-”, also called “TF”, “TF antigen”, “T antigen” or “Thomsen Friedenreich antigen” refers to a disaccharide structure consisting of a galactosyl residue attached via a β1-3 bond to an N-acetyl galactosaminyl residue attached via an α1-glycosidic bond to a supporting structure, especially to a serine or threonine residue of a protein or peptide.

The term “sialylated Galβ1-3GalNAcα1-”, also called “sTF”, “sTF antigen”, “sialylated TF antigen”, “sialylated T antigen” or “sialylated Thomsen Friedenreich antigen” refers to tri- or tetrasaccharide structures consisting of a galactosyl residue attached via a β1-3 bond to an N-acetyl galactosaminyl residue attached via an α1-glycosidic bond to a supporting structure, especially to a serine or threonine residue of a protein or peptide. To this structure, a sialic acid residue is attached via an α2-3 bond to the galactosyl residue and/or a sialic acid residue is attached via an α2-6 bond to the N-acetyl galactosaminyl residue resulting in the trisaccharide structures Siaα2-3Galβ1-3GalNAcα1- and Galβ1-3(Siaα2-6)GalNAcα1- or the tetrasaccharide structure Siaα2-6(Siaα2-3Galβ1-3)GalNAcα1-.

In the above indicated structures, Gal represents a galactose residue and GalNAc represents an N-acetyl galactosamine residue. “β1-3”, “α2-3” and “α2-6” indicate the linkage of the two adjacent monosaccharide residues, especially between carbon atom C1 or C2, respectively, of the left monosaccharide and carbon atom C3 or C6, respectively, of the right monosaccharide, wherein the linkage can be in α- or β-position (shown in the following scheme for glucose):

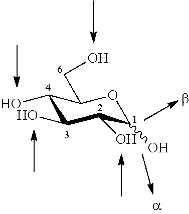

The term “GalNAcα1-” indicates that the GalNAc residue at the reducing end of the oligosaccharide is linked via its carbon atom C1 in α-configuration to the support structure.

The term “sialic acid” in particular refers to any N- or O-substituted derivatives of neuraminic acid. It may refer to both 5-N-acetylneuraminic acid (NeuNAc) and 5-N-glycolylneuraminic acid (NeuGc), but preferably only refers to 5-N-acetylneuraminic acid.

The terms “glycan”, “glycan structure”, “carbohydrate”, “carbohydrate chain” and “carbohydrate structure” are generally used synonymously herein.

In a “conjugate” two or more compounds are linked together. In certain embodiments, at least some of the properties from each compound are retained in the conjugate. Linking may be achieved by a covalent or non-covalent bond. Preferably, the compounds of the conjugate are linked via a covalent bond. The different compounds of a conjugate may be directly bound to each other via one or more covalent bonds between atoms of the compounds. Alternatively, the compounds may be bound to each other via a chemical moiety such as a linker molecule wherein the linker is covalently attached to atoms of the compounds. If the conjugate is composed of more than two compounds, then these compounds may, for example, be linked in a chain conformation, one compound attached to the next compound, or several compounds each may be attached to one central compound.

The term “nucleic acid” includes single-stranded and double-stranded nucleic acids and ribonucleic acids as well as deoxyribonucleic acids. It may comprise naturally occurring as well as synthetic nucleotides and can be naturally or synthetically modified, for example by methylation, 5′- and/or 3′-capping.

The term “expression cassette” in particular refers to a nucleic acid construct which is capable of enabling and regulating the expression of a coding nucleic acid sequence introduced therein. An expression cassette may comprise promoters, ribosome binding sites, enhancers and other control elements which regulate transcription of a gene or translation of an mRNA. The exact structure of expression cassette may vary as a function of the species or cell type, but generally comprises 5′-untranscribed and 5′- and 3′-untranslated sequences which are involved in initiation of transcription and translation, respectively, such as TATA box, capping sequence, CAAT sequence, and the like. More specifically, 5′-untranscribed expression control sequences comprise a promoter region which includes a promoter sequence for transcriptional control of the operatively connected nucleic acid. Expression cassettes may also comprise enhancer sequences or upstream activator sequences.

According to the invention, the term “promoter” refers to a nucleic acid sequence which is located upstream (5′) of the nucleic acid sequence which is to be expressed and controls expression of the sequence by providing a recognition and binding site for RNA-polymerases. The “promoter” may include further recognition and binding sites for further factors which are involved in the regulation of transcription of a gene. A promoter may control the transcription of a prokaryotic or eukaryotic gene. Furthermore, a promoter may be “inducible”, i.e. initiate transcription in response to an inducing agent, or may be “constitutive” if transcription is not controlled by an inducing agent. A gene which is under the control of an inducible promoter is not expressed or only expressed to a small extent if an inducing agent is absent. In the presence of the inducing agent the gene is switched on or the level of transcription is increased. This is mediated, in general, by binding of a specific transcription factor.

The term “vector” is used here in its most general meaning and comprises any intermediary vehicle for a nucleic acid which enables said nucleic acid, for example, to be introduced into prokaryotic and/or eukaryotic cells and, where appropriate, to be integrated into a genome. Vectors of this kind are preferably replicated and/or expressed in the cells. Vectors comprise plasmids, phagemids, bacteriophages or viral genomes. The term “plasmid” as used herein generally relates to a construct of extrachromosomal genetic material, usually a circular DNA duplex, which can replicate independently of chromosomal DNA.

According to the invention, the term “host cell” relates to any cell which can be transformed or transfected with an exogenous nucleic acid. The term “host cells” comprises according to the invention prokaryotic (e.g. E. coli) or eukaryotic cells (e.g. mammalian cells, in particular human or hamster cells, yeast cells and insect cells). Particular preference is given to mammalian cells such as cells from humans, mice, hamsters, pigs, goats, or primates. The cells may be derived from a multiplicity of tissue types and comprise primary cells and cell lines. A nucleic acid may be present in the host cell in the form of a single copy or of two or more copies and, in one embodiment, is expressed in the host cell.

The term “patient” means according to the invention a human being, a nonhuman primate or another animal, in particular a mammal such as a cow, horse, pig, sheep, goat, dog, cat or a rodent such as a mouse and rat. In a particularly preferred embodiment, the patient is a human being.

The term “cancer” according to the invention in particular comprises leukemias, seminomas, melanomas, teratomas, lymphomas, neuroblastomas, gliomas, rectal cancer, endometrial cancer, kidney cancer, adrenal cancer, thyroid cancer, blood cancer, skin cancer, cancer of the brain, cervical cancer, intestinal cancer, liver cancer, colon cancer, stomach cancer, intestine cancer, head and neck cancer, gastrointestinal cancer, lymph node cancer, esophagus cancer, colorectal cancer, pancreas cancer, ear, nose and throat (ENT) cancer, bladder cancer, breast cancer, prostate cancer, cancer of the uterus, ovarian cancer and lung cancer and the metastases thereof. The term cancer according to the invention also comprises cancer metastases. The term cancer further also refers to and/or includes cancer stem cells, especially the cancer stem cells of the specific types of cancer listed above.

By “tumor” is meant a group of cells or tissue that is formed by misregulated cellular proliferation. Tumors may show partial or complete lack of structural organization and functional coordination with the normal tissue, and usually form a distinct mass of tissue, which may be either benign or malignant.

By “metastasis” is meant the spread of cancer cells from its original site to another part of the body. The formation of metastasis is a very complex process and normally involves detachment of cancer cells from a primary tumor, entering the body circulation and settling down to grow within normal tissues elsewhere in the body. When tumor cells metastasize, the new tumor is called a secondary or metastatic tumor, and its cells normally resemble those in the original tumor. This means, for example, that, if breast cancer metastasizes to the lungs, the secondary tumor is made up of abnormal breast cells, not of abnormal lung cells. The tumor in the lung is then called metastatic breast cancer, not lung cancer.

The term “pharmaceutical composition” particularly refers to a composition suitable for administering to a human or animal, i.e., a composition containing components which are pharmaceutically acceptable. Preferably, a pharmaceutical composition comprises an active compound or a salt or prodrug thereof together with a carrier, diluent or pharmaceutical excipient such as buffer, preservative and tonicity modifier.

### DETAILED DESCRIPTION OF THE INVENTION

The present invention is based on the development of anti-LYPD3 antibodies which specifically bind tumor-associated LYPD3. These antibodies were generated using LYPD3 or LYPD3 fragments carrying O-glycan structures produced by cancer cells and selecting those antibodies which bind to LYPD3 in an O-glycosylation-dependent manner. O-glycosylation produced by normal cells significantly differs from O-glycosylation produced by cancer cells. For example, normal cells produce large O-glycan structures while LYPD3 on tumor cells predominantly carries mono-, di- and trisaccharides such as GalNAcα1- (Tn), sialylated GalNAcα1- (sTn), Galβ1-3GalNAcα1- (TF) and sialylated Galβ1-3GalNAcα1- (sTF). The antibodies according to the invention recognize and bind to these small O-glycan structures of tumor-associated LYPD3. Therefore, these antibodies bind to LYPD3 on tumor cells with a higher affinity than to LYPD3 of normal tissue and are able to discriminate between tumor-associated and non-tumor-associated LYPD3.

### 1. Anti-LYPD3 Antibodies

In view of these findings, the present invention provides in a first aspect an antibody which is capable of specifically binding to glycosylated human LYPD3 at an epitope comprising an oligosaccharide structure selected from the group consisting of GalNAcα1-, sialylated GalNAcα1-, Galβ1-3GalNAcα1-, and sialylated Galβ1-3GalNAcα1-, which is attached to a serine or threonine residue of LYPD3. Preferably, the antibody is capable of specifically binding to glycosylated human LYPD3 at an epitope comprising one or more GalNAcα1- and/or Galβ1-3GalNAcα1- oligosaccharide structures which are attached to serine and/or threonine residues of LYPD3.

An antibody capable of specifically binding to glycosylated human LYPD3 binds to glycosylated human LYPD3 with a higher binding affinity compared to non-glycosylated human LYPD3. In certain embodiments, an antibody capable of specifically binding to glycosylated human LYPD3 binds to glycosylated human LYPD3 with a higher binding affinity compared to an unrelated protein carrying the same glycan structures. The term “a higher binding affinity” as used herein in particular refers to a difference in the dissociation constant of at least 10-fold, especially at least 25-fold, in particular at least 100-fold.

In certain embodiments, the antibody is capable of binding to human LYPD3 at an epitope comprising more than one oligosaccharide structure, for example 2, 3 or 4 oligosaccharide structures, which are attached to serine and/or threonine residues of LYPD3. One or more of these further oligosaccharide structures may also be selected from the group consisting of GalNAcα1-, sialylated GalNAcα1-, Galβ1-3GalNAcα1-, and sialylated Galβ1-3GalNAcα1-. In specific embodiments, the antibody is capable of binding to human LYPD3 at an epitope comprising two oligosaccharide structures, each selected from the group consisting of GalNAcα1-, sialylated GalNAcα1-, Galβ1-3GalNAcα1-, and sialylated Galβ1-3GalNAcα1-, and each being attached to a serine or threonine residue of LYPD3. Especially, the antibody is capable of binding to human LYPD3 at an epitope comprising three oligosaccharide structures, each selected from the group consisting of GalNAcα1-, sialylated GalNAcα1-, Galβ1-3GalNAcα1-, and sialylated Galβ1-3GalNAcα1-, and each being attached to a serine or threonine residue of LYPD3.

The one or more oligosaccharide structures are in particular present in the C terminal Ser/Thr/Pro-rich domain of LYPD3. In certain embodiments, at least one and especially all of the oligosaccharide structures are attached to a serine or threonine residue within positions 234 to 303 of SEQ ID NO: 137. In particular, at least one and especially all of the oligosaccharide structures are attached to a serine or threonine residue within positions 247 to 297 of SEQ ID NO: 137.

The antibody is capable of binding to human LYPD3 at a combined peptide and carbohydrate epitope. For example, the epitope comprises one or more amino acids within positions 234 to 303 of SEQ ID NO: 137. In particular, the epitope comprises one or more amino acids within positions 247 to 297 of SEQ ID NO: 137. In specific embodiments, at least 25%, especially at least 50%, in particular at least 75% of the amino acids of the epitope are present within positions 234 to 303 of SEQ ID NO: 137, in particular within positions 247 to 297 of SEQ ID NO: 137. Preferably, all of the amino acids of the epitope are present within positions 234 to 303 of SEQ ID NO: 137.

The term “capable of binding to human LYPD3 at a combined peptide and carbohydrate epitope” in this respect means that the antibody either directly interacts with amino acids and one or more glycan structures of LYPD3, or directly interacts only with amino acids of LYPD3, or directly interacts only with carbohydrate chains of LYPD3. In all cases, the binding affinity of the antibody to LYPD3 is higher if an oligosaccharide structure selected from the group consisting of GalNAcα1-, sialylated GalNAcα1-, Galβ1-3GalNAcα1-, and sialylated Galβ1-3GalNAcα1- is attached to the amino acids of LYPD3 compared to non-glycosylated LYPD3. In embodiments where the antibody directly interacts only with amino acids of LYPD3, but not with carbohydrate chains, the higher affinity for the O-glycosylated LYPD3 is based on conformational changes of the polypeptide chain induced by the oligosaccharides attached to LYPD3.

In certain embodiments, the antibody specifically binds to tumor-associated LYPD3. The antibody especially binds to glycosylated human LYPD3 with a higher binding affinity compared to an oligosaccharide structure selected from the group consisting of GalNAcα1-, sialylated GalNAcα1-, Galβ1-3GalNAcα1-, and sialylated Galβ1-3GalNAcα1-, which is attached to a carrier molecule unrelated to LYPD3. For example, the carrier molecule may be polyacrylamide (PAA) or a random peptide. In certain embodiments, the antibody is capable of binding tumor-associated LYPD3 with a higher binding affinity than LYPD3 expressed by cells of normal tissue.

In specific embodiments, the antibody is capable of binding to human LYPD3 glycosylated with any one or more of GalNAcα1-, sialylated GalNAcα1-, Galβ1-3GalNAcα1-, and sialylated Galβ1-3GalNAcα1-. Thus, the antibody binds to LYPD3 if it is glycosylated with GalNAcα1-, sialylated GalNAcα1-, Galβ1-3GalNAcα1-, sialylated Galβ1-3GalNAcα1-, or any mixture of two or all of these glycan structures. In these embodiments, LYPD3 may also carry other glycan structures as long as at least one of the above glycan structures is also present. In particular, these antibodies specifically bind to human LYPD3 glycosylated with any one or more of GalNAcα1-, sialylated GalNAcα1-, Galβ1-3GalNAcα1-, and sialylated Galβ1-3GalNAcα1-.

In specific embodiments, the antibody is capable of binding to human LYPD3 glycosylated with any one or both of GalNAcα1- and Galβ1-3GalNAcα1-. Thus, the antibody binds to LYPD3 if it is glycosylated with GalNAcα1-, Galβ1-3GalNAcα1-, or any mixture of these two glycan structures. In these embodiments, LYPD3 may also carry other glycan structures as long as at least one of the above glycan structures is also present. In particular, these antibodies specifically bind to human LYPD3 glycosylated with any one or both of GalNAcα1- and Galβ1-3GalNAcα1-.

In specific embodiments, the antibody is capable of binding to human LYPD3 glycosylated with any one or both of Galβ1-3GalNAcα1- and sialylated Galβ1-3GalNAcα1-. Thus, the antibody binds to LYPD3 if it is glycosylated with Galβ1-3GalNAcα1-, sialylated Galβ1-3GalNAcα1-, or any mixture of these two glycan structures. In these embodiments, LYPD3 may also carry other glycan structures as long as at least one of the above glycan structures is also present. In particular, these antibodies specifically bind to human LYPD3 glycosylated with any one or both of Galβ1-3GalNAcα1- and sialylated Galβ1-3GalNAcα1-.

In specific embodiments, the antibody is capable of binding to human LYPD3 glycosylated with Galβ1-3GalNAcα1-. Thus, the antibody binds to LYPD3 if it is glycosylated with Galβ1-3GalNAcα1-. In these embodiments, LYPD3 may also carry other glycan structures as long as Galβ1-3GalNAcα1- is also present. In particular, these antibodies specifically bind to human LYPD3 glycosylated with Galβ1-3GalNAcα1-.

In a second aspect, the present invention provides an anti-LYPD3 antibody which comprises
(i) a heavy chain variable region comprising the complementarity-determining regions (CDRs) CDR-H1 having the amino acid sequence of SEQ ID NO: 1, CDR-H2 having the amino acid sequence of SEQ ID NO: 2 and CDR-H3 having the amino acid sequence of SEQ ID NO: 3, and
   a light chain variable region comprising the complementarity-determining regions (CDRs) CDR-L1 having the amino acid sequence of SEQ ID NO: 4, CDR-L2 having the amino acid sequence of SEQ ID NO: 5 and CDR-L3 having the amino acid sequence of SEQ ID NO: 6, or
(ii) a heavy chain variable region comprising the complementarity-determining regions (CDRs) CDR-H1 having the amino acid sequence of SEQ ID NO: 9, CDR-H2 having the amino acid sequence of SEQ ID NO: 10 and CDR-H3 having the amino acid sequence of SEQ ID NO: 11, and
   a light chain variable region comprising the complementarity-determining regions (CDRs) CDR-L1 having the amino acid sequence of SEQ ID NO: 12, CDR-L2 having the amino acid sequence of SEQ ID NO: 13 and CDR-L3 having the amino acid sequence of SEQ ID NO: 14; or
(iii) a heavy chain variable region comprising the complementarity-determining regions (CDRs) CDR-H1 having the amino acid sequence of SEQ ID NO: 17, CDR-H2 having the amino acid sequence of SEQ ID NO: 18 and CDR-H3 having the amino acid sequence of SEQ ID NO: 19, and
   a light chain variable region comprising the complementarity-determining regions (CDRs) CDR-L1 having the amino acid sequence of SEQ ID NO: 20, CDR-L2 having the amino acid sequence of SEQ ID NO: 21 and CDR-L3 having the amino acid sequence of SEQ ID NO: 22; or
(iv) a heavy chain variable region comprising the complementarity-determining regions (CDRs) CDR-H1 having the amino acid sequence of SEQ ID NO: 25, CDR-H2 having the amino acid sequence of SEQ ID NO: 26 and CDR-H3 having the amino acid sequence of SEQ ID NO: 27, and
   a light chain variable region comprising the complementarity-determining regions (CDRs) CDR-L1 having the amino acid sequence of SEQ ID NO: 28, CDR-L2 having the amino acid sequence of SEQ ID NO: 29 and CDR-L3 having the amino acid sequence of SEQ ID NO: 30; or
(v) a heavy chain variable region comprising the complementarity-determining regions (CDRs) CDR-H1 having the amino acid sequence of SEQ ID NO: 33, CDR-H2 having the amino acid sequence of SEQ ID NO: 34 and CDR-H3 having the amino acid sequence of SEQ ID NO: 35, and
   a light chain variable region comprising the complementarity-determining regions (CDRs) CDR-L1 having the amino acid sequence of SEQ ID NO: 36, CDR-L2 having the amino acid sequence of SEQ ID NO: 37 and CDR-L3 having the amino acid sequence of SEQ ID NO: 38; or
(vi) a heavy chain variable region comprising the complementarity-determining regions (CDRs) CDR-H1 having the amino acid sequence of SEQ ID NO: 41, CDR-H2 having the amino acid sequence of SEQ ID NO: 42 and CDR-H3 having the amino acid sequence of SEQ ID NO: 43, and
   a light chain variable region comprising the complementarity-determining regions (CDRs) CDR-L1 having the amino acid sequence of SEQ ID NO: 44, CDR-L2 having the amino acid sequence of SEQ ID NO: 45 and CDR-L3 having the amino acid sequence of SEQ ID NO: 46; or
(vii) a heavy chain variable region comprising the complementarity-determining regions (CDRs) CDR-H1 having the amino acid sequence of SEQ ID NO: 49, CDR-H2 having the amino acid sequence of SEQ ID NO: 50 and CDR-H3 having the amino acid sequence of SEQ ID NO: 51, and
   a light chain variable region comprising the complementarity-determining regions (CDRs) CDR-L1 having the amino acid sequence of SEQ ID NO: 52, CDR-L2 having the amino acid sequence of SEQ ID NO: 53 and CDR-L3 having the amino acid sequence of SEQ ID NO: 54; or
(viii) a heavy chain variable region comprising the complementarity-determining regions (CDRs) CDR-H1 having the amino acid sequence of SEQ ID NO: 57, CDR-H2 having the amino acid sequence of SEQ ID NO: 58 and CDR-H3 having the amino acid sequence of SEQ ID NO: 59, and
   a light chain variable region comprising the complementarity-determining regions (CDRs) CDR-L1 having the amino acid sequence of SEQ ID NO: 60, CDR-L2 having the amino acid sequence of SEQ ID NO: 61 and CDR-L3 having the amino acid sequence of SEQ ID NO: 62; or
(ix) a heavy chain variable region comprising the complementarity-determining regions (CDRs) CDR-H1 having the amino acid sequence of SEQ ID NO: 65, CDR-H2 having the amino acid sequence of SEQ ID NO: 66 and CDR-H3 having the amino acid sequence of SEQ ID NO: 67, and
   a light chain variable region comprising the complementarity-determining regions (CDRs) CDR-L1 having the amino acid sequence of SEQ ID NO: 68, CDR-L2 having the amino acid sequence of SEQ ID NO: 69 and CDR-L3 having the amino acid sequence of SEQ ID NO: 70; or
(x) a heavy chain variable region comprising the complementarity-determining regions (CDRs) CDR-H1 having the amino acid sequence of SEQ ID NO: 73, CDR-H2 having the amino acid sequence of SEQ ID NO: 74 and CDR-H3 having the amino acid sequence of SEQ ID NO: 75, and
   a light chain variable region comprising the complementarity-determining regions (CDRs) CDR-L1 having the amino acid sequence of SEQ ID NO: 76, CDR-L2 having the amino acid sequence of SEQ ID NO: 77 and CDR-L3 having the amino acid sequence of SEQ ID NO: 78; or
(xi) a heavy chain variable region comprising the complementarity-determining regions (CDRs) CDR-H1 having the amino acid sequence of SEQ ID NO: 81, CDR-H2 having the amino acid sequence of SEQ ID NO: 82 and CDR-H3 having the amino acid sequence of SEQ ID NO: 83, and
   a light chain variable region comprising the complementarity-determining regions (CDRs) CDR-L1 having the amino acid sequence of SEQ ID NO: 84, CDR-L2 having the amino acid sequence of SEQ ID NO: 85 and CDR-L3 having the amino acid sequence of SEQ ID NO: 86; or
(xii) a heavy chain variable region comprising the complementarity-determining regions (CDRs) CDR-H1 having the amino acid sequence of SEQ ID NO: 89, CDR-H2 having the amino acid sequence of SEQ ID NO: 90 and CDR-H3 having the amino acid sequence of SEQ ID NO: 91, and
   a light chain variable region comprising the complementarity-determining regions (CDRs) CDR-L1 having the amino acid sequence of SEQ ID NO: 92, CDR-L2 having the amino acid sequence of SEQ ID NO: 93 and CDR-L3 having the amino acid sequence of SEQ ID NO: 94; or
(xiii) a heavy chain variable region comprising the complementarity-determining regions (CDRs) CDR-H1 having the amino acid sequence of SEQ ID NO: 97, CDR-H2 having the amino acid sequence of SEQ ID NO: 98 and CDR-H3 having the amino acid sequence of SEQ ID NO: 99, and
   a light chain variable region comprising the complementarity-determining regions (CDRs) CDR-L1 having the amino acid sequence of SEQ ID NO: 100, CDR-L2 having the amino acid sequence of SEQ ID NO: 101 and CDR-L3 having the amino acid sequence of SEQ ID NO: 102; or
(xiv) a heavy chain variable region comprising the complementarity-determining regions (CDRs) CDR-H1 having the amino acid sequence of SEQ ID NO: 105, CDR-H2 having the amino acid sequence of SEQ ID NO: 106 and CDR-H3 having the amino acid sequence of SEQ ID NO: 107, and
   a light chain variable region comprising the complementarity-determining regions (CDRs) CDR-L1 having the amino acid sequence of SEQ ID NO: 108, CDR-L2 having the amino acid sequence of SEQ ID NO: 109 and CDR-L3 having the amino acid sequence of SEQ ID NO: 110; or
(xv) a heavy chain variable region comprising the complementarity-determining regions (CDRs) CDR-H1 having the amino acid sequence of SEQ ID NO: 113, CDR-H2 having the amino acid sequence of SEQ ID NO: 114 and CDR-H3 having the amino acid sequence of SEQ ID NO: 115, and
   a light chain variable region comprising the complementarity-determining regions (CDRs) CDR-L1 having the amino acid sequence of SEQ ID NO: 116, CDR-L2 having the amino acid sequence of SEQ ID NO: 117 and CDR-L3 having the amino acid sequence of SEQ ID NO: 118; or
(xvi) a heavy chain variable region comprising the complementarity-determining regions (CDRs) CDR-H1 having the amino acid sequence of SEQ ID NO: 121, CDR-H2 having the amino acid sequence of SEQ ID NO: 122 and CDR-H3 having the amino acid sequence of SEQ ID NO: 123, and
   a light chain variable region comprising the complementarity-determining regions (CDRs) CDR-L1 having the amino acid sequence of SEQ ID NO: 124, CDR-L2 having the amino acid sequence of SEQ ID NO: 125 and CDR-L3 having the amino acid sequence of SEQ ID NO: 126; or
(xvii) a heavy chain variable region comprising the complementarity-determining regions (CDRs) CDR-H1 having the amino acid sequence of SEQ ID NO: 129, CDR-H2 having the amino acid sequence of SEQ ID NO: 130 and CDR-H3 having the amino acid sequence of SEQ ID NO: 131, and
   a light chain variable region comprising the complementarity-determining regions (CDRs) CDR-L1 having the amino acid sequence of SEQ ID NO: 132, CDR-L2 having the amino acid sequence of SEQ ID NO: 133 and CDR-L3 having the amino acid sequence of SEQ ID NO: 134.

In certain embodiments, the anti-LYPD3 antibody comprises a heavy chain variable region and a light chain variable region according to any one or items (i) to (viii), above. In preferred embodiments, the anti-LYPD3 antibody comprises a heavy chain variable region and a light chain variable region according to item (ii), above. In preferred embodiments, the anti-LYPD3 antibody comprises a heavy chain variable region and a light chain variable region according to item (v), above.

In specific embodiments, the anti-LYPD3 antibody may have 1, 2 or 3 amino acid substitutions in total in the six CDR sequences, in particular 1 or 2, especially 1 amino acid substitution. In these embodiments, the anti-LYPD3 antibody retains the antigen specificity of the antibody without said amino acid substitutions. An “amino acid substitution” as used herein also includes an amino acid addition and an amino acid deletion. In certain embodiments, an amino acid substitution is a conservative amino acid substitution.

The antibody according to the second aspect of the present invention in particular exhibits one or more of the binding activities defined for the antibody according to the first aspect. In particular, the antibody according to the second aspect is an antibody according to the first aspect.

In certain embodiments, the antibody according to the invention comprises a heavy chain variable region and a light chain variable region selected from the group consisting of
(i) a heavy chain variable region having an amino acid sequence which is at least 60% identical to the amino acid sequence of SEQ ID NO: 7 over its entire length and comprising the complementarity-determining regions (CDRs) CDR-H1 having the amino acid sequence of SEQ ID NO: 1, CDR-H2 having the amino acid sequence of SEQ ID NO: 2 and CDR-H3 having the amino acid sequence of SEQ ID NO: 3, and
   a light chain variable region having an amino acid sequence which is at least 60% identical to the amino acid sequence of SEQ ID NO: 8 over its entire length and comprising the complementarity-determining regions (CDRs) CDR-L1 having the amino acid sequence of SEQ ID NO: 4, CDR-L2 having the amino acid sequence of SEQ ID NO: 5 and CDR-L3 having the amino acid sequence of SEQ ID NO: 6,
(ii) a heavy chain variable region having an amino acid sequence which is at least 60% identical to the amino acid sequence of SEQ ID NO: 15 over its entire length and comprising CDR-H1 having the amino acid sequence of SEQ ID NO: 9, CDR-H2 having the amino acid sequence of SEQ ID NO: 10 and CDR-H3 having the amino acid sequence of SEQ ID NO: 11, and
   a light chain variable region having an amino acid sequence which is at least 60% identical to the amino acid sequence of SEQ ID NO: 16 over its entire length and comprising CDR-L1 having the amino acid sequence of SEQ ID NO: 12, CDR-L2 having the amino acid sequence of SEQ ID NO: 13 and CDR-L3 having the amino acid sequence of SEQ ID NO: 14;
(iii) a heavy chain variable region having an amino acid sequence which is at least 60% identical to the amino acid sequence of SEQ ID NO: 23 over its entire length and comprising CDR-H1 having the amino acid sequence of SEQ ID NO: 17, CDR-H2 having the amino acid sequence of SEQ ID NO: 18 and CDR-H3 having the amino acid sequence of SEQ ID NO: 19, and
   a light chain variable region having an amino acid sequence which is at least 60% identical to the amino acid sequence of SEQ ID NO: 24 over its entire length and comprising CDR-L1 having the amino acid sequence of SEQ ID NO: 20, CDR-L2 having the amino acid sequence of SEQ ID NO: 21 and CDR-L3 having the amino acid sequence of SEQ ID NO: 22;
(iv) a heavy chain variable region having an amino acid sequence which is at least 60% identical to the amino acid sequence of SEQ ID NO: 31 over its entire length and comprising CDR-H1 having the amino acid sequence of SEQ ID NO: 25, CDR-H2 having the amino acid sequence of SEQ ID NO: 26 and CDR-H3 having the amino acid sequence of SEQ ID NO: 27, and
   a light chain variable region having an amino acid sequence which is at least 60% identical to the amino acid sequence of SEQ ID NO: 32 over its entire length and comprising CDR-L1 having the amino acid sequence of SEQ ID NO: 28, CDR-L2 having the amino acid sequence of SEQ ID NO: 29 and CDR-L3 having the amino acid sequence of SEQ ID NO: 30;
(v) a heavy chain variable region having an amino acid sequence which is at least 60% identical to the amino acid sequence of SEQ ID NO: 39 over its entire length and comprising CDR-H1 having the amino acid sequence of SEQ ID NO: 33, CDR-H2 having the amino acid sequence of SEQ ID NO: 34 and CDR-H3 having the amino acid sequence of SEQ ID NO: 35, and
   a light chain variable region having an amino acid sequence which is at least 60% identical to the amino acid sequence of SEQ ID NO: 40 over its entire length and comprising CDR-L1 having the amino acid sequence of SEQ ID NO: 36, CDR-L2 having the amino acid sequence of SEQ ID NO: 37 and CDR-L3 having the amino acid sequence of SEQ ID NO: 38;
(vi) a heavy chain variable region having an amino acid sequence which is at least 60% identical to the amino acid sequence of SEQ ID NO: 47 over its entire length and comprising CDR-H1 having the amino acid sequence of SEQ ID NO: 41, CDR-H2 having the amino acid sequence of SEQ ID NO: 42 and CDR-H3 having the amino acid sequence of SEQ ID NO: 43, and
   a light chain variable region having an amino acid sequence which is at least 60% identical to the amino acid sequence of SEQ ID NO: 48 over its entire length and comprising CDR-L1 having the amino acid sequence of SEQ ID NO: 44, CDR-L2 having the amino acid sequence of SEQ ID NO: 45 and CDR-L3 having the amino acid sequence of SEQ ID NO: 46;
(vii) a heavy chain variable region having an amino acid sequence which is at least 60% identical to the amino acid sequence of SEQ ID NO: 55 over its entire length and comprising CDR-H1 having the amino acid sequence of SEQ ID NO: 49, CDR-H2 having the amino acid sequence of SEQ ID NO: 50 and CDR-H3 having the amino acid sequence of SEQ ID NO: 51, and
   a light chain variable region having an amino acid sequence which is at least 60% identical to the amino acid sequence of SEQ ID NO: 56 over its entire length and comprising CDR-L1 having the amino acid sequence of SEQ ID NO: 52, CDR-L2 having the amino acid sequence of SEQ ID NO: 53 and CDR-L3 having the amino acid sequence of SEQ ID NO: 54; and
(viii) a heavy chain variable region having an amino acid sequence which is at least 60% identical to the amino acid sequence of SEQ ID NO: 63 over its entire length and comprising CDR-H1 having the amino acid sequence of SEQ ID NO: 57, CDR-H2 having the amino acid sequence of SEQ ID NO: 58 and CDR-H3 having the amino acid sequence of SEQ ID NO: 59, and
   a light chain variable region having an amino acid sequence which is at least 60% identical to the amino acid sequence of SEQ ID NO: 64 over its entire length and comprising the CDR-L1 having the amino acid sequence of SEQ ID NO: 60, CDR-L2 having the amino acid sequence of SEQ ID NO: 61 and CDR-L3 having the amino acid sequence of SEQ ID NO: 62.

In preferred embodiments, the anti-LYPD3 antibody comprises a heavy chain variable region and a light chain variable region according to item (ii), above. In preferred embodiments, the anti-LYPD3 antibody comprises a heavy chain variable region and a light chain variable region according to item (v), above. In the above embodiments the sequence identity may in particular be at least 70, preferably at least 80%, and more preferably at least 90%. The above embodiments in particular are humanized versions of the respective antibodies wherein the changes in the amino acid sequence are substitutions to amino acid residues of a related human antibody sequence.

In certain embodiments, the anti-LYPD3 antibody may additionally have 1, 2 or 3 amino acid substitutions in total in the six CDR sequences, in particular 1 or 2, especially 1 amino acid substitution. In these embodiments, the anti-LYPD3 antibody retains the antigen specificity of the antibody without said amino acid substitutions.

In further embodiments, the antibody according to the invention comprises a heavy chain variable region and a light chain variable region selected from the group consisting of
(ix) a heavy chain variable region having an amino acid sequence which is at least 90% identical to the amino acid sequence of SEQ ID NO: 71 over its entire length and comprising CDR-H1 having the amino acid sequence of SEQ ID NO: 65, CDR-H2 having the amino acid sequence of SEQ ID NO: 66 and CDR-H3 having the amino acid sequence of SEQ ID NO: 67, and
   a light chain variable region having an amino acid sequence which is at least 90% identical to the amino acid sequence of SEQ ID NO: 72 over its entire length and comprising CDR-L1 having the amino acid sequence of SEQ ID NO: 68, CDR-L2 having the amino acid sequence of SEQ ID NO: 69 and CDR-L3 having the amino acid sequence of SEQ ID NO: 70;
(x) a heavy chain variable region having an amino acid sequence which is at least 90% identical to the amino acid sequence of SEQ ID NO: 79 over its entire length and comprising CDR-H1 having the amino acid sequence of SEQ ID NO: 73, CDR-H2 having the amino acid sequence of SEQ ID NO: 74 and CDR-H3 having the amino acid sequence of SEQ ID NO: 75, and
   a light chain variable region having an amino acid sequence which is at least 90% identical to the amino acid sequence of SEQ ID NO: 80 over its entire length and comprising CDR-L1 having the amino acid sequence of SEQ ID NO: 76, CDR-L2 having the amino acid sequence of SEQ ID NO: 77 and CDR-L3 having the amino acid sequence of SEQ ID NO: 78;
(xi) a heavy chain variable region having an amino acid sequence which is at least 90% identical to the amino acid sequence of SEQ ID NO: 87 over its entire length and comprising CDR-H1 having the amino acid sequence of SEQ ID NO: 81, CDR-H2 having the amino acid sequence of SEQ ID NO: 82 and CDR-H3 having the amino acid sequence of SEQ ID NO: 83, and
   a light chain variable region having an amino acid sequence which is at least 90% identical to the amino acid sequence of SEQ ID NO: 88 over its entire length and comprising CDR-L1 having the amino acid sequence of SEQ ID NO: 84, CDR-L2 having the amino acid sequence of SEQ ID NO: 85 and CDR-L3 having the amino acid sequence of SEQ ID NO: 86;
(xii) a heavy chain variable region having an amino acid sequence which is at least 90% identical to the amino acid sequence of SEQ ID NO: 95 over its entire length and comprising CDR-H1 having the amino acid sequence of SEQ ID NO: 89, CDR-H2 having the amino acid sequence of SEQ ID NO: 90 and CDR-H3 having the amino acid sequence of SEQ ID NO: 91, and
   a light chain variable region having an amino acid sequence which is at least 90% identical to the amino acid sequence of SEQ ID NO: 96 over its entire length and comprising CDR-L1 having the amino acid sequence of SEQ ID NO: 92, CDR-L2 having the amino acid sequence of SEQ ID NO: 93 and CDR-L3 having the amino acid sequence of SEQ ID NO: 94;
(xiii) a heavy chain variable region having an amino acid sequence which is at least 90% identical to the amino acid sequence of SEQ ID NO: 103 over its entire length and comprising CDR-H1 having the amino acid sequence of SEQ ID NO: 97, CDR-H2 having the amino acid sequence of SEQ ID NO: 98 and CDR-H3 having the amino acid sequence of SEQ ID NO: 99, and
   a light chain variable region having an amino acid sequence which is at least 90% identical to the amino acid sequence of SEQ ID NO: 104 over its entire length and comprising the CDR-L1 having the amino acid sequence of SEQ ID NO: 100, CDR-L2 having the amino acid sequence of SEQ ID NO: 101 and CDR-L3 having the amino acid sequence of SEQ ID NO: 102;
(xiv) a heavy chain variable region having an amino acid sequence which is at least 90% identical to the amino acid sequence of SEQ ID NO: 111 over its entire length and comprising CDR-H1 having the amino acid sequence of SEQ ID NO: 105, CDR-H2 having the amino acid sequence of SEQ ID NO: 106 and CDR-H3 having the amino acid sequence of SEQ ID NO: 107, and
   a light chain variable region having an amino acid sequence which is at least 90% identical to the amino acid sequence of SEQ ID NO: 112 over its entire length and comprising CDR-L1 having the amino acid sequence of SEQ ID NO: 108, CDR-L2 having the amino acid sequence of SEQ ID NO: 109 and CDR-L3 having the amino acid sequence of SEQ ID NO: 110;
(xv) a heavy chain variable region having an amino acid sequence which is at least 90% identical to the amino acid sequence of SEQ ID NO: 119 over its entire length and comprising CDR-H1 having the amino acid sequence of SEQ ID NO: 113, CDR-H2 having the amino acid sequence of SEQ ID NO: 114 and CDR-H3 having the amino acid sequence of SEQ ID NO: 115, and
   a light chain variable region having an amino acid sequence which is at least 90% identical to the amino acid sequence of SEQ ID NO: 120 over its entire length and comprising CDR-L1 having the amino acid sequence of SEQ ID NO: 116, CDR-L2 having the amino acid sequence of SEQ ID NO: 117 and CDR-L3 having the amino acid sequence of SEQ ID NO: 118;
(xvi) a heavy chain variable region having an amino acid sequence which is at least 90% identical to the amino acid sequence of SEQ ID NO: 127 over its entire length and comprising CDR-H1 having the amino acid sequence of SEQ ID NO: 121, CDR-H2 having the amino acid sequence of SEQ ID NO: 122 and CDR-H3 having the amino acid sequence of SEQ ID NO: 123, and
   a light chain variable region having an amino acid sequence which is at least 90% identical to the amino acid sequence of SEQ ID NO: 128 over its entire length and comprising CDR-L1 having the amino acid sequence of SEQ ID NO: 124, CDR-L2 having the amino acid sequence of SEQ ID NO: 125 and CDR-L3 having the amino acid sequence of SEQ ID NO: 126; and
(xvii) a heavy chain variable region having an amino acid sequence which is at least 90% identical to the amino acid sequence of SEQ ID NO: 135 over its entire length and comprising CDR-H1 having the amino acid sequence of SEQ ID NO: 129, CDR-H2 having the amino acid sequence of SEQ ID NO: 130 and CDR-H3 having the amino acid sequence of SEQ ID NO: 131, and
   a light chain variable region having an amino acid sequence which is at least 90% identical to the amino acid sequence of SEQ ID NO: 136 over its entire length and comprising CDR-L1 having the amino acid sequence of SEQ ID NO: 132, CDR-L2 having the amino acid sequence of SEQ ID NO: 133 and CDR-L3 having the amino acid sequence of SEQ ID NO: 134.

In the above embodiments the sequence identity may in particular be at least 95%. In certain embodiments, the anti-LYPD3 antibody may additionally have 1, 2 or 3 amino acid substitutions in total in the six CDR sequences, in particular 1 or 2, especially 1 amino acid substitution. In these embodiments, the anti-LYPD3 antibody retains the antigen specificity of the antibody without said amino acid substitutions.

In particular, the antibody according to the invention comprises a heavy chain variable region and a light chain variable region selected from the group consisting of
(i) a heavy chain variable region comprising the amino acid sequence of SEQ ID NO: 7, and a light chain variable region comprising the amino acid sequence of SEQ ID NO: 8;
(ii) a heavy chain variable region comprising the amino acid sequence of SEQ ID NO: 15, and a light chain variable region comprising the amino acid sequence of SEQ ID NO: 16;
(iii) a heavy chain variable region comprising the amino acid sequence of SEQ ID NO: 23, and a light chain variable region comprising the amino acid sequence of SEQ ID NO: 24;
(iv) a heavy chain variable region comprising the amino acid sequence of SEQ ID NO: 31, and a light chain variable region comprising the amino acid sequence of SEQ ID NO: 32;
(v) a heavy chain variable region comprising the amino acid sequence of SEQ ID NO: 39, and a light chain variable region comprising the amino acid sequence of SEQ ID NO: 40;
(vi) a heavy chain variable region comprising the amino acid sequence of SEQ ID NO: 47, and a light chain variable region comprising the amino acid sequence of SEQ ID NO: 48;
(vii) a heavy chain variable region comprising the amino acid sequence of SEQ ID NO: 55, and a light chain variable region comprising the amino acid sequence of SEQ ID NO: 56; and
(viii) a heavy chain variable region comprising the amino acid sequence of SEQ ID NO: 63, and a light chain variable region comprising the amino acid sequence of SEQ ID NO: 64.

In preferred embodiments, the anti-LYPD3 antibody comprises a heavy chain variable region and a light chain variable region according to item (ii), above. In preferred embodiments, the anti-LYPD3 antibody comprises a heavy chain variable region and a light chain variable region according to item (v), above. In certain embodiments, the antibody is a humanized version of any one of the antibodies according to items (i) to (viii), above. The humanized version in particular has an amino acid sequence identity with the respective above antibody of at least 60%, especially at least 70%, preferably at least 80% and more preferably at least 90% over the entire length of the original sequence. In certain embodiments, the antibody according to the present invention is a humanized antibody derived from an antibody having a heavy chain variable region comprising the amino acid sequence of SEQ ID NO: 15, and a light chain variable region comprising the amino acid sequence of SEQ ID NO: 16, or derived from an antibody having a heavy chain variable region comprising the amino acid sequence of SEQ ID NO: 39, and a light chain variable region comprising the amino acid sequence of SEQ ID NO: 40.

In particular, the antibody according to the present invention is a humanized antibody comprising a heavy chain variable region and a light chain variable region selected from the group of
(i) a heavy chain variable region comprising the amino acid sequence of SEQ ID NO: 139, and a light chain variable region comprising the amino acid sequence of SEQ ID NO: 144;
(ii) a heavy chain variable region comprising the amino acid sequence of SEQ ID NO: 140, and a light chain variable region comprising the amino acid sequence of SEQ ID NO: 144;
(iii) a heavy chain variable region comprising the amino acid sequence of SEQ ID NO: 141, and a light chain variable region comprising the amino acid sequence of SEQ ID NO: 144;
(iv) a heavy chain variable region comprising the amino acid sequence of SEQ ID NO: 142, and a light chain variable region comprising the amino acid sequence of SEQ ID NO: 144;
(v) a heavy chain variable region comprising the amino acid sequence of SEQ ID NO: 142, and a light chain variable region comprising the amino acid sequence of SEQ ID NO: 143;
(vi) a heavy chain variable region comprising the amino acid sequence of SEQ ID NO: 142, and a light chain variable region comprising the amino acid sequence of SEQ ID NO: 145;
(vii) a heavy chain variable region comprising the amino acid sequence of SEQ ID NO: 142, and a light chain variable region comprising the amino acid sequence of SEQ ID NO: 146;
(viii) a heavy chain variable region comprising the amino acid sequence of SEQ ID NO: 138, and a light chain variable region comprising the amino acid sequence of SEQ ID NO: 145; and
(ix) a heavy chain variable region comprising the amino acid sequence of SEQ ID NO: 140, and a light chain variable region comprising the amino acid sequence of SEQ ID NO: 145.

The antibody according to the invention may also comprise a heavy chain variable region and a light chain variable region selected from the group consisting of
(ix) a heavy chain variable region comprising the amino acid sequence of SEQ ID NO: 71, and a light chain variable region comprising the amino acid sequence of SEQ ID NO: 72;
(x) a heavy chain variable region comprising the amino acid sequence of SEQ ID NO: 79, and a light chain variable region comprising the amino acid sequence of SEQ ID NO: 80;
(xi) a heavy chain variable region comprising the amino acid sequence of SEQ ID NO: 87, and a light chain variable region comprising the amino acid sequence of SEQ ID NO: 88;
(xii) a heavy chain variable region comprising the amino acid sequence of SEQ ID NO: 95, and a light chain variable region comprising the amino acid sequence of SEQ ID NO: 96;
(xiii) a heavy chain variable region comprising the amino acid sequence of SEQ ID NO: 103, and a light chain variable region comprising the amino acid sequence of SEQ ID NO: 104;
(xiv) a heavy chain variable region comprising the amino acid sequence of SEQ ID NO: 111, and a light chain variable region comprising the amino acid sequence of SEQ ID NO: 112;
(xv) a heavy chain variable region comprising the amino acid sequence of SEQ ID NO: 119, and a light chain variable region comprising the amino acid sequence of SEQ ID NO: 120;
(xvi) a heavy chain variable region comprising the amino acid sequence of SEQ ID NO: 127, and a light chain variable region comprising the amino acid sequence of SEQ ID NO: 128; and
(xvii) a heavy chain variable region comprising the amino acid sequence of SEQ ID NO: 135, and a light chain variable region comprising the amino acid sequence of SEQ ID NO: 136.

In embodiments wherein the heavy and/or light chain variable region comprises an amino acid sequence which has a certain identity to an amino acid sequence of SEQ ID NOS: 7, 8, 15, 16, 23, 24, 31, 32, 39, 40, 47, 48, 55, 56, 63, 64, 71, 72, 79, 80, 87, 88, 95, 96, 103, 104, 111, 112, 119, 120, 127, 128, 135 or 136, any sequence deviations to said amino acid sequence are in particular located in the framework regions, but not in the CDRs. Hence, in these embodiments the heavy and light chain variable regions comprise the respective CDR sequences as defined herein.

In specific embodiments, the antibody is capable of binding to human LYPD3 glycosylated with any one or more of GalNAcα1-, sialylated GalNAcα1-, Galβ1-3GalNAcα1-, and sialylated Galβ1-3GalNAcα1-, and comprises a heavy chain variable region and a light chain variable region selected from the group consisting of
(i) a heavy chain variable region comprising the complementarity determining regions (CDRs) CDR-H1 having the amino acid sequence of SEQ ID NO: 1, CDR-H2 having the amino acid sequence of SEQ ID NO: 2 and CDR-H3 having the amino acid sequence of SEQ ID NO: 3, and
   a light chain variable region comprising the complementarity-determining regions (CDRs) CDR-L1 having the amino acid sequence of SEQ ID NO: 4, CDR-L2 having the amino acid sequence of SEQ ID NO: 5 and CDR-L3 having the amino acid sequence of SEQ ID NO: 6;
(ii) a heavy chain variable region comprising the complementarity-determining regions (CDRs) CDR-H1 having the amino acid sequence of SEQ ID NO: 49, CDR-H2 having the amino acid sequence of SEQ ID NO: 50 and CDR-H3 having the amino acid sequence of SEQ ID NO: 51, and
   a light chain variable region comprising the complementarity-determining regions (CDRs) CDR-L1 having the amino acid sequence of SEQ ID NO: 52, CDR-L2 having the amino acid sequence of SEQ ID NO: 53 and CDR-L3 having the amino acid sequence of SEQ ID NO: 54;
(iii) a heavy chain variable region having an amino acid sequence which is at least 60% identical to the amino acid sequence of SEQ ID NO: 7 over its entire length and comprising the complementarity-determining regions (CDRs) CDR-H1 having the amino acid sequence of SEQ ID NO: 1, CDR-H2 having the amino acid sequence of SEQ ID NO: 2 and CDR-H3 having the amino acid sequence of SEQ ID NO: 3, and
   a light chain variable region having an amino acid sequence which is at least 60% identical to the amino acid sequence of SEQ ID NO: 8 over its entire length and comprising the complementarity-determining regions (CDRs) CDR-L1 having the amino acid sequence of SEQ ID NO: 4, CDR-L2 having the amino acid sequence of SEQ ID NO: 5 and CDR-L3 having the amino acid sequence of SEQ ID NO: 6;
(iv) a heavy chain variable region having an amino acid sequence which is at least 60% identical to the amino acid sequence of SEQ ID NO: 55 over its entire length and comprising CDR-H1 having the amino acid sequence of SEQ ID NO: 49, CDR-H2 having the amino acid sequence of SEQ ID NO: 50 and CDR-H3 having the amino acid sequence of SEQ ID NO: 51, and
   a light chain variable region having an amino acid sequence which is at least 60% identical to the amino acid sequence of SEQ ID NO: 56 over its entire length and comprising CDR-L1 having the amino acid sequence of SEQ ID NO: 52, CDR-L2 having the amino acid sequence of SEQ ID NO: 53 and CDR-L3 having the amino acid sequence of SEQ ID NO: 54;
(v) a heavy chain variable region comprising the amino acid sequence of SEQ ID NO: 7, and a light chain variable region comprising the amino acid sequence of SEQ ID NO: 8; and
(vi) a heavy chain variable region comprising the amino acid sequence of SEQ ID NO: 55, and a light chain variable region comprising the amino acid sequence of SEQ ID NO: 56.

In specific embodiments, the antibody is capable of binding to human LYPD3 glycosylated with any one or both of GalNAcα1- and Galβ1-3GalNAcα1-, and comprises a heavy chain variable region and a light chain variable region selected from the group consisting of
(i) a heavy chain variable region comprising the complementarity-determining regions (CDRs) CDR-H1 having the amino acid sequence of SEQ ID NO: 33, CDR-H2 having the amino acid sequence of SEQ ID NO: 34 and CDR-H3 having the amino acid sequence of SEQ ID NO: 35, and
   a light chain variable region comprising the complementarity-determining regions (CDRs) CDR-L1 having the amino acid sequence of SEQ ID NO: 36, CDR-L2 having the amino acid sequence of SEQ ID NO: 37 and CDR-L3 having the amino acid sequence of SEQ ID NO: 38;
(ii) a heavy chain variable region comprising the complementarity-determining regions (CDRs) CDR-H1 having the amino acid sequence of SEQ ID NO: 41, CDR-H2 having the amino acid sequence of SEQ ID NO: 42 and CDR-H3 having the amino acid sequence of SEQ ID NO: 43, and
   a light chain variable region comprising the complementarity-determining regions (CDRs) CDR-L1 having the amino acid sequence of SEQ ID NO: 44, CDR-L2 having the amino acid sequence of SEQ ID NO: 45 and CDR-L3 having the amino acid sequence of SEQ ID NO: 46;
(iii) a heavy chain variable region comprising the complementarity-determining regions (CDRs) CDR-H1 having the amino acid sequence of SEQ ID NO: 57, CDR-H2 having the amino acid sequence of SEQ ID NO: 58 and CDR-H3 having the amino acid sequence of SEQ ID NO: 59, and
   a light chain variable region comprising the complementarity-determining regions (CDRs) CDR-L1 having the amino acid sequence of SEQ ID NO: 60, CDR-L2 having the amino acid sequence of SEQ ID NO: 61 and CDR-L3 having the amino acid sequence of SEQ ID NO: 62;
(iv) a heavy chain variable region having an amino acid sequence which is at least 60% identical to the amino acid sequence of SEQ ID NO: 39 over its entire length and comprising CDR-H1 having the amino acid sequence of SEQ ID NO: 33, CDR-H2 having the amino acid sequence of SEQ ID NO: 34 and CDR-H3 having the amino acid sequence of SEQ ID NO: 35, and
   a light chain variable region having an amino acid sequence which is at least 60% identical to the amino acid sequence of SEQ ID NO: 40 over its entire length and comprising CDR-L1 having the amino acid sequence of SEQ ID NO: 36, CDR-L2 having the amino acid sequence of SEQ ID NO: 37 and CDR-L3 having the amino acid sequence of SEQ ID NO: 38;
(v) a heavy chain variable region having an amino acid sequence which is at least 60% identical to the amino acid sequence of SEQ ID NO: 47 over its entire length and comprising CDR-H1 having the amino acid sequence of SEQ ID NO: 41, CDR-H2 having the amino acid sequence of SEQ ID NO: 42 and CDR-H3 having the amino acid sequence of SEQ ID NO: 43, and
   a light chain variable region having an amino acid sequence which is at least 60% identical to the amino acid sequence of SEQ ID NO: 48 over its entire length and comprising CDR-L1 having the amino acid sequence of SEQ ID NO: 44, CDR-L2 having the amino acid sequence of SEQ ID NO: 45 and CDR-L3 having the amino acid sequence of SEQ ID NO: 46;
(vi) a heavy chain variable region having an amino acid sequence which is at least 60% identical to the amino acid sequence of SEQ ID NO: 63 over its entire length and comprising CDR-H1 having the amino acid sequence of SEQ ID NO: 57, CDR-H2 having the amino acid sequence of SEQ ID NO: 58 and CDR-H3 having the amino acid sequence of SEQ ID NO: 59, and
   a light chain variable region having an amino acid sequence which is at least 60% identical to the amino acid sequence of SEQ ID NO: 64 over its entire length and comprising the CDR-L1 having the amino acid sequence of SEQ ID NO: 60, CDR-L2 having the amino acid sequence of SEQ ID NO: 61 and CDR-L3 having the amino acid sequence of SEQ ID NO: 62;
(vii) a heavy chain variable region comprising the amino acid sequence of SEQ ID NO: 39, and a light chain variable region comprising the amino acid sequence of SEQ ID NO: 40;
(viii) a heavy chain variable region comprising the amino acid sequence of SEQ ID NO: 139, and a light chain variable region comprising the amino acid sequence of SEQ ID NO: 144;
(ix) a heavy chain variable region comprising the amino acid sequence of SEQ ID NO: 140, and a light chain variable region comprising the amino acid sequence of SEQ ID NO: 144;
(x) a heavy chain variable region comprising the amino acid sequence of SEQ ID NO: 141, and a light chain variable region comprising the amino acid sequence of SEQ ID NO: 144;
(xi) a heavy chain variable region comprising the amino acid sequence of SEQ ID NO: 142, and a light chain variable region comprising the amino acid sequence of SEQ ID NO: 144;
(xi) a heavy chain variable region comprising the amino acid sequence of SEQ ID NO: 142, and a light chain variable region comprising the amino acid sequence of SEQ ID NO: 143;
(xii) a heavy chain variable region comprising the amino acid sequence of SEQ ID NO: 142, and a light chain variable region comprising the amino acid sequence of SEQ ID NO: 145;
(xiii) a heavy chain variable region comprising the amino acid sequence of SEQ ID NO: 142, and a light chain variable region comprising the amino acid sequence of SEQ ID NO: 146;
(xiv) a heavy chain variable region comprising the amino acid sequence of SEQ ID NO: 138, and a light chain variable region comprising the amino acid sequence of SEQ ID NO: 145;
(xv) a heavy chain variable region comprising the amino acid sequence of SEQ ID NO: 140, and a light chain variable region comprising the amino acid sequence of SEQ ID NO: 145;
(xvi) a heavy chain variable region comprising the amino acid sequence of SEQ ID NO: 47, and a light chain variable region comprising the amino acid sequence of SEQ ID NO: 48; and
(xvii) a heavy chain variable region comprising the amino acid sequence of SEQ ID NO: 63, and a light chain variable region comprising the amino acid sequence of SEQ ID NO: 64.

In preferred embodiments, the antibody is capable of binding to human LYPD3 glycosylated with any one or both of GalNAcα1- and Galβ1-3GalNAcα1-, and comprises a heavy chain variable region and a light chain variable region according to any one of items (i), (iv), (vii) and (viii) to (xv), above.

In specific embodiments, the antibody is capable of binding to human LYPD3 glycosylated with any one or both of Galβ1-3GalNAcα1- and sialylated Galβ1-3GalNAcα1-, and comprises a heavy chain variable region and a light chain variable region selected from the group consisting of
(i) a heavy chain variable region comprising the complementarity-determining regions (CDRs) CDR-H1 having the amino acid sequence of SEQ ID NO: 25, CDR-H2 having the amino acid sequence of SEQ ID NO: 26 and CDR-H3 having the amino acid sequence of SEQ ID NO: 27, and
   a light chain variable region comprising the complementarity-determining regions (CDRs) CDR-L1 having the amino acid sequence of SEQ ID NO: 28, CDR-L2 having the amino acid sequence of SEQ ID NO: 29 and CDR-L3 having the amino acid sequence of SEQ ID NO: 30;
(ii) a heavy chain variable region having an amino acid sequence which is at least 60% identical to the amino acid sequence of SEQ ID NO: 31 over its entire length and comprising CDR-H1 having the amino acid sequence of SEQ ID NO: 25, CDR-H2 having the amino acid sequence of SEQ ID NO: 26 and CDR-H3 having the amino acid sequence of SEQ ID NO: 27, and
   a light chain variable region having an amino acid sequence which is at least 60% identical to the amino acid sequence of SEQ ID NO: 32 over its entire length and comprising CDR-L1 having the amino acid sequence of SEQ ID NO: 28, CDR-L2 having the amino acid sequence of SEQ ID NO: 3295 and CDR-L3 having the amino acid sequence of SEQ ID NO: 30; and
(iii) a heavy chain variable region comprising the amino acid sequence of SEQ ID NO: 31, and a light chain variable region comprising the amino acid sequence of SEQ ID NO: 32.

In specific embodiments, the antibody is capable of binding to human LYPD3 glycosylated with Galβ1-3GalNAcα1-, and comprises a heavy chain variable region and a light chain variable region selected from the group consisting of
(i) a heavy chain variable region comprising the complementarity-determining regions (CDRs) CDR-H1 having the amino acid sequence of SEQ ID NO: 9, CDR-H2 having the amino acid sequence of SEQ ID NO: 10 and CDR-H3 having the amino acid sequence of SEQ ID NO: 11, and
   a light chain variable region comprising the complementarity-determining regions (CDRs) CDR-L1 having the amino acid sequence of SEQ ID NO: 12, CDR-L2 having the amino acid sequence of SEQ ID NO: 13 and CDR-L3 having the amino acid sequence of SEQ ID NO: 14;
(ii) a heavy chain variable region comprising the complementarity-determining regions (CDRs) CDR-H1 having the amino acid sequence of SEQ ID NO: 17, CDR-H2 having the amino acid sequence of SEQ ID NO: 18 and CDR-H3 having the amino acid sequence of SEQ ID NO: 19, and
   a light chain variable region comprising the complementarity-determining regions (CDRs) CDR-L1 having the amino acid sequence of SEQ ID NO: 20, CDR-L2 having the amino acid sequence of SEQ ID NO: 21 and CDR-L3 having the amino acid sequence of SEQ ID NO: 22;
(iii) a heavy chain variable region having an amino acid sequence which is at least 60% identical to the amino acid sequence of SEQ ID NO: 15 over its entire length and comprising CDR-H1 having the amino acid sequence of SEQ ID NO: 9, CDR-H2 having the amino acid sequence of SEQ ID NO: 10 and CDR-H3 having the amino acid sequence of SEQ ID NO: 11, and
   a light chain variable region having an amino acid sequence which is at least 60% identical to the amino acid sequence of SEQ ID NO: 16 over its entire length and comprising CDR-L1 having the amino acid sequence of SEQ ID NO: 12, CDR-L2 having the amino acid sequence of SEQ ID NO: 13 and CDR-L3 having the amino acid sequence of SEQ ID NO: 14;
(iv) a heavy chain variable region having an amino acid sequence which is at least 60% identical to the amino acid sequence of SEQ ID NO: 23 over its entire length and comprising CDR-H1 having the amino acid sequence of SEQ ID NO: 17, CDR-H2 having the amino acid sequence of SEQ ID NO: 18 and CDR-H3 having the amino acid sequence of SEQ ID NO: 19, and
   a light chain variable region having an amino acid sequence which is at least 60% identical to the amino acid sequence of SEQ ID NO: 24 over its entire length and comprising CDR-L1 having the amino acid sequence of SEQ ID NO: 20, CDR-L2 having the amino acid sequence of SEQ ID NO: 21 and CDR-L3 having the amino acid sequence of SEQ ID NO: 22;
(v) a heavy chain variable region comprising the amino acid sequence of SEQ ID NO: 15, and a light chain variable region comprising the amino acid sequence of SEQ ID NO: 16; and
(vi) a heavy chain variable region comprising the amino acid sequence of SEQ ID NO: 23, and a light chain variable region comprising the amino acid sequence of SEQ ID NO: 24.

In preferred embodiments, the antibody is capable of binding to human LYPD3 glycosylated with Galβ1-3GalNAcα1-, and comprises a heavy chain variable region and a light chain variable region according to any one of items (i), (iii) and (v), above.

In the above embodiments the sequence identity may in particular be at least 70%, preferably at least 80%, and more preferably at least 90%. In the above embodiments which refer to a sequence identity, the antibody in particular is a humanized version of the respective antibody wherein the changes in the amino acid sequence are substitutions to amino acid residues of a related human antibody sequence.

In certain embodiments, the anti-LYPD3 antibody may additionally have 1, 2 or 3 amino acid substitutions in total in the six CDR sequences, in particular 1 or 2, especially 1 amino acid substitution. In these embodiments, the anti-LYPD3 antibody retains the antigen specificity of the antibody without said amino acid substitutions.

In certain embodiments, the antibody comprises an Fc region. The antibody may especially be a whole antibody. In particular, the antibody may comprise two heavy chains and two light chains. The antibody may be of any isotype, and in particular is an IgG-type antibody, especially IgG1, IgG2 or IgG4. In specific embodiments, the antibody is an IgG1-type antibody. The antibody in particular is capable of binding to one or more human Fc receptors, especially human Fcγ receptors such as Fcγ receptor IIIa. In certain embodiments, the anti-LYPD3 antibody is a chimeric, humanized or human antibody.

In further embodiments, the anti-LYPD3 antibody is a fragment of an antibody. Especially the fragment is selected from the group consisting of (i) Fab fragments; (ii) F(ab)₂ fragments; (iii) Fd fragments; (iv) Fv fragments; (v) scFv fragments; and (vi) (Fv)₂ fragments. In certain embodiments, the anti-LYPD3 antibody does not comprise an Fc region.

In certain embodiment, the anti-LYPD3 antibody is glycosylated, especially N-glycosylated. In particular, the antibody has a glycosylation site in the second constant domain of the heavy chain (CH2). An antibody normally has two heavy chains having identical amino acid sequences. Hence, the antibody preferably has at least two glycosylation sites, one in each of its two CH2 domains. This glycosylation site in particular is at an amino acid position corresponding to amino acid position 297 of the heavy chain according to the Kabat numbering and has the amino acid sequence motive Asn Xaa Ser/Thr wherein Xaa may be any amino acid except proline. The N-linked glycosylation at Asn297 is conserved in mammalian IgGs as well as in homologous regions of other antibody isotypes. Due to optional additional amino acids which may be present in the variable region or other sequence modifications, the actual position of this conserved glycosylation site may vary in the amino acid sequence of the antibody.

In preferred embodiments, the anti-LYPD3 antibody does not comprise N-glycolyl neuraminic acids (NeuGc) or detectable amounts of NeuGc. Furthermore, the antibody preferably also does not comprise Galili epitopes (Galα1,3-Gal structures) or detectable amounts of the Galili epitope. In particular, the relative amount of glycans carrying NeuGc and/or Galα1,3-Gal structures is less than 0.1% or even less than 0.02% of the total amount of glycans attached to the Fc part of the antibodies in the antibody population.

In other embodiments, the anti-LYPD3 antibody is not glycosylated at its CH2 domains. In these embodiments, the CH2 domain of the antibody may be mutated, for example by substituting the asparagine residue at position 297 of the heavy chain (or a corresponding position) by any other amino acid, for example alanine or glutamine. Antibodies lacking glycosylation at the CH2 domain have reduced binding to Fcγ receptors and thus, reduced effector functions. In further embodiments, the anti-LYPD3 antibody may have other or additional amino acid substitutions which reduce Fc receptor binding, including, for example, Leu235Glu (“LE mutation”), Leu234Ala/Leu235Ala (“LALA” mutation), Ser228Pro/Leu235Glu (“SPLE” mutation), Leu234Ala/Leu235Ala/Pro329Gly (“LALA-PG” mutation) and combinations thereof.

The present invention further provides an anti-LYPD3 antibody which competes for binding to LYPD3 with an antibody as described herein, especially with an antibody according to the second aspect of the invention. In certain embodiments, the competitive anti-LYPD3 antibody competes with an antibody comprising a heavy chain variable region and a light chain variable region selected from the group consisting of
(i) a heavy chain variable region comprising the amino acid sequence of SEQ ID NO: 7, and a light chain variable region comprising the amino acid sequence of SEQ ID NO: 8;
(ii) a heavy chain variable region comprising the amino acid sequence of SEQ ID NO: 15, and a light chain variable region comprising the amino acid sequence of SEQ ID NO: 16;
(iii) a heavy chain variable region comprising the amino acid sequence of SEQ ID NO: 23, and a light chain variable region comprising the amino acid sequence of SEQ ID NO: 24;
(iv) a heavy chain variable region comprising the amino acid sequence of SEQ ID NO: 31, and a light chain variable region comprising the amino acid sequence of SEQ ID NO: 32;
(v) a heavy chain variable region comprising the amino acid sequence of SEQ ID NO: 39, and a light chain variable region comprising the amino acid sequence of SEQ ID NO: 40;
(vi) a heavy chain variable region comprising the amino acid sequence of SEQ ID NO: 47, and a light chain variable region comprising the amino acid sequence of SEQ ID NO: 48;
(vii) a heavy chain variable region comprising the amino acid sequence of SEQ ID NO: 55, and a light chain variable region comprising the amino acid sequence of SEQ ID NO: 56; and
(viii) a heavy chain variable region comprising the amino acid sequence of SEQ ID NO: 63, and a light chain variable region comprising the amino acid sequence of SEQ ID NO: 64.

Assays for determining competitive binding of two antibodies are well known in the art. For example, an ELISA can be used wherein LYPD3 is immobilized and the first antibodies is labeled and added with an excess of the second antibody to the immobilized LYPD3. If the label can be detected in the sample with the immobilized LYPD3 after washing, no competitive binding was observed. In preferred embodiments, control experiments wherein the second antibody is labeled and the first antibody is added in excess are performed. Alternatively, also one of the antibodies may be immobilized and LYPD3 may be labeled and added with an excess of the other antibody.

### 2. Production of the Anti-LYPD3 Antibodies

The anti-LYPD3 antibody is preferably recombinantly produced in a host cell. Hence, the antibody in particular is a monoclonal antibody. The host cell used for the production of the antibody may be any host cells which can be used for antibody production. Suitable host cells are in particular eukaryotic host cells, especially mammalian host cells. Exemplary host cells include yeast cells such as Pichia pastoris cell lines, insect cells such as SF9 and SF21 cell lines, plant cells, bird cells such as EB66 duck cell lines, rodent cells such as CHO, NS0, SP2/0 and YB2/0 cell lines, and human cells such as HEK293, PER.C6, CAP, CAP-T, AGE1.HN, Mutz-3 and KG1 cell lines.

In certain embodiments, the anti-LYPD3 antibody is produced recombinantly in a human cell line, in particular in a human myeloid leukemia cell line. Preferred human cell lines which can be used for production of the anti-LYPD3 antibody as well as suitable production procedures are described in WO 2008/028686 A2. In a specific embodiment, the anti-LYPD3 antibody is obtained by expression in a human myeloid leukemia cell line selected from the group consisting of NM-H9D8, NM-H9D8-E6 and NM-H9D8-E6Q12. These cell lines were deposited under the accession numbers DSM ACC2806 (NM-H9D8; deposited on Sep. 15, 2006), DSM ACC2807 (NM-H9D8-E6; deposited on Oct. 5, 2006) and DSM ACC2856 (NM-H9D8-E6Q12; deposited on Aug. 8, 2007) according to the requirements of the Budapest Treaty at the Deutsche Sammlung von Mikroorganismen und Zellkulturen (DSMZ), Inhoffenstraße 7B, 38124 Braunschweig (DE) by Glycotope GmbH, Robert-Rössle-Str. 10, 13125 Berlin (DE). NM-H9D8 cells provide a glycosylation pattern with a high degree of sialylation, a high degree of bisecting GlycNAc, a high degree of galactosylation and a high degree of fucosylation. NM-H9D8-E6 and NM-H9D8-E6Q12 cells provide a glycosylation pattern similar to that of NM-H9D8 cells, except that the degree of fucosylation is very low. Other suitable cell lines include K562, a human myeloid leukemia cell line present in the American Type Culture Collection (ATCC CCL-243), CHO cells, as well as cell lines derived from the aforementioned. In specific embodiments, the anti-LYPD3 antibody is produced recombinantly in CHO cells, especially in CHO dhfr⁻ cells.

### 3. Conjugates of the Anti-LYPD3 Antibodies

In specific embodiments, the anti-LYPD3 antibody is provided as conjugate comprising the antibody conjugated to a further agent such as a detectable marker or a therapeutically active substance. The antibody can be conjugated to one or more further agents. If more than one further agent is present in the conjugate, these further agents may be identical or different, and in particular are all identical. Conjugation of the further agent to the antibody can be achieved using any methods known in the art. The further agent may be covalently, in particular by fusion or chemical coupling, or non-covalently attached to the antibody. In certain embodiments, the further agent is covalently attached to the antibody, especially via a linker moiety. The linker moiety may be any chemical entity suitable for attaching the further agent to the antibody.

The further agent preferably is useful in therapy, diagnosis, prognosis and/or monitoring of a disease, in particular cancer. For example, the further agent may be selected from the group consisting of radionuclides, chemotherapeutic agents, antibodies, bispecific antibodies or antibody fragments, in particular those of different species and/or different specificity than the anti-LYPD3 antibody, enzymes, interaction domains, detectable labels, toxins, cytolytic components, immunomodulators, immunoeffectors, cytokines, chemokines, MHC class I or class Il antigens, and liposomes.

In certain embodiments, the further agent is a polypeptide or protein. This polypeptide or protein may in particular be fused to a polypeptide chain of the anti-LYPD3 antibody. In certain embodiments, the further agent being a polypeptide or protein is fused to the C terminus of an antibody light chain of the anti-LYPD3 antibody. In embodiments wherein the anti-LYPD3 antibody comprises two antibody light chains, a further agent being a polypeptide or protein may be fused to the C terminus of each of the two antibody light chains. In further embodiments, the further agent being a polypeptide or protein is fused to the C terminus of an antibody heavy chain of the anti-LYPD3 antibody. In embodiments wherein the antibody comprises two antibody heavy chains, a further agent being a polypeptide or protein may be fused to the C terminus of each of the two antibody heavy chains. The further agents may be identical or different and in particular have the same amino acid sequence. In embodiments wherein the antibody does not comprise one or more light chains and one or more heavy chains, for example in cases where the antibody is an antibody fragment, a further agent being a polypeptide or protein may be fused to the C terminus or the N terminus of a polypeptide chain of the antibody. Suitable examples of such further agents being a polypeptide or protein may be selected from the group consisting of cytokines, chemokines, antibodies, antigen binding fragments, enzymes, and interaction domains.

In certain embodiments, the further agent being a polypeptide or protein is a checkpoint antibody which blocks and/or triggers activating signals. Examples of respective targets include CD40, CD3, CD137 (4-1BB), OX40, GITR, CD27, CD278 (ICOS), CD154 (CD40 ligand), CD270 (HVEM) and CD258 (LIGHT) as activating targets, CTLA4, PD1, CD80, CD244, A2AR, B7-H3 (CD276), B7-H4 (VTCN1), BTLA, IDO, KIR, LAG3, TIM-3, VISTA and phosphatidylserine as inhibitory targets, and their respective ligands such as PDL1. In further embodiment the further agent being a polypeptide or protein is an anti-cancer antibody directed to a tumor-associated antigen. Exemplary suitable tumor targets and anti-cancer antibodies which may be used as fusion partner are described below with respect to combination therapies.

In further embodiments, the further agent being a polypeptide or protein is an immunomodulatory compound such as a chemokine, cytokine or growth factor. Suitable cytokines in this respect include interferons such as interferon-α, interferon-β and interferon-γ, and interleukins such as IL-15. Suitable growth factors include G-CSF and GM-CSF.

The conjugate comprising the anti-LYPD3 antibody conjugated to a further agent in particular is a chimeric antigen receptor (CAR). Such conjugates are also referred herein as anti-LYPD3 CARs.

In these embodiments, the further agent is an antigen receptor, especially a T cell receptor or a T cell co-receptor, or a part and/or chimera thereof. In particular, the anti-LYPD3 antibody is fused to a transmembrane domain and an intracellular T-cell signaling domain, forming a chimeric antigen receptor (CAR). The intracellular domain is in particular derived from one or more T cell receptors or co-receptors. Optionally, the CAR further comprises a hinge region between the antibody and the transmembrane domain.

In these embodiments, the anti-LYPD3 antibody in particular is a single chain antibody fragment which comprises the heavy chain variable region and the light chain variable region in one polypeptide chain, especially a scFv fragment. The hinge region may for example be based on a hinge region or membrane-proximal region of a member of the immunoglobulin superfamily. Exemplary hinge regions include those derived from IgG, CD8 and CD28. The transmembrane domain may be a hydrophobic alpha helix that spans the cell membrane. It is for example derived from CD28. The intracellular T-cell signaling domain in particular comprises the cytoplasmic domain of the ζ chain of the T cell receptor. In addition, the intracellular T-cell signaling domain may comprise further domains of co-stimulatory proteins of T cells. Exemplary further domains include signaling domains from CD28, CD27, CD134 (OX40), and CD137 (4-1BB).

An exemplary CAR may comprise, from N terminus to C terminus, (i) the anti-LYPD3 antibody in the form of a scFv fragment, (ii) an extracellular hinge region derived from CD8, (iii) a transmembrane domain derived from CD28, (iv) a cytoplasmic signaling domain derived from CD28, and (v) a signaling domain derived from the T cell receptor ζ-chain.

Alternatively, the anti-LYPD3 antibody, especially in single chain format such as scFv, may be fused N terminally to a CD3 chain of the T cell receptor complex, especially the CD3ε chain, to form a chimeric antigen receptor. Or the anti-LYPD3 antibody, especially in single chain format such as scFv, may be fused to a binding domain which is capable of specifically binding to naturally occurring or engineered receptors on T cells or NK cells.

In certain embodiments, the further agent is a cytotoxic or chemotherapeutic agent, especially a cytotoxin. Specific examples of chemotherapeutic agents that can be conjugated as further agent include alkylating agents such as cisplatin, anti-metabolites, plant alkaloids and terpenoids, vinca alkaloids, podophyllotoxin, taxanes such as taxol, topoisomerase inhibitors such as irinotecan and topotecan, antineoplastics such as doxorubicin or microtubule inhibitors such as auristatins and maytansin/maytansinoids.

The chemotherapeutic agent may in particular be selected from a group consisting of a V-ATPase inhibitor, a pro-apoptotic agent, a Bcl2 inhibitor, an MCL1 inhibitor, a HSP90 inhibitor, an IAP inhibitor, an mTor inhibitor, a microtubule stabilizer, a microtubule destabilizer, an auristatin, a dolastatin, a maytansin, a maytansinoid, amatoxin, a methionine aminopeptidase, an inhibitor of nuclear export of proteins CRM1, a DPPIV inhibitor, proteasome inhibitors, inhibitors of phosphoryl transfer reactions in mitochondria, a protein synthesis inhibitor, a kinase inhibitor, a CDK2 inhibitor, a CDK9 inhibitor, a kinesin inhibitor, an HDAC inhibitor, a topoisomerase I inhibitor, a DNA damaging agent, a DNA alkylating agent, a DNA intercalator, a DNA minor groove binder, a DHFR inhibitor, an inhibitor of microtubule formation, a stabilizer of microtubuli, a stabilizer of actin, a topoisomerase II inhibitor, a platinum compound, a ribosome inhibitor, an RNA polymerase II inhibitor and a bacterial toxin. In specific embodiments, the chemotherapeutic agent attached to the anti-LYPD3 antibody is selected from the group consisting of an auristatin, a maytansinoid, a topoisomerase I inhibitor, a DNA damaging agent, a DNA alkylating agent and a DNA minor groove binder.

In some embodiments of the chemotherapeutic agent is a maytansin or maytansinoid. Specific examples of maytansinoids useful for conjugation include maytansinol, N^{2′}-deacetyl-N^{2′}-(3-mercapto-1-oxopropyl)-maytansine (DM1), N^{2′}-deacetyl-N^{2′}-(4-mercapto-1-oxopentyl)-maytansine (DM3), and N^{2′}-deacetyl-N^{2′}-(4-methyl-4-mercapto-1-oxopentyl)-maytansine (DM4). In particular, DM1 or DM4 is attached to the anti-LYPD3 antibody. In some embodiments, the chemotherapeutic agent attached to the anti-LYPD3 antibody is an auristatin, in particular monomethyl auristatin F (MMAF), monomethyl auristatin E (MMAE) or auristatin T. In some embodiments, the chemotherapeutic agent attached to the anti-LYPD3 antibody is a DNA minor groove binder, in particular pyrrolobenzodiazepine (PBD), pyrrolobenzodiazepine dimer (PBD dimer), duocarmycin, duocarmycin-hydroxybenzamide-azaindole (DUBA), seco-duocarmycin-hydroxybenzamide-azaindole (seco-DUBA) or doxorubicin. In some embodiments, the chemotherapeutic agent attached to the anti-LYPD3 antibody is a DNA alkylating agent, indolinobenzodiazepine or in particular oxazolidinobenzodiazepine. In some embodiments, the chemotherapeutic agent attached to the anti-LYPD3 antibody is a DNA damaging agent, in particular calicheamicin. In some embodiments, the chemotherapeutic agent attached to the anti-LYPD3 antibody is a topoisomerase I inhibitor, in particular camptothecin and its derivatives such as 7-ethyl-10-hydroxy-camptothecin (SN-38), (S)-9-dimethylaminomethyl-10-hydroxycamptothecin (topotecan), (1S,9S)-1-amino-9-ethyl-5-fluoro-1,2,3,9,12,15-hexahydro-9-hydroxy-4-methyl-10H,13H-benzo[de]pyrano [3′,4′:6,7]indolizino[1,2-b]quinoline-10,13-dione (Exatecan (DX-8951f)) and DXd. In some embodiments, the chemotherapeutic agent attached to the anti-LYPD3 antibody is an inhibitor of microtubule formation, in particular a tubulysin, an ansamitocin, podophyllotoxin or vinblastine. In some embodiments, the chemotherapeutic agent attached to the anti-LYPD3 antibody is a stabilizer of microtubuli, in particular paclitaxel or an epothilone. In some embodiments, the chemotherapeutic agent attached to the anti-LYPD3 antibody is a stabilizer of actin, in particular a phallotoxin. In some embodiments, the chemotherapeutic agent attached to the anti-LYPD3 antibody is a topoisomerase II inhibitor, in particular teniposide, XK469, razoxane, amsacrine, idarubicin or mebarone. In some embodiments, the chemotherapeutic agent attached to the anti-LYPD3 antibody is a platinum compound, in particular cisplatin, carboplatin, oxaliplatin, nedaplatin, triplatin tetranitrate, phenanthriplatin, picoplatin or sattraplatin. In some embodiments, the chemotherapeutic agent attached to the anti-LYPD3 antibody is a ribosome inhibitor, in particular ricin, saporin, abrin, diphtheria toxin or exotoxin A. In some embodiments, the chemotherapeutic agent attached to the anti-LYPD3 antibody is an RNA polymerase II inhibitor, in particular an amatoxin, such as, for example, amanitin. In some embodiments, the chemotherapeutic agent attached to the anti-LYPD3 antibody is a bacterial toxin, in particular anthrax toxin. Suitable antibody drug conjugates are also described in EP 16 151 774.3 and LU 92659, to which is explicitly referred to herewith.

Further suitable toxins which may be conjugated to the anti-LYPD3 antibody are described below with respect to combination therapies.

### 4. Nucleic Acids Encoding the Anti-LYPD3 Antibodies

In a further aspect, the present invention provides a nucleic acid encoding the anti-LYPD3 antibody. The nucleic acid sequence of said nucleic acid may have any nucleotide sequence suitable for encoding the antibody. However, preferably the nucleic acid sequence is at least partially adapted to the specific codon usage of the host cell or organism in which the nucleic acid is to be expressed, in particular the human codon usage. The nucleic acid may be double-stranded or single-stranded DNA or RNA, preferably double-stranded DNA such as cDNA or single-stranded RNA such as mRNA. It may be one consecutive nucleic acid molecule or it may be composed of several nucleic acid molecules, each coding for a different part of the antibody.

If the anti-LYPD3 antibody is composed of more than one different amino acid chain, such as a light chain and a heavy chain, the nucleic acid may, for example, be a single nucleic acid molecule containing several coding regions each coding for one of the amino acid chains of the antibody, preferably separated by regulatory elements such as IRES elements in order to generate separate amino acid chains, or the nucleic acid may be composed of several nucleic acid molecules wherein each nucleic acid molecule comprises one or more coding regions each coding for one of the amino acid chains of the antibody. Alternatively, the nucleic acid may be a single nucleic acid molecule containing one coding region which encodes for the heavy chain and the light chain, separated by a self-cleaving peptide such as a 2A peptide, and/or a linker peptide containing a protease recognition site such as a furin recognition site. In addition to the coding regions encoding the antibody, the nucleic acid may also comprise further nucleic acid sequences or other modifications which, for example, may code for other proteins, may influence the transcription and/or translation of the coding region(s), may influence the stability or other physical or chemical properties of the nucleic acid, or may have no function at all.

In certain embodiments, the nucleic acid is a viral vector which can be used for the infection of human cells. These viral vectors may be suitable, for example, for therapy of humans, e.g. by directing infection and or replication of the virus to disease cells such as tumor cells, or be modifying T cells in embodiments where the anti-LYPD3 antibody is in the form of a chimeric antigen receptor to obtain CAR T cells.

In a further aspect, the present invention provides an expression cassette or vector comprising a nucleic acid according to the invention and a promoter operatively connected with said nucleic acid. In addition, the expression cassette or vector may comprise further elements, in particular elements which are capable of influencing and/or regulating the transcription and/or translation of the nucleic acid, the amplification and/or reproduction of the expression cassette or vector, the integration of the expression cassette or vector into the genome of a host cell, and/or the copy number of the expression cassette or vector in a host cell. Suitable expression cassettes and vectors comprising respective expression cassettes for expressing antibodies are well known in the prior art and thus, need no further description here.

### 5. Host Cells

Furthermore, the present invention provides a host cell comprising the nucleic acid according to the invention or the expression cassette or vector according to the invention. The host cell may be any host cell. It may be an isolated cell or a cell comprised in a tissue. Preferably, the host cell is a cultured cell, in particular a primary cell or a cell of an established cell line, preferably a tumor-derived cell. Suitable host cells are in particular eukaryotic host cells, especially mammalian host cells. Exemplary host cells include yeast cells such as Pichia pastoris cell lines, insect cells such as SF9 and SF21 cell lines, plant cells, bird cells such as EB66 duck cell lines, rodent cells such as CHO, NS0, SP2/0 and YB2/0 cell lines, and human cells such as HEK293, PER.C6, CAP, CAP-T, AGE1.HN, Mutz-3 and KG1 cell lines.

In a preferred embodiment of the invention the host cell is a CHO cell or a cell derived from human myeloid leukaemia cells. Preferably, it is selected from the following cells or cell lines: K562, KG1, MUTZ-3, CHO or a cell or cell line derived therefrom. The host cell is preferably selected from the group consisting of CHO, NM-H9D8, NM-H9D8-E6, NM H9D8-E6Q12, and a cell or cell line derived from anyone of said host cells. These cell lines and their properties are described in detail in the PCT-application WO 2008/028686 A2. In certain embodiments, the host cell is optimized for expression of glycoproteins, in particular antibodies, having a specific glycosylation pattern. Preferably, the codon usage in the coding region of the nucleic acid according to the invention and/or the promoter and the further elements of the expression cassette or vector are compatible with and, more preferably, optimized for the type of host cell used. Preferably, the anti-LYPD3 antibody is produced by a host cell or cell line as described above.

The present invention further provides a host cell carrying an anti-LYPD3 CAR. Such a host cell is also called CAR cell herein. The CAR cell in particular comprises the nucleic acid according to the invention or the expression cassette or vector according to the invention, which encoding the anti-LYPD3 CAR. In certain embodiments, the CAR cell is engineered to express the anti-LYPD3 CAR, for example by introducing a vector comprising an expression cassette for the anti-LYPD3 CAR.

In specific embodiments, the CAR cell is a white blood cell, especially a lymphocyte such as a T cell, a NK cell and a NKT cell, or a monocyte such as a macrophage. The CAR cell in particular is a primary white blood cell. In certain embodiments, the CAR cell is selected from the group consisting of primary T cells, primary NK cells, primary NKT cells and primary macrophages, and especially is a primary T cell.

### 6. Pharmaceutical Compositions and Therapeutic Use

In another aspect, the present invention provides a composition comprising the anti-LYPD3 antibody, the nucleic acid, the expression cassette or vector, the host cell, or the conjugate. The composition may also contain more than one of these components. Furthermore, the composition may comprise one or more further components selected from the group consisting of solvents, diluents, and excipients. Preferably, the composition is a pharmaceutical composition. In this embodiment, the components of the composition preferably are all pharmaceutically acceptable. The composition may be a solid or fluid composition, in particular a—preferably aqueous—solution, emulsion or suspension or a lyophilized powder.

The anti-LYPD3 antibody or the conjugate thereof or the CAR cell in particular is useful in medicine, in particular in therapy, diagnosis, prognosis and/or monitoring of a disease, in particular a disease as described herein, for example cancer and infections, preferably cancer. Therefore, in a further aspect, the invention provides the anti-LYPD3 antibody, the nucleic acid, the expression cassette or vector, the host cell, the conjugate, or the composition for use in medicine. Preferably, the use in medicine is a use in the treatment, prognosis, diagnosis and/or monitoring of a disease, especially a disease associated with LYPD3. Exemplary diseases include diseases associated with abnormal cell growth such as cancer, adolescent idiopathic scoliosis, cholesteryl ester transfer protein (CETP) deficiency, fish-eye disease, combined hyperlipidemia, citrullinemia and familial hypercholesterolemia.

In a preferred embodiment, the disease is cancer. Preferably the cancer is selected from the group consisting of head and neck cancer, colon cancer, colorectal cancer, hepatocellular carcinoma, skin cancer, cervical cancer, breast cancer, ovarian cancer, prostate cancer, renal cancer, esophageal cancer, lung cancer and genital region cancer. The cancer may for example be head and neck squamous cell carcinoma, esophageal carcinoma, breast carcinoma, cervical carcinoma, skin carcinoma, or a squamous cell carcinoma in the anal area, rectum, oral cavity, lip, bucca cavioris, nose, penis, vulva, epiglottis, tongue or skin.

The cancer is LYPD3 positive and in particular comprises cancer cells which carry LYPD3 on their cell surface. In specific embodiments, the anti-LYPD3 antibody is used in combination with another anti-cancer therapeutic agent. Said further therapeutic agent may be any known anti-cancer drug and in particular may be an antibody against a cancer antigen. Suitable antibodies for combination with the anti-LYPD3 antibody include anti-EGFR antibodies such as cetuximab (Erbitux), tomuzotuximab, panitumomab (Vectibix) and nimotuzumab (Theraloc), anti-HER2 antibodies such as trastuzumab (Herceptin), timigutuzumab and pertuzumab; anti-VEGF antibodies such as bevacizumab (Avastin) and vanuzizumab; anti-CD52 antibodies such as alemtuzumab (Campath); anti-CD30 antibodies such as brentuximab (Adcetris); anti-CD33 antibodies such as gemtuzumab (Mylotarg); anti-CD20 antibodies such as rituximab (Rituxan, Mabthera), tositumomab (Bexxar) and ibritumomab (Zevalin); anti-CTLA-4 antibodies such as ipilimumab and tremelimumab, anti-PD1/PD-L1 antibodies such as pembrolizumab, nivolumab, cemiplimab, atezolizumab, durvalumab and avelumab, antibodies against TNF and TNFR superfamily members such as urelumab, MEDI6469, TRX518, and varilumab; CSF1R antibodies such as emactuzumab; anti-B7-H3 antibodies such as enoblituzumab; anti-LAG3 antibodies; anti-4-1BB antibodies; anti-ICOS antibodies; and anti-OX-40 antibodies. Further suitable antibodies for combination with the anti-LYPD3 antibody include bispecific antibodies, especially immune cell recruiting bispecific antibodies, such as blinatumomab (targeting CD19 and CD3), odronextamab (targeting CD20 and CD3), Regn4018 (targeting MUC16 ad CD3), Regn4336 (targeting PSMA and CD3) and Regn7075 (targeting EGFR and CD28).

Further anti-cancer therapeutic agents which may be combined with the anti-LYPD3antibody and optionally one or more further antibodies may be selected from the group consisting of taxanes such as paclitaxel (Taxol), docetaxel (Taxotere) and SB-T-1214; cyclophosphamide; lapatinib; erlotinib; imatinib; pazopanib; capecitabine; cytarabine; vinorelbine; gemcitabine; anthracyclines such as daunorubicin, doxorubicin, epirubicin, idarubicin, valrubicin and mitoxantrone; aromatase inhibitors such as aminoglutethimide, testolactone (Teslac), anastrozole (Arimidex), letrozole (Femara), exemestane (Aromasin), vorozole (Rivizor), formestane (Lentaron), fadrozole (Afema), 4-hydroxyandrostenedione, 1,4,6-androstatrien-3,17-dione (ATD) and 4-androstene-3,6,17-trione (6-OXO); topoisomerase inhibitors such as irinotecan, topotecan, camptothecin, lamellarin D, etoposide (VP-16), teniposide, doxorubicin, daunorubicin, mitoxantrone, amsacrine, ellipticines, aurintricarboxylic acid and HU-331; platinum based chemotherapeutic agents such as cis-diamminedichloroplatinum(II) (cisplatin), cis-diammine(1,1-cyclobutanedicarboxylato)platinum(II) (carboplatin) and [(1R,2R)-cyclohexane-1,2-diamine](ethanedioato-O,O′)platinum(II) (oxaliplatin); antimetabolites, in particular antifolates such as methotrexate, pemetrexed, raltitrexed and pralatrexate, pyrimidine analogues such as fluoruracil, gemcitabine, floxuridine, 5-fluorouracil and tegafur-uracil, and purine analogues; and inhibitors of the enzyme poly ADP ribose polymerase (PARP inhibitors) such as olaparib, rucaparib, niraparib and talazoparib. Further suitable toxins which may be used in combination with the anti-LYPD3 antibody are described above with respect to agents which may be conjugated to the antibody.

Treatment with the anti-LYPD3 antibody may further be combined with immunostimulatory agents, cytokines, chemokines, radiation therapy, therapeutic cells such as native immune cells or engineered immune cells (CAR-T cells, TCR-T cells, CAR-NK cells, CAR-monocytes, CAR-NKT cells, etc.), vaccines such as protein, peptide or RNA vaccines, B-Raf inhibitors such as vemurafenib, dexametasona, protease inhibitors such as bortezomib, and lenalidomide.

For use in the treatment of cancer wherein the cells express LYPD3, the antibody may be coupled to a further agent as described above, wherein the further agent preferably is a cytotoxic agent such as a radionuclide or a cytotoxin. Exemplary cytotoxic agents are described above. Cytotoxic agents also include precursor compounds which only develop cytotoxic activity upon activation, e.g. by irradiation with light or enzymatic reaction inside the body. One or more of the anti-cancer therapeutic agents described above may also be used as further agent for coupling to the anti-LYPD3 antibody. Furthermore, the antibody may be engineered so as to enhance its ability to activate the patient's immune response, in particular the ability to activate ADCC (antibody-dependent cell-mediated cytotoxicity) and/or CDC (complement dependent cytotoxicity). For example, this may be achieved by optimizing the amino acid sequence and/or the glycosylation pattern of the antibody, in particular of its constant regions.

For use as detection agent in diagnosis, prognosis and/or monitoring of a disease, the antibody preferably is coupled to a labeling agent which is capable of producing a detectable signal. In particular, said labeling agent may be a radionuclide, a fluorophore or an enzyme.

### FIGURES

FIG. 1 shows the biosynthesis pathways of the Tn and TF carbohydrate antigens as well as their structures. In the structures, a square represents GalNAc, a circle represents Gal, and a diamond represents a sialic acid.

FIG. 2 shows glyco-dependent target binding of anti-LYPD3 antibodies to A) LYPD3-ECD or B) the STP-rich C-terminal part of the ECD (LYPD3-STP-ECD). Binding to equimolar amounts of antigens (35 nM) was assessed in an antigen ELISA using 5 μg/ml of a-LYPD3 mAbs. To control antigen coating to the ELISA plates, aLYPD3 pAb or aMBL Ab were used. Proteins were expressed in and purified from transfected NM-F9 cells with the respective O-glycosylation pattern (F9, O-glyc.), de-O-glycosylated proteins were produced by enzymatic digestion of these NM-F9 proteins (F9 de-O-glyc.). Binding to LYPD3-STP-ECD recombinantly expressed and purified from O-glycosylation deficient CHO cells was also tested (aglyc.). A high O.D. signal indicates strong binding of the antibody to the antigen.

FIG. 3 shows binding of anti-LYPD3 clones to the different glycosylated LYPD3 variants. Binding to equimolar amounts of antigens (35 nM) was assessed in an antigen ELISA using the indicated concentrations of aLYPD3 mAbs. aLYPD3 pAb or aMBL Ab were used as coating controls, aTF and aTn antibodies as glycosylation controls. TF- and Tn-carrying proteins were recombinantly expressed and purified from NM-F9 cells, de-O-glycosylated proteins resulted from enzymatic digestion of NM-F9-derived proteins, and Tn proteins by de-galactosylation of NM-F9-derived proteins as described in Example 1. A high O.D. signal indicates strong binding of the antibody to the antigen.

FIG. 4 shows the analysis of anti-LYPD3 clones tested for glycan binding by ELISA. Binding to PAA-conjugated glycans coated with 10 μg/ml was assessed in an antigen ELISA using 5 μg/ml of aLYPD3 mAbs; aTn, aTF and a-sTn mAbs were used to control coating of the indicated 4 glycans to the ELISA plates. A high O.D. signal indicates strong binding of the antibody to the antigen.

FIG. 5 shows binding curves of anti-huLYPD3 antibodies to NM-F9-derived LYPD3-STP in ELISA. Anti-LYPD3 antibodies were titrated on ELISA plates coated with LYPD3-STP-ECD target protein. High O.D. signal indicates strong binding of the antibody to the antigen.

FIG. 6 shows glyco-dependent antigen binding of anti-LYPD3 antibodies to LYPD3-ECD in comparison to aLYPD3 mAb, aTF and aTn. Binding to equimolar amounts of antigens was assessed in an antigen ELISA using a-LYPD3 mAbs. Proteins were expressed in and purified from transfected NM-F9 or NM-H9D8 cells with the respective O-glycosylation patterns (NM-F9: mainly TF; H9D8: mainly sTF and TF). De-sialylated proteins were produced by enzymatic treatment proteins with sialidase (H9D8, de-sialyl.: mainly TF) and de-galactosylated proteins by treatment with galactosidase (NM-F9, Tn-glyc.: mainly Tn). A high O.D. signal indicates strong binding of the antibody to the antigen.

FIG. 7 shows binding of aLYPD3 clones to cellular O-glyc. and de-O-glyc LYPD3. Cell lines with different LYPD3 expression (LYPD3-F9=high O-glycosylation, high LYPD3; NM-F9=high O-glycosylation, low LYPD3; LYPD3-HEK-O-glyc KO=no O-glycosylation, high LYPD3) were stained with 1 μg/ml of aLYPD3 clones, control aLYPD3 pAb and mAb and aTF mAb. Shown is signal-to-noise ratio of stained cells: MFI (median fluorescence intensity) of stained cells divided by MFI of isotype-stained cells.

FIG. 8 shows titration of aLYPD3 clones compared to control aLYPD3 mAb on LYPD3-F9 cells expressing high levels of O-glyc. LYPD3. Cells were stained with different concentrations of aLYPD3 clones and aLYPD3 mAb control and detected with fluorophore-coupled anti-human-IgG secondary reagent. Shown is the MFI of live cells.

FIG. 9 shows titration of aLYPD3 clones on LYPD3-F9 cells expressing high levels of O-glyc. LYPD3. Cells were stained with different concentrations of aLYPD3 clones and detected with fluorophore-coupled anti-human-IgG secondary reagent. Shown is the MFI of live cells.

FIG. 10 shows binding of aLYPD3 clones to normal human epithelial mammary cells (HMEC). HMEC from healthy donors were stained with 10 μg/ml aLYPD3 clones and fluorophore-coupled anti-human-IgG secondary reagent. Expression of LYPD3 and TFa was confirmed using control aLYPD3 pAb and mAb and aTF mAb. Shown is signal-to-noise ratio of stained cells: MFI (median fluorescence intensity) of stained cells divided by MFI of isotype-stained cells.

FIG. 11 shows binding of aLYPD3 clones to Caov-3 cells, ZR-75-1 cells with and without sialidase treatment, and MDA-MB-231 cells after sialidase treatment. (A) Caov-3 cells were stained with 10 μg/ml aLYPD3 clones and fluorophore-coupled anti-human-IgG secondary reagent. (B) ZR-75-1 cells were treated with sialidase (30 min with 5 mU/ml) or not, washed and stained with 10 μg/ml aLYPD3 clones and fluorophore-coupled anti-human-IgG secondary reagent. (C) MDA-MB-231 cells were treated with sialidase (30 min with 5 mU/ml), then stained with 10 μg/ml aLYPD3 clones and fluorophore-coupled anti-human-IgG secondary reagent. Expression of LYPD3 and TFa was confirmed using control aLYPD3 pAb and mAb and aTF mAb. Shown is signal-to-noise ratio of stained cells: MFI (median fluorescence intensity) of stained cells divided by MFI of isotype-stained cells.

FIG. 12 shows the capacity of aLYPD3 clone 17B3 to inhibit proliferation of LYPD3-F9 cells. Cells were incubated with different concentrations of aLYPD3 antibody 17B3 together with constant amounts of protein G-MMAE. Proliferation was measured after 4 days. Irrelevant human IgG1 control shows no inhibition. Proliferation is shown in % relative to a medium control without antibody based on luminescent signals.

FIG. 13 shows binding of selected anti-LYPD3 clones to healthy esophagus tissue sections in comparison to aLYPD3 pAb control antibody.

FIG. 14 shows binding of humanized aLYPD3 antibodies to cellular O-glyc. and de-O-glyc LYPD3 determined by flow cytometry. (A) Cell lines with different LYPD3 expression and O-glycosylation (LYPD3-ECD HEK O-glyc KO=no O-glycosylation, high LYPD3; mock F9 O-glyc.=high O-glycosylation with TF, low LYPD3; LYPD3-ECD F9=high O-glycosylation with TF, high LYPD3) were stained with 10 μg/ml of humanized aLYPD3 antibodies (21E9-219 to 21E9-228), the parental antibody (parental 21E9), and control antibodies against LYPD3 (aLYPD3 pAb and aLYPD3 mAb). (B) Cell line NM-F9 expressing LYPD3 with TF O-glycosylation was stained different concentrations (0.1 μg/ml and 10 μg/ml) of the humanized antibodies. Shown is signal-to-noise ratio of stained cells: MFI (median fluorescence intensity) of stained cells divided by MFI of isotype-stained cells.

FIG. 15 shows binding of humanized aLYPD3 antibodies to O-glycosylated LYPD3-STP-ECD in F9 cells. Binding to equimolar amounts of antigens was assessed in an antigen ELISA using the indicated concentrations of the humanized aLYPD3 mAbs and the parental aLYPD3 mAb. Proteins were expressed in and purified from transfected NM-F9 cells and carried TF O-glycosylation. A high O.D. signal indicates strong binding of the antibody to the antigen.

FIG. 16 shows glycan specific binding of humanized aLYPD3 antibodies. Binding to equimolar amounts of antigens was assessed in an antigen ELISA using the indicated concentrations of aLYPD3 mAbs. Proteins were expressed in and purified from transfected NM-F9 cells with the respective O-glycosylation patterns and not treated (F9 O-glyc.: mainly TF), de-galactosylated (F9 O-glyc. de-gal.: mainly Tn) or de-glycosylated (F9 de-O-glyc.: no O-glycosylation). A high O.D. signal indicates strong binding of the antibody to the antigen.

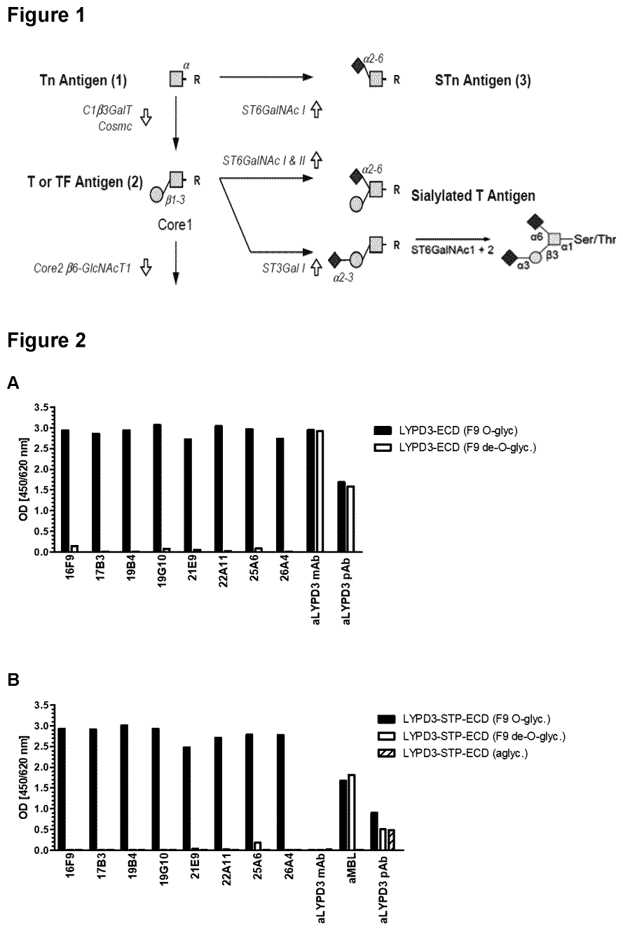

### EXAMPLES

### Example 1: Generation of Antigens

Different O-glycosylation variants of human LYPD3 were generated for an optimized selection of antibodies specifically binding to human LYPD3 with tumor-associated glycosylation pattern. Because the C-terminal part of the huLYPD3 extracellular domain (aa 234-303) contains a large number of Ser/Thr/Pro residues and thus a high density of O-glycosylation sites, this huLYPD3 aa sequence was chosen as target structure (“LYPD3-STP”) for antibody generation with a high potential to yield glyco-dependent anti-LYPD3 antibodies in antibody generation approaches described in Example 2.

Different cell lines were transfected with DNA encoding the huLYPD3 extracellular domain (ECD; aa 31-303), especially the C terminal Ser/Thr/Pro-rich domain of huLYPD3 (STP-ECD; aa 234-303), to generate cells expressing soluble LYPD3-ECD or LYPD3-STP-ECD. Soluble protein constructs were recombinantly expressed as fusion proteins, e.g., with a tandem version of the Strep-tag® (Twin-Strep-tag®, IBA, Germany), maltose binding lectin (termed here MBL) or polyhistidine tag to enable purification from cell supernatant by affinity chromatography and detection of protein via the fusion part independent of LYPD3. In addition, different cell lines were transfected with DNA encoding the complete human LYPD3 propeptide sequence (aa 31 to 346) or the C terminal Ser/Thr/Pro-rich domain of huLYPD3 propeptide (aa 234-346) together with C-terminal EGFP fusion, to generate cells expressing membrane-bound LYPD3 or LYPD3-STP on the cell surface, respectively.

The cancer cell line derived NM-F9 and NM-H9D8 cells were used for expression of human LYPD3 and LYPD3-STP proteins with tumor-associated O-glycosylation pattern: while NM-H9D8 cells provide a glycosylation of LYPD3 with a high degree of sialylation (mainly sTF and sTn), the sialylation deficient NM-F9 cells predominantly produce LYPD3 carrying TF and lower amounts of Tn. O-glycosylation deficient (O-glyc KO) CHO (CH-O-delete; GlycoDisplay Aps, Copenhagen, Denmark) or HEK cells (HEK GALE/GALEK2 KO, Kerafast, Inc., Boston, MA, USA) were used for production of LYPD3 variants without O-glycosylation.

Tn-LYPD3-STP-ECD was either recombinantly expressed in HEK cells, deficient in producing core 1 O-glycan extension (HEK293 cosmc KO, Glycodisplay) or produced via enzymatic digestion of NM-F9-derived LYPD3-STP-ECD with a β-galactosidase (GalactEXO, Genovis) to remove galactose residues from TF structures resulting in GalNAc linked to serine or threonine, referred to as Tn antigen.

Soluble LYPD3 proteins without O-glycosylation either were directly purified from supernatants of transfected O-glycosylation deficient CHO cells or were generated from proteins purified from NM-H9D8 or NM-F9 transfectants by enzymatic digestion. The latter was performed applying either soluble sialidases, O-glycosidase and α-N-acetylgalactosaminidase or immobilized β-galactosidase and GalNAcase enzymes in microspin columns in order to remove sialic acids (α2-3, α2-6 & α2-8), O-glycans of core 1 and GalNAc residues on glycoproteins (SialExo, Oglyzor, GalNAcEXO, GalactEXO all Genovis).

Binding of anti-huLYPD3 antibodies to purified soluble proteins was assessed in ELISA assays as described in Example 3, while binding to differently glycosylated membrane-bound LYPD3 was analyzed using flow cytometry.

### Example 2: Generation of Anti-LYPD3 Antibodies

Monoclonal antibodies specifically recognizing the STP-rich domain of huLYPD3 with tumor-associated glycosylation pattern were either generated by phage display technology or via immunization of animals. In any case huLYPD3 carrying O-glycan structures produced by cancer cells in form of the proteins and cells described in Example 1 was an integral part of the antibody generation procedure. All antibodies were selected to bind human LYPD3 in an O-glycosylation-dependent manner.

HybriFree technology was used to isolate LYPD3-specific antibodies from splenocytes of chickens and rabbits immunized either with purified LYPD3-ECD or STP-rich ECD part (LYPD3-STP-ECD) from transfected NM-F9 cells and boosted with LYPD3-ECD. Spleen cells were isolated after final immunization from animals with confirmed antigen-specific antibody response in blood serum or chicken egg yolk preparations which was assessed by flow cytometry (similar to procedure described in Example 4) and/or ELISA assays (similar to procedure described in Example 3). Splenic B cells with antigen-specificity for O-glycosylated LYPD3-STP were enriched initially by depletion of cells showing unwanted protein binding. This included binding to LYPD3-STP-ECD without glycosylation and/or binding to recombinantly expressed protein fusion partners without any LYPD3 sequence present, and/or to an irrelevant glycoprotein similarly produced as O-glycosylated LYPD3-STP-ECD and by this containing identical protein fusion partners as well as similar glycan structures bound to non-homologous protein sequences. For this negative selection step, spleen cells were first incubated with off-target proteins immobilized on microtiter plates and/or present in the panning solution. Subsequently unbound cells were transferred to plates coated with O-glycosylated LYPD3-STP-ECD or LYPD3-ECD protein purified from NM-F9 cells to capture splenic B cells with target-specificity. cDNA of antibody variable domains was amplified from the captured cells, cloned into a plasmid with separate expression cassettes for the IgG heavy and light chain, respectively, for construction of a combinatorial human IgG1 encoding library in a mammalian expression vector. Plasmid DNA from resulting antibody library pools was transfected into CHO cells for transient production of chimeric antibodies. Antibody mini-pool cell culture supernatants were tested for target-specific binding in ELISA with various on-and off-target proteins and additionally by flow cytometry on LYPD3-transfected HEK O-glyc KO cells and/or LYPD3-transfected F9 vs. non-transfected NM-F9 cells. Proteins used for ELISA screening included O-glycosylated LYPD3-ECD and LYPD3-STP-ECD, both expressed in and purified from NM-F9 cells, as well as LYPD3-ECD and LYPD3-STP-ECD, both without glycosylation. Single clones were generated from antibody pools that showed specific binding to O-glycosylated LYPD3-STP. VH and VL cDNAs of antigen-specific single clones were sequenced, and antibodies with unique sequences were expressed in CHO cells. Antigen-specific antibody production in single clone supernatants was confirmed by ELISA as well as flow cytometry before antibody purification.

The following glycosylation-specific anti-huLYPD3 antibodies were obtained by immunization of chicken or rabbit:

| | | | | |
|---|---|---|---|---|
| 16F9 | 17B3 | 19B4 | 19G10 | 21E9 |
| 22A11 | 25A6 | 26A4 | | |

Fully human glyco-specific anti-huLYPD3 antibodies were isolated by phage display technology form the naïve human antibody library of Yumab (YUMAB GmbH, Braunschweig, Germany) Briefly, bacteriophages with antigen-specificity for O-glycosylated LYPD3-STP were enriched from the library by 3 rounds of antibody selection using different strategies that combine subsequent panning reactions on purified proteins and on cells. In each step positive selection of scFv-producing phages was achieved either by binding to O-glycosylated LYPD3-STP-ECD or LYPD3-ECD, both produced from NM-F9 cells and immobilized on microtiter plates. For competition, purified recombinantly expressed protein fusion partner without LYPD3 sequence and both LYPD3-ECD and LYPD3-STP-ECD without O-glycosylation were present in the panning solution to deplete antibodies recognizing these structures. For some strategies, 2^{nd} and/or 3^{rd} enrichment steps were done by incubation with LYPD3-STP transfected NM-F9 cells after depletion of phages binding to non-transfected NM-F9 cells. For other strategies, binding to LYPD3-transfected NM-F9 cells or to neuraminidase-treated Caov-3 cells, an ovarian adenocarcinoma cell line expressing high levels of LYPD3 and TFa glycan, were used for antibody selection after depleting non-transfected NM-F9-binding phages. Soluble scFv antibodies were produced from single clones of each selection strategy and screened by ELISA on LYPD3-ECD and LYPD3-STP-ECD proteins with and without O-glycosylation. scFv antibodies with glyco-dependent LYPD3 binding in ELISA were also tested by flow cytometry for specific binding to NM-F9 cells transfected with LYPD3 or LYPD3-STP as well as neuraminidase treated Caov-3 cells. After sequencing of VH and VL DNA of clones specifically recognizing O-glycosylated LYPD3-STP in ELISA and on LYPD3-positive cells, unique antibody VH and VL sequences were cloned into murine IgG2a expression vector for transfection of HEK cells.

The following glycosylation-specific anti-huLYPD3 antibodies were obtained by isolation from a phage display library of human antibodies:

| | | | | |
|---|---|---|---|---|
| YU912-B09 | YU912-G05 | YU912-B06 | YU913-B01 | YU912-D06 |
| YU912-A03 | YU912-C07 | YU912-G08 | YU912-G07 | |

All selected antibodies were purified via protein A affinity chromatography from mammalian cell culture supernatant. Purity and integrity of mAbs was confirmed with SDS-PAGE and analytical SEC.

For some experiments, VH and VL sequences of anti-human LYPD3 antibodies were cloned also in a murine IgG1 expression vector for purification of chimeric antibodies with mouse IgG backbone. Both, purified chimeric anti-LYPD3 expressed as human IgG1 or murine IgG1 with the same VH and VL combination show similar binding characteristics on soluble as well as membrane bound proteins as confirmed in ELISA and flow cytometry assays.

### Example 3: Antigen ELISA with On- and Off-Target Controls

Antibodies were analyzed in antigen ELISA assays for specific binding to LYPD3 carrying tumor-associated glycans.

Briefly, differently glycosylated and non-glycosylated protein antigens or glycans conjugated to carbohydrate-polyacrylamide conjugates (carbohydrate PAA-conjugates, GlycoNZ) were coated to 96-well plates overnight, unspecific binding was blocked and test antibody samples were added. In some assays an anti-huLYPD3 huIgG1 control monoclonal Ab was used, that was purified from CHO cell supernatant after expression of the antibody sequence derived from patent application WO 2011/070088 A1 (SEQ ID NOs: 51 and 52 of WO 2011/070088) (“aLYPD3 mAb”). This control anti-huLYPD3 antibody binds to the extracellular domain of human LYPD3 in a glycosylation-independent manner. To control protein antigen coating to the ELISA plates anti-huLYPD3 rabbit polyclonal antibody (R&D Systems, cat. no. AF5428) (“aLYPD3 pAb”) or a-MBL antibody (Novusbio) (“aMBL”) for constructs expressed as MBL fusion protein were used. Antibodies recognizing specific glycan structures were used to detect glycosylation of the coated proteins. Anti-TFa (“aTF”) antibody clone HH8 was kindly provided by Prof. Clausen, University of Copenhagen. Antibodies specifically recognizing Tn (“aTn”) were either purchased from SBH Sciences or also provided by Prof. Clausen, University of Copenhagen (clone 5F4). Afterwards, peroxidase conjugated anti-IgG secondary antibody was added, followed by 3,3′,5,5′-tetramethylbenzidine (TMB) substrate reaction. Antibody binding rates were determined by measurement absorbance at 450 nm and 620 or 630 nm as reference wavelength using a multimode microplate reader (PerkinElmer EnSpire 2300 or Tecan Spark).

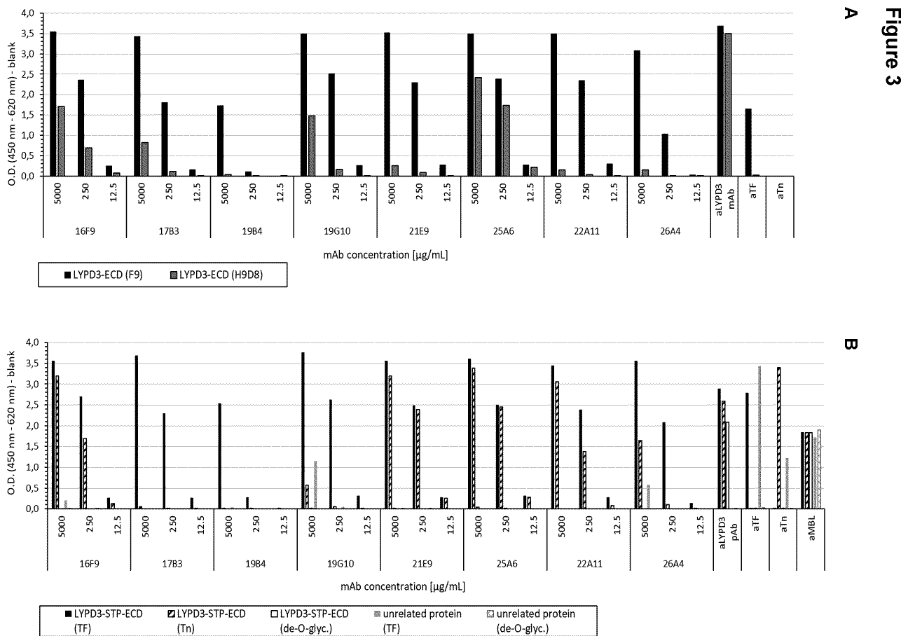

Glyco-dependent binding of the generated a-huLYPD3 antibodies to the STP-rich domain of huLYPD3 was assessed in an antigen ELISA (FIG. 2). All antibodies showed significant binding to O-glycosylated LYPD3-STP-ECD and also O-glycosylated LYPD3-ECD protein, that contained the LYPD3-STP sequence as well, confirming that the epitope recognized by these antibodies is present in huLYPD3 aa sequence R234-H303. Absence of O-glycosylation on the tested LYPD3 proteins, achieved either by enzymatic de-O-glycosylation or by recombinant expression and purification from O-glycosylation deficient cells, abrogates binding of the a-LYPD3 antibodies. This clearly shows that an O-glycosylated LYPD3 epitope is required for antibody binding and thus confirms glyco-dependency of LYPD3-binding for all selected antibodies. However, the a-LYPD3 control mAb was able to recognize LYPD3-ECD independent of O-glycosylation and was not able to bind any of the LYPD3-STP-ECD variants. This antibody hence binds glycosylation-independently to a LYPD3 epitope located in aa 31-233 of huLYP3.

Furthermore, anti-huLYPD3 antibody clones seem to elicit diverse fine specificities regarding the type of LYPD3 O-glycosylation. This is evident from varying degrees of recognition of LYPD3 proteins expressed and purified from NM-H9D8 providing a high degree of sialylation, carrying mainly sTF and some TF, of NM-H9D8-expressed LYPD3 treated with sialidase, carrying mainly TF, and of de-galactosylated LYPD3-STP-ECD carrying mainly Tn-glycosylation (Tn glyc.). The relative amount of the different O-glycosylation structures on LYPD3 was determined for LYPD3 expressed in different cells lines with and without enzymatic treatment:

TABLE 1
Glycosylation structures on LYPD3

| | % TF | % Tn | % sTF | % sTn |
|---|---|---|---|---|
| LYPD3 (NM-F9) | 96.6 | 3.4 | 0 | 0 |
| LYPD3 (NM-F9, de-gal.) | 6.6 | 93.4 | 0 | 0 |
| LYPD3 (NM-H9D8) | 38.9 | 2.0 | 52.3 | 6.8 |
| LYPD3 (NM-H9D8, de-sial.) | 91.1 | 8.9 | 0 | 0 |

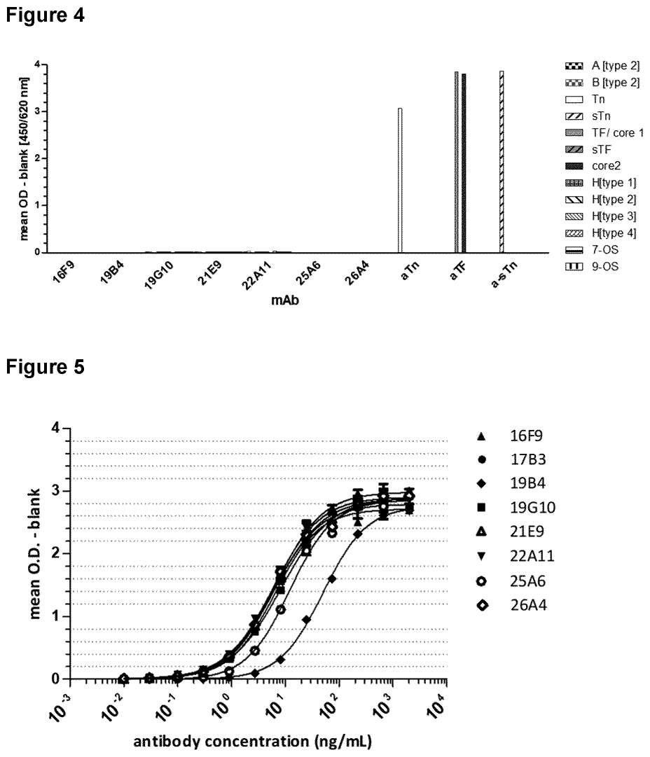

The analyzed antibodies showed strong, highly specific binding to O-glycosylated LYPD3, but no significant binding to unrelated O-glycosylated proteins (FIG. 3) or pure carbohydrate antigens (FIG. 4). Thus, binding of the anti-LYPD3 antibody clones also specifically relies on the LYPD3 protein backbone.

Anti-LYPD3 antibodies were compared regarding their binding affinity to glycosylated LYPD3-STP-ECD (expressed in NM-F9 cells). All anti-LYPD3 antibodies showed dose-dependent binding to the O-glycosylated LYPD3-STP target structure with high affinity and EC50 values between 4×10⁻¹⁰ to 4×10⁻¹¹M (FIG. 5).

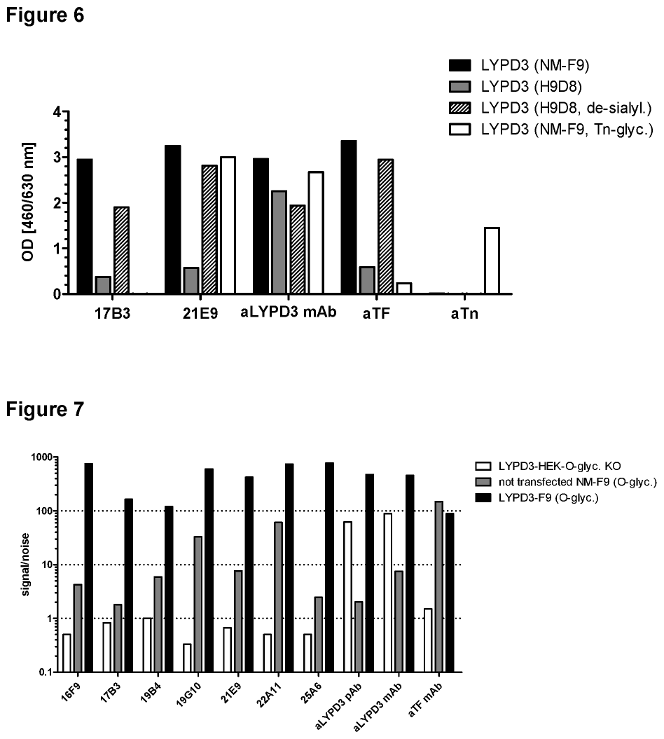

Anti-LYPD3 clones 17B3 and 21E9 were further compared regarding their binding affinity to LYPD3 carrying TF (expressed in NM-F9 cells, or in NM-H9D8 and treated with sialidase), LYPD3 carrying mainly sTF (sialylated TF) and some TF (expressed in NM-H9D8 cells), and LYPD3 carrying Tn (expressed in NM-F9 cells and treated with galactosidase). Both clones showed strong binding to TF-glycosylated LYPD3, while 21E9 additionally recognizes Tn-glycosylated LYPD3 (FIG. 6).

### Example 4: Binding to O-Glyc. and De-O-Glyc. LYPD3 Expressed on Cell Lines

Binding of aLYPD3 clones to cell lines with different glycosylation status of LYPD3 was tested. LYPD3-transfected NM-F9 (LYPD3-F9), expressing LYPD3 carrying non-sialylated O-glycans, not transfected NM-F9 (corresponding WT cells), expressing endogenous level of LYPD3, and LYPD3-transfected HEK-O-glyc KO (LYPD3-HEK-O-glyc. KO) expressing LYPD3 without O-glycans, were stained with aLYPD3 clones and detected via fluorophore-coupled anti-human-IgG secondary reagent. Expression of LYPD3 and TFa was confirmed using control aLYPD3 pAb and mAb and aTF mAb. To determine background staining, an irrelevant hIgG1 control was included. DAPI was used to discriminate live from dead cells. Cells were analyzed using a Canto II (BD) flow cytometer.

The results showed that all aLYPD3 clones bound to O-glycosylated LYPD3 on LYPD3-F9 and NM-F9 but not to non-O-glycosylated LYPD3 on LYPD3-HEK-O-glyc KO cells. Binding to NM-F9 cells was comparable to stainings with aLYPD3 pAb and mAb (FIG. 7).

### Example 5: Titration of aLYPD3 Clones on LYPD3-F9 Cells

To compare binding affinities of different aLYPD3 clones, LYPD3-F9 cells expressing high levels of O-glyc. LYPD3 were stained with different concentrations of aLYPD3 clones and bound antibodies were detected with fluorophore-coupled anti-human-IgG secondary reagent. To determine background staining, an irrelevant hIgG1 control was included and DAPI was used to discriminate live from dead cells. Cells were analyzed using a Canto II (BD) flow cytometer.

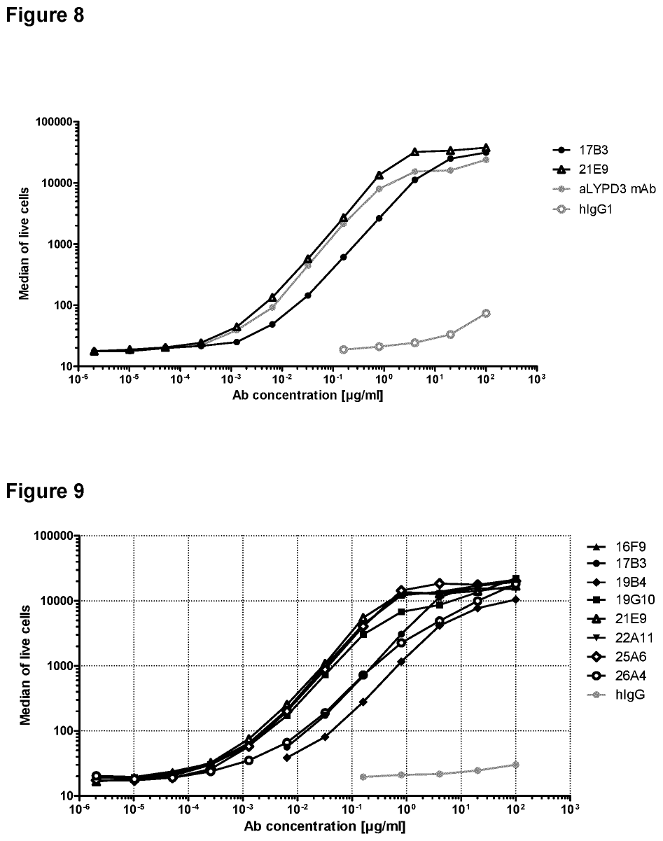

All aLYPD3 clones showed dose-dependent binding to LYPD3-F9 cells (FIGS. 8 and 9). Binding of aLYPD3 clones was in the same range as the aLYPD3 mAb control (FIG. 8). The clones 16F9, 19G10, 21E9, 22A11 and 25A6 showed very similar binding affinities which were slightly higher than those of clones 17B3, 19B4 and 26A4 (FIG. 9).

### Example 6: aLYPD3 Clones Do Not Bind to LYPD3 Expressed on Normal Human Epithelial Mammary Cells

To demonstrate that aLYPD3 clones do not bind to LYPD3 glycoforms mainly found on normal human cells, human epithelial mammary cells (HMEC) from healthy donors were stained with aLYPD3 clones and detected via fluorophore-coupled anti-human-IgG secondary reagent. Expression of LYPD3 and TFa was confirmed using control aLYPD3 pAb and mAb and aTF mAb. To determine background staining, an irrelevant hIgG1 control was included. DAPI was used to discriminate live from dead cells. Cells were analyzed using a Canto II (BD) flow cytometer.

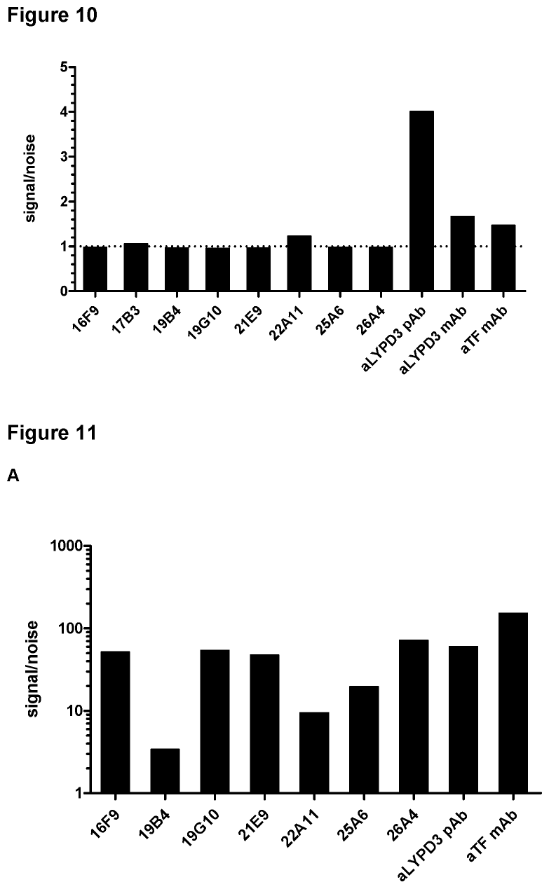

HMEC expressed medium levels of LYPD3 (˜40% LYPD3 positive) but almost no TFa as determined with control antibodies aLYPD3 pAb and mAb and aTF mAb. None of the aLYPD3 clones showed binding to LYPD3 expressed on HMEC (FIG. 10).

### Example 7: Binding of aLYPD3 Clones to Tumor Cell Lines

To examine binding of aLYPD3 clones to tumor cell lines, different cell lines with varying expression levels for LYPD3 and TFa were chosen: Caov-3 (ovarian adenocarcinoma cell line, LYPD3+), ZR-75-1 (breast carcinoma cell line, LYPD3+) and MDA-MB-231 (breast adenocarcinoma cell line, LYPD3−). MDA-MB-231 and ZR-75-1 were treated with sialidase to remove sialic acid from the cell surfaces. Tumor cell lines were stained with aLYPD3 clones and binding was detected using fluorophore-coupled anti-human-IgG secondary reagent. Expression of LYPD3 and TFa was confirmed using control aLYPD3 pAb and aTF mAb. To determine background staining, an irrelevant hIgG1 control was included. DAPI was used to discriminate live from dead cells. Cells were analyzed using a Canto II (BD) flow cytometer.

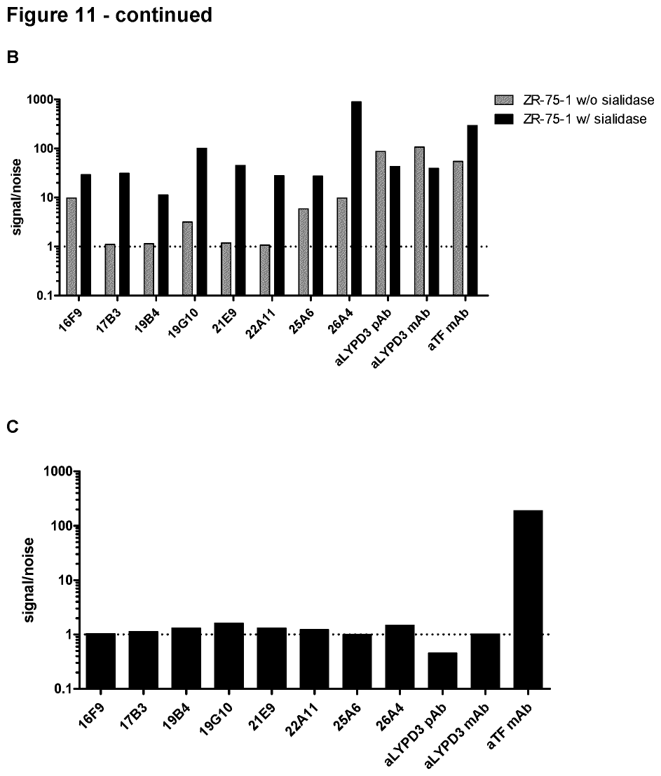

All tested aLYPD3 clones bound to Caov-3, a cell line expressing high level of LYPD3 and TFa. Binding was comparable to aLYPD3 pAb (FIG. 11A). The ZR-75-1 cell line expresses lower level of TFa compared to Caov-3 and binding of aLYPD3 clones to ZR-75-1 was thus investigated prior and after sialidase treatment to expose further TFa molecules. All aLYPD3 clones bound to sialidase treated ZR-75-1 (FIG. 11B). Some aLYPD3 clones (16F9, 19G10, 25A6 and 26A4) showed also binding to untreated ZR-75-1. aLYPD3 clones did not bind to MDA-MB-231 cells which displayed high levels of TFa after sialidase treatment but did not express LYPD3 (FIG. 11C).

### Example 8: Inhibition of Proliferation Using Protein G-Drug Conjugated aLYPD3 Antibodies

In order to investigate the potential of aLYPD3 clones to deliver cytotoxic drugs into target cells, protein G-drug conjugate assays were performed. In flat bottom 96-well plates, 5000 LYPD3-F9 cells per well were seeded in the presence of indicated test antibody dilutions as well as constant concentrations of Protein G preloaded with the toxin MMAE (Protein G-MMAE, Levena Biopharma). After incubation for 4 days, cell viability was assessed using CellTiter-Glo® Luminescent Cell Viability Assay (Promega) according to the manufacturer's instructions and analyzed in a microplate reader TECAN Infinite F200 (Tecan). Proliferation in percent was calculated relative to a medium control without antibody based on luminescent signals.

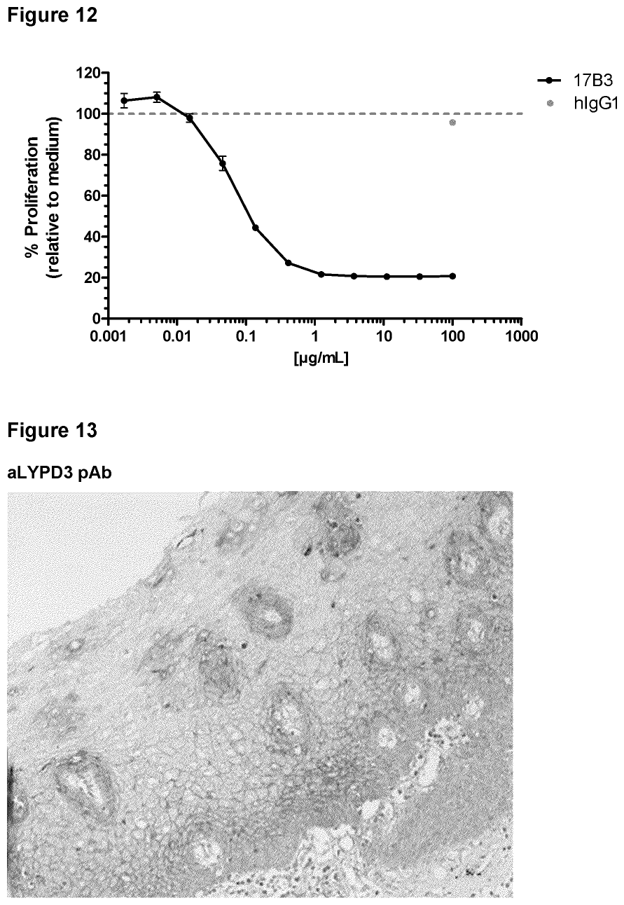

The results demonstrated that aLYPD3 clone 17B3 conjugated to protein G-MMAE inhibits proliferation of LYPD3-F9 in a dose-dependent manner indicating effective internalization of the aLYPD3 clone (FIG. 12).

### Example 9: Tissue Binding of Glyco-Specific Anti-LYPD3 Antibodies

Formalin-fixed paraffin embedded tissue sections were stained with 10 μg/ml aLYPD3 clones and detected using either HRP labelled polymer conjugated to anti-mouse or anti-human secondary antibodies. A commercial polyclonal aLYPD3 antibody was included to detect protein levels of LYPD3 in the respective tissues.

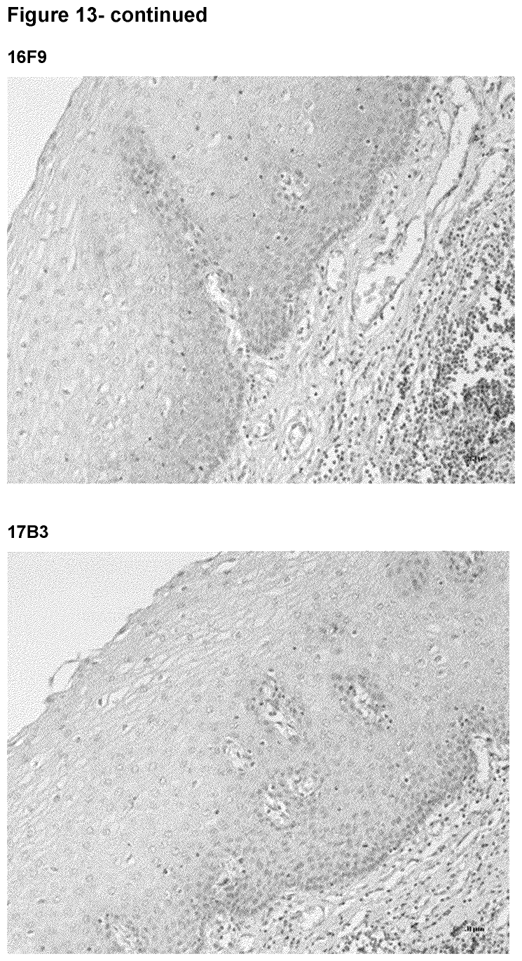

Staining of cancer tissue sections showed binding of aLYPD3 clones to head and neck squamous cell carcinoma of different origins, esophageal carcinoma, breast carcinoma, cervical carcinoma and skin carcinoma. Importantly, the reactivity to healthy tissue was absent or reduced as compared to the protein-specific control aLYPD3 pAb shown in FIG. 13 and Table 2. In conclusion, aLYPD3 clones were found to react with several cancer tissue sections, but not their healthy counterparts. In particular, 17B3 and 21E9 clones were shown to interact strongly with SCC cancer tissue sections of the anal area/rectum, oral cavity, lip, bucca cavioris, nose, penis, vulva, epiglottis, tongue and skin (data not shown).

TABLE 2
Binding of aLYPD3 clones to healthy tissue

| | Esophagus | Cervix | Tonsil |
|---|---|---|---|
| aLYPD3 pAb | + | + | + |
| 16F9 | +/− | +/− | − |
| 17B3 | − | − | − |
| 19B4 | − | − | − |
| 19G10 | − | − | − |
| 22A11 | − | − | − |
| 26A4 | − | − | − |

Binding of aLYPD3 clones to healthy tissue in comparison to pAb control antibody is shown, classified according to staining intensities: + medium to strong staining, +/− weak staining, − no staining observed.

In particular, 17B3 and 21E9 clones were shown to interact strongly with SCC tissue sections of the anal area/rectum, oral cavity, lip, bucca cavioris, nose, penis, vulva, epiglottis, tongue and skin.

### Example 10: Humanization of the Rabbit Heavy and Light Chain Variable Regions of Anti-huLYPD3 Antibody 21E9

The nucleic acid sequences coding for the rabbit heavy and light chain variable regions of the monoclonal anti-huLYPD3 antibody 21E9 (SEQ ID NO: 39 and 40) were ligated to the sequences of the human constant γ1 region (CH) and the human constant κ region (CL), respectively.

On the basis of these chimeric clones, humanized antibodies were constructed. To this end, point mutations were introduced into the nucleic acid sequences of the rabbit framework regions of VH and VL in order to generate the corresponding human framework regions. The target human framework regions were selected from a database generated from NCBI GenBank entries (cleaned to remove incomplete and non-human sequences) to include both germline and mature antibody frameworks. In particular, the most related framework regions were chosen from the library depending on their overall sequence similarity and their CDR loop classification. All data obtained were considered to design a set of different variable sequences of humanized variable light and variable heavy chains of the parent rabbit antibody. Some of the variants contain back-mutations to the rabbit sequence on critical positions. The eight humanized variants of the light chain variable region were cloned in a κ-chain vector and the eight humanized variants of the heavy chain variable region were cloned in a γ1-chain vector.

Antibodies comprising different combinations of the obtained heavy and light chains (in total 64 antibody variants) were transiently expressed and screened for their expression and LYPD3 binding in ELISA according to example 3. The following humanized antibody heavy and light chains variable regions were selected for further analysis.

TABLE 3
Humanized antibody heavy and light chains variable regions

| VH | SEQ ID NO | VL | SEQ ID NO |
|---|---|---|---|
| 1 | 138 | 1 | 143 |
| 2 | 139 | 2 | 144 |
| 3 | 140 | 4 | 145 |
| 6 | 141 | 8 | 146 |
| 7 | 142 | | |

Nine selected antibody combinations of humanized VH and VL sequences were produced as human IgG1/kappa by transient expression in CHO cells and purified by Protein A chromatography.

TABLE 4
Humanized antibodies tested

| Antibody production lot number | VH | VL |
|---|---|---|
| 21E9-219 | 2 | 2 |
| 21E9-220 | 3 | 2 |
| 21E9-221 | 6 | 2 |
| 21E9-222 | 7 | 2 |
| 21E9-224 | 7 | 1 |
| 21E9-225 | 7 | 4 |
| 21E9-226 | 7 | 8 |
| 21E9-227 | 1 | 4 |
| 21E9-228 | 3 | 4 |

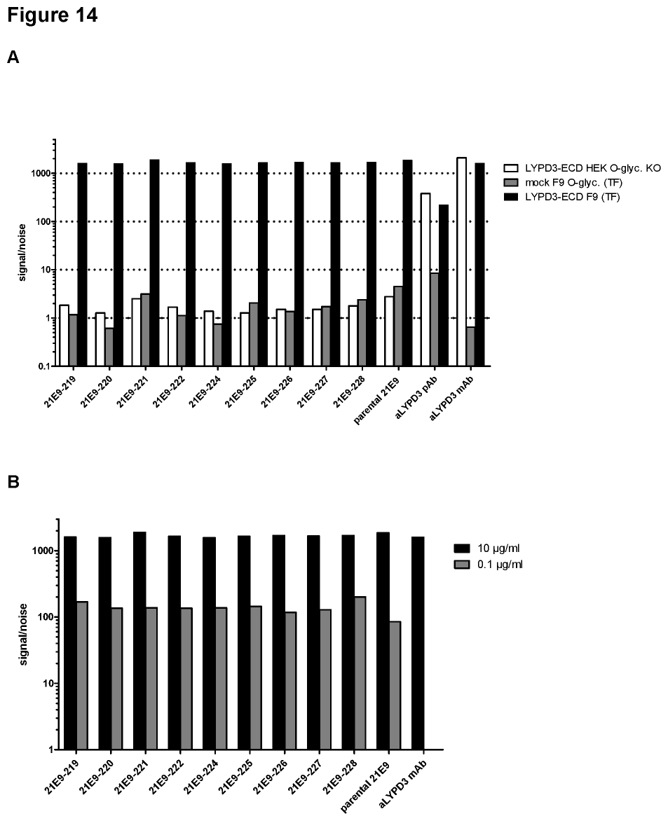

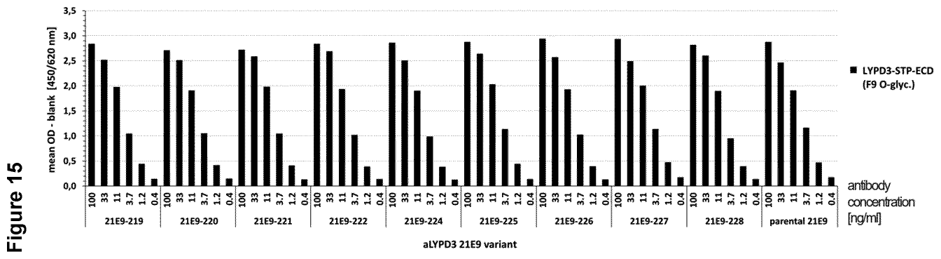

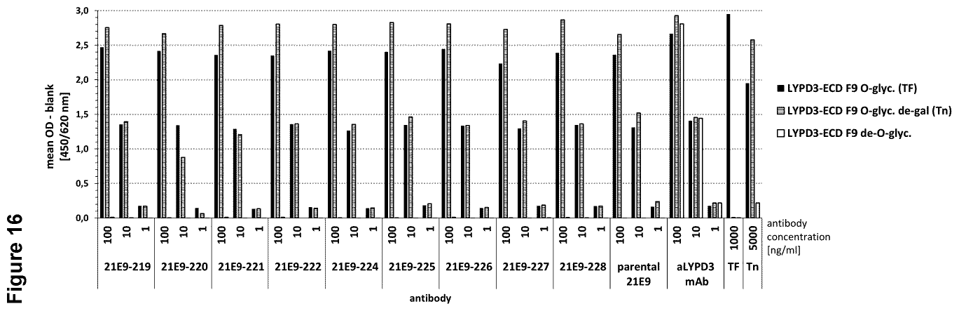

Their binding specificity and affinity were analyzed in ELISA and flow cytometry assays according to example 3 and 4 and compared to the parental chimeric antibody. Data are summarized in FIGS. 14, 15 and 16. All humanized antibody variants showed the same binding affinity and specificity to LYPD3 carrying TF or Tn as the parental antibody 21E9.

### Identification of the Deposited Biological Material

The cell line DSM ACC 2606 was deposited on the date indicated in the following table at the DSMZ—Deutsche Sammlung von Mikroorganismen und Zellkulturen GmbH, Mascheroder Weg 1b, 38124 Braunschweig (DE) by Nemod Biotherapeutics GmbH & Co. KG, Robert-Rössle-Str. 10, 13125 Berlin (DE). Glycotope is entitled to refer to this biological material since it was in the meantime assigned from Nemod Biotherapeutics GmbH & Co. KG to Glycotope GmbH.

The cell lines DSM ACC 2806, DSM ACC 2807 and DSM ACC 2856 were deposited at the DSMZ—Deutsche Sammlung von Mikroorganismen und Zellkulturen GmbH, Inhoffenstraße 7B, 38124 Braunschweig (DE) by Glycotope GmbH, Robert-Rössle-Str. 10, 13125 Berlin (DE) on the dates indicated in the following table.

| Name of the | Accession | | |
|---|---|---|---|
| Cell Line | Number | Depositor | Date of Deposition |
| NM-F9 | DSM ACC 2606 | Nemod Biotherapeuticy | Aug. 14, 2003 |
| NM-H9D8 | DSM ACC 2806 | Glycotope GmbH | Sep. 15, 2006 |
| NM-H9D8-E6 | DSM ACC 2807 | Glycotope GmbH | Oct. 5, 2006 |
| NM-H9D8-E6Q12 | DSM ACC 2856 | Glycotope GmbH | Aug. 8, 2007 |

1. An antibody which is capable of specifically binding to glycosylated human LYPD3 at an epitope comprising an oligosaccharide structure selected from the group consisting of GalNAcα1-, sialylated GalNAcα1-, Galβ1-3GalNAcα1-, and sialylated Galβ1-3GalNAcα1-, which is attached to a serine or threonine residue of LYPD3.

2. The antibody according to claim 1, wherein the oligosaccharide structure is attached to a serine or threonine residue within positions 234 to 303 of SEQ ID NO: 137.

3. The antibody according to claim 1, wherein the epitope comprises one or more amino acids within positions 234 to 303 of SEQ ID NO: 137, and wherein in particular at least 50% of the amino acids of the epitope are present within positions 234 to 303 of SEQ ID NO: 137.

4. The antibody according to claim 1, which is capable of specifically binding to human LYPD3 glycosylated with any one or both of GalNAcα1- and Galβ1-3GalNAcα1-.

5. The antibody according to claim 1, which is capable of specifically binding to human LYPD3 glycosylated with Galβ1-3GalNAcα1-.

6. An antibody which is capable of binding to LYPD3 and which comprises
(i) a heavy chain variable region comprising the complementarity determining regions (CDRs) CDR-H1 having the amino acid sequence of SEQ ID NO: 1, CDR-H2 having the amino acid sequence of SEQ ID NO: 2 and CDR-H3 having the amino acid sequence of SEQ ID NO: 3, and
a light chain variable region comprising the complementarity-determining regions (CDRs) CDR-L1 having the amino acid sequence of SEQ ID NO: 4, CDR-L2 having the amino acid sequence of SEQ ID NO: 5 and CDR-L3 having the amino acid sequence of SEQ ID NO: 6; or
(ii) a heavy chain variable region comprising CDR-H1 having the amino acid sequence of SEQ ID NO: 9, CDR-H2 having the amino acid sequence of SEQ ID NO: 10 and CDR-H3 having the amino acid sequence of SEQ ID NO: 11, and
a light chain variable region comprising CDR-L1 having the amino acid sequence of SEQ ID NO: 12, CDR-L2 having the amino acid sequence of SEQ ID NO: 13 and CDR-L3 having the amino acid sequence of SEQ ID NO: 14; or
(iii) a heavy chain variable region comprising CDR-H1 having the amino acid sequence of SEQ ID NO: 17, CDR-H2 having the amino acid sequence of SEQ ID NO: 18 and CDR-H3 having the amino acid sequence of SEQ ID NO: 19, and
a light chain variable region comprising CDR-LI having the amino acid sequence of SEQ ID NO: 20, CDR-L2 having the amino acid sequence of SEQ ID NO: 21 and CDR-L3 having the amino acid sequence of SEQ ID NO: 22; or
(iv) a heavy chain variable region comprising CDR-H1 having the amino acid sequence of SEQ ID NO: 25, CDR-H2 having the amino acid sequence of SEQ ID NO: 26 and CDR-H3 having the amino acid sequence of SEQ ID NO: 27, and
a light chain variable region comprising CDR-L1 having the amino acid sequence of SEQ ID NO: 28, CDR-L2 having the amino acid sequence of SEQ ID NO: 29 and CDR-L3 having the amino acid sequence of SEQ ID NO: 30; or
(v) a heavy chain variable region comprising CDR-H1 having the amino acid sequence of SEQ ID NO: 33, CDR-H2 having the amino acid sequence of SEQ ID NO: 34 and CDR-H3 having the amino acid sequence of SEQ ID NO: 35, and
a light chain variable region comprising CDR-L1 having the amino acid sequence of SEQ ID NO: 36, CDR-L2 having the amino acid sequence of SEQ ID NO: 37 and CDR-L3 having the amino acid sequence of SEQ ID NO: 38; or
(vi) a heavy chain variable region comprising CDR-H1 having the amino acid sequence of SEQ ID NO: 41, CDR-H2 having the amino acid sequence of SEQ ID NO: 42 and CDR-H3 having the amino acid sequence of SEQ ID NO: 43, and
a light chain variable region comprising CDR-L1 having the amino acid sequence of SEQ ID NO: 44, CDR-L2 having the amino acid sequence of SEQ ID NO: 45 and CDR-L3 having the amino acid sequence of SEQ ID NO: 46; or
(vii) a heavy chain variable region comprising CDR-H1 having the amino acid sequence of SEQ ID NO: 49, CDR-H2 having the amino acid sequence of SEQ ID NO: 50 and CDR-H3 having the amino acid sequence of SEQ ID NO: 51, and
a light chain variable region comprising CDR-L1 having the amino acid sequence of SEQ ID NO: 52, CDR-L2 having the amino acid sequence of SEQ ID NO: 53 and CDR-L3 having the amino acid sequence of SEQ ID NO: 54; or
(viii) a heavy chain variable region comprising CDR-H1 having the amino acid sequence of SEQ ID NO: 57, CDR-H2 having the amino acid sequence of SEQ ID NO: 58 and CDR-H3 having the amino acid sequence of SEQ ID NO: 59, and
a light chain variable region comprising CDR-L1 having the amino acid sequence of SEQ ID NO: 60, CDR-L2 having the amino acid sequence of SEQ ID NO: 61 and CDR-L3 having the amino acid sequence of SEQ ID NO: 62; or
(ix) a heavy chain variable region and a light chain variable region according to any one of items (i) to (viii), above, comprising 1, 2 or 3 amino acid substitutions in total in the six CDR sequences.

7. The antibody according to claim 6, wherein
(i) the heavy chain variable region comprises the amino acid sequence of SEQ ID NO: 7, and the light chain variable region comprises the amino acid sequence of SEQ ID NO: 8; or
(ii) the heavy chain variable region comprises the amino acid sequence of SEQ ID NO: 15, and the light chain variable region comprises the amino acid sequence of SEQ ID NO: 16; or
(iii) the heavy chain variable region comprises the amino acid sequence of SEQ ID NO: 23, and the light chain variable region comprises the amino acid sequence of SEQ ID NO: 24; or
(iv) the heavy chain variable region comprises the amino acid sequence of SEQ ID NO: 31, and the light chain variable region comprises the amino acid sequence of SEQ ID NO: 32; or
(v) the heavy chain variable region comprises the amino acid sequence of SEQ ID NO: 39, and the light chain variable region comprises the amino acid sequence of SEQ ID NO: 40; or
(vi) the heavy chain variable region comprises the amino acid sequence of SEQ ID NO: 47, and the light chain variable region comprises the amino acid sequence of SEQ ID NO: 48; or
(vii) the heavy chain variable region comprises the amino acid sequence of SEQ ID NO: 55, and the light chain variable region comprises the amino acid sequence of SEQ ID NO: 56; or
(viii) the heavy chain variable region comprises the amino acid sequence of SEQ ID NO: 63, and the light chain variable region comprises the amino acid sequence of SEQ ID NO: 64; or
(ix) the heavy chain variable region and the light chain variable region comprise amino acid sequences which are at least 60% identical to the heavy chain variable region sequence and light chain variable region sequence, respectively, of any one of items (i) to (viii), above, over their entire length, wherein the antibody preferably is a humanized version of the antibody according to any one of items (i) to (viii), above.

8. The antibody according to claim 6, further comprising an Fc region.

9. (canceled)

10. The antibody according to claim 6, comprising
(i) a heavy chain variable region comprising the complementarity-determining regions (CDRs) CDR-H1 having the amino acid sequence of SEQ ID NO: 33, CDR-H2 having the amino acid sequence of SEQ ID NO: 34 and CDR-H3 having the amino acid sequence of SEQ ID NO: 35, and
a light chain variable region comprising the complementarity-determining regions (CDRs) CDR-L1 having the amino acid sequence of SEQ ID NO: 36, CDR-L2 having the amino acid sequence of SEQ ID NO: 37 and CDR-L3 having the amino acid sequence of SEQ ID NO: 38; or
(ii) a heavy chain variable region having an amino acid sequence which is at least 60% identical to the amino acid sequence of SEQ ID NO: 39 over its entire length and comprising CDR-H1 having the amino acid sequence of SEQ ID NO: 33, CDR-H2 having the amino acid sequence of SEQ ID NO: 34 and CDR-H3 having the amino acid sequence of SEQ ID NO: 35, and
a light chain variable region having an amino acid sequence which is at least 60% identical to the amino acid sequence of SEQ ID NO: 40 over its entire length and comprising CDR-LI having the amino acid sequence of SEQ ID NO: 36, CDR-L2 having the amino acid sequence of SEQ ID NO: 37 and CDR-L3 having the amino acid sequence of SEQ ID NO: 38;
wherein the antibody is capable of specifically binding to human LYPD3 glycosylated with any one or both of GalNAcα1- and Galβ1-3GalNAcα1-.

11. The antibody according to claim 6, comprising
(i) a heavy chain variable region comprising the complementarity-determining regions (CDRs) CDR-H1 having the amino acid sequence of SEQ ID NO: 9, CDR-H2 having the amino acid sequence of SEQ ID NO: 10 and CDR-H3 having the amino acid sequence of SEQ ID NO: 11, and
a light chain variable region comprising the complementarity-determining regions (CDRs) CDR-L1 having the amino acid sequence of SEQ ID NO: 12, CDR-L2 having the amino acid sequence of SEQ ID NO: 13 and CDR-L3 having the amino acid sequence of SEQ ID NO: 14; or
(ii) a heavy chain variable region having an amino acid sequence which is at least 60% identical to the amino acid sequence of SEQ ID NO: 15 over its entire length and comprising CDR-H1 having the amino acid sequence of SEQ ID NO: 9, CDR-H2 having the amino acid sequence of SEQ ID NO: 10 and CDR-H3 having the amino acid sequence of SEQ ID NO: 11, and
a light chain variable region having an amino acid sequence which is at least 60% identical to the amino acid sequence of SEQ ID NO: 16 over its entire length and comprising CDR-L1 having the amino acid sequence of SEQ ID NO: 12, CDR-L2 having the amino acid sequence of SEQ ID NO: 13 and CDR-L3 having the amino acid sequence of SEQ ID NO: 14;
wherein the antibody is capable of specifically binding to human LYPD3 glycosylated with Galβ1-3GalNAcα1-.

12. The antibody according to claim 6, being a humanized antibody wherein
(i) the heavy chain variable region comprises the amino acid sequence of SEQ ID NO: 139, and the light chain variable region comprises the amino acid sequence of SEQ ID NO: 144; or
(ii) the heavy chain variable region comprises the amino acid sequence of SEQ ID NO: 140, and the light chain variable region comprises the amino acid sequence of SEQ ID NO: 144; or
(iii) the heavy chain variable region comprises the amino acid sequence of SEQ ID NO: 141, and the light chain variable region comprises the amino acid sequence of SEQ ID NO: 144; or
(iv) the heavy chain variable region comprises the amino acid sequence of SEQ ID NO: 142, and the light chain variable region comprises the amino acid sequence of SEQ ID NO: 144; or
(v) the heavy chain variable region comprises the amino acid sequence of SEQ ID NO: 142, and the light chain variable region comprises the amino acid sequence of SEQ ID NO: 143; or
(vi) the heavy chain variable region comprises the amino acid sequence of SEQ ID NO: 142, and the light chain variable region comprises the amino acid sequence of SEQ ID NO: 145; or
(vii) the heavy chain variable region comprises the amino acid sequence of SEQ ID NO: 142, and the light chain variable region comprises the amino acid sequence of SEQ ID NO: 146; or
(viii) the heavy chain variable region comprises the amino acid sequence of SEQ ID NO: 138, and the light chain variable region comprises the amino acid sequence of SEQ ID NO: 145; or
(ix) the heavy chain variable region comprises the amino acid sequence of SEQ ID NO: 140, and the light chain variable region comprises the amino acid sequence of SEQ ID NO: 145.

13. The antibody according to claim 1, being an IgG1-type, IgG2-type, IgG3-type or IgG4-type antibody, in particular an IgG1-type antibody.

14. A conjugate comprising the antibody according to claim 1 conjugated to a further agent.

15. The conjugate according to claim 14, wherein the further agent is a cytotoxic agent, tumor-specific antibody or immune checkpoint blocking or activating antibody.

16. The conjugate according to claim 14, being a chimeric antigen receptor.

17. A nucleic acid encoding the antibody according to claim 1.

18. An expression cassette or vector comprising the nucleic acid according to claim 17 and a promoter operatively connected with said nucleic acid.

19. A host cell comprising the nucleic acid according to claim 17.

20. The host cell according to claim 19, wherein the host cell is white blood cell.

21. A composition comprising the antibody of claim 1.

22. The composition according to claim 21 which further comprises one or more components selected from the group consisting of solvents, diluents and excipients.

23. (canceled)

24. A method of treating cancer, adolescent idiopathic scoliosis, cholesteryl ester transfer protein (CETP) deficiency, fish-eye disease, combined hyperlipidemia, citrullinemia or familial hypercholesterolemia in a patient comprising administering to the patient the antibody of claim 1.

25. The antibody according to claim 24, wherein the cancer is selected from the group consisting of head and neck cancer, colon cancer, colorectal cancer, hepatocellular carcinoma, skin cancer, cervical cancer, breast cancer, ovarian cancer, prostate cancer, renal cancer, esophageal cancer, lung cancer, and genital region cancer, in particular non-small cell lung cancer (NSCLC) and squamous cell carcinoma (SCC).

26. A nucleic acid encoding the conjugate according to claim 14, wherein the further agent is a polypeptide or protein fused to the antibody.
